(19) United States
(12) Patent Application Publication
(10) Pub. No.: US 2026/0240890 A1
(43) Pub. Date: Aug. 20, 2026

(54) SHORT INTERSPERSED NUCLEAR REPEAT ELEMENT RNA OF IMPRINTED MICRO-RNA CLUSTERS MEDIATE INTRINSIC TYPE III INTERFERON-DRIVEN VIRAL RESISTANCE

(71) Applicant: UNIVERSITY OF SOUTH FLORIDA, Tampa, FL (US)

(72) Inventor: Hana TOTARY-JAIN, Tampa, FL (US)

(21) Appl. No.: 19/161,289

(22) PCT Filed: Mar. 4, 2024
(86) PCT No.: PCT/US2024/018441

§ 371 Date: Sep. 1, 2025

Related U.S. Application Data
(60) Provisional application No. 63/488,119, filed on Mar. 2, 2023.
(60) Provisional application No. 63/490,751, filed on Mar. 16, 2023.
(60) Provisional application No. 63/490,753, filed on Mar. 16, 2023.

(51) Int. Cl. A61K 31/7115; A61K 9/00; A61K 31/713; A61P 31/12; C12Q 1/68

(52) U.S. Cl. CPC A61K 31/7115; A61K 9/0034; A61K 31/713; A61P 31/12; C12Q 1/68; C12Q 2600/106

(57) ABSTRACT

The present disclosure describes methods of inducing type III IFN stimulated genes in fetal trophoblast cells to decrease or inhibit vertical transmission of viral infection to the fetus. The method comprises induction of short interspersed nuclear elements in a microRNA cluster.

### CROSS-REFERENCE TO RELATED APPLICATIONS

This application claims priority to U.S. Provisional Application No. 63/488,119 filed on Mar. 2, 2023, U.S. Provisional Application No. 63/490,751 filed on Mar. 16, 2023, and U.S. Provisional Application No. 63/490,753 filed on Mar. 16, 2023, the content of which are incorporated by reference in their entireties.

### STATEMENT REGARDING FEDERALLY SPONSORED RESEARCH

This invention was made with government support under grant R01HL128411 awarded by the National Institutes of Health. The government has certain rights in this invention.

### REFERENCE TO AN ELECTRONIC SEQUENCE LISTING

The contents of the electronic sequence listing (173738.02732.xml; Size: 81,449 bytes; and Date of Creation: Mar. 4, 2024) is herein incorporated by reference in its entirety.

### BACKGROUND

The placenta in eutherian mammals occupies a unique immunological niche, balancing tolerance of the semi-allogenic fetus with protection of the fetus from invading pathogens (Ander et al., 2019). Despite the structural and functional differences between the human and mouse placentas, both are highly invasive, ‘hemochorial’ in which the fetal trophoblast is bathed in maternal blood over a long gestational period (Soares et al., 2018). These factors increase the risk of vertical transmission of pathogens to the fetus. Thus, placental trophoblast in both species have evolved unique physical and immunological antiviral barriers at the maternal-fetal interface (Megli and Coyne, 2022). Unlike somatic cells that require recognition of pathogen-associated molecular patterns (PAMPs) by pattern recognition receptors (PRRs) for interferon (IFN) induction, placental trophoblast constitutively releases type III interferons (IFNL), which act in both autocrine and paracrine fashions to confer viral resistance (Bayer et al., 2016; Corry et al., 2017). In fact, pregnant mice lacking IFNL signaling are more permissive to Zika virus (ZIKV) vertical transmission (Jagger et al., 2017). Unlike type I IFNs, which are induced by viral infections and often cause pregnancy complications including spontaneous abortions and growth restriction (Yockey et al., 2018), constitutive production of type III IFNs protects the fetus from infection without causing deleterious obstetrical consequences. However, the mechanisms that induce the intrinsic IFNL expression in human and mouse placentas are unknown.

### SUMMARY

One aspect of the present invention provides a method for reducing the likelihood of amniotic fluid infection and/or fetal infection, or treating amniotic fluid infection and/or fetal infection, the method comprising: vaginally administering to a pregnant female a composition comprising an in vitro transcribed (IVT) RNA comprising short interspersed nuclear elements (SINEs), or fragments thereof. In some embodiments, the administration is topical, including as a cream, lotion, gel, suppository or insert. In some embodiments the infection is viral, bacterial or fungal.

Another aspect of the present invention provides a method for preparing a cell, tissue, or an organ for transplant, the method comprising: contacting the cell, tissue, or organ with a composition comprising an in vitro transcribed (IVT) RNA comprising short interspersed nuclear elements (SINEs), or fragments thereof. In some embodiments, the transplant recipient is administered IVT RNA comprising SINEs, or fragments thereof, prior to transplantation.

Another aspect of the present invention provides a method of enhancing immunotolerance in subject in need thereof, the methods comprising: administering a composition comprising an in vitro transcribed (IVT) RNA comprising short interspersed nuclear elements (SINEs), or fragments thereof, optionally, wherein the subject is scheduled for cell, organ, or tissue transplant, and/or wherein the subject is immunocompromised, e.g., comprises an immunodeficiency disorder.

Another aspect of the present invention provides a method of treating wounded tissue in a subject in need thereof, the method comprising: administering to the wounded tissue a composition comprising an in vitro transcribed (IVT) RNA comprising short interspersed nuclear elements (SINEs), or fragments thereof. In some embodiments, the wound is a skin wound and the composition is administered topically. In some embodiments the wound is in lung tissue, and the composition is administered via inhalation. In some embodiments, the wound is an eye wound, and the composition is administered topically to the wounded eye.

The IVT RNA of the present invention may comprise at least one modified nucleotide. The modified nucleotide may comprise one of pseudouridine, NI-methylpseudouridine, 5-methylcytosine (m5C), 5-methyluridine (m5U), 2′-O-methyluridine (Um or m2′OU), 2-thiouridine (s2U), or N6-methyladenosine (m6A)). In some embodiments, the SINE comprises Alu repeats.

In some embodiments, the methods provided herein comprises, prior to administration, determining the level of Alu RNA in a biological sample from the subject. In some embodiments, detecting the level of Alu RNA comprises measuring the level of a small cytoplasmic Alu (sc-Alu) RNA in the sample. In some embodiments, the tissue sample is from a tissue or organ biopsy, placenta, blood, serum, plasma, vaginal discharge, urine, lymphatic fluids or amniotic fluid.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Non-limiting embodiments of the present disclosure will be described by way of example with reference to the accompanying Figures, which are schematic and are not intended to be drawn to scale. In the Figures, each identical or nearly identical component illustrated is typically represented by a single numeral. For purposes of clarity, not every component is labeled in every Figure, nor is every component of each embodiment of the invention shown where illustration is not necessary to allow those of ordinary skill in the art to understand the invention.

FIGS. 1A-1M. Transcriptional activation of C19MC induces a strong type III IFN response. (FIGS. 1A-1D) AD-293 cells transfected with 759-SAM (759), 620-SAM (620), or GFP for 72 h. Differential expression analysis of major-strand mature miRNAs (>±10-fold, adjusted p<0.05) with C19MC miRNA marked in red and other miRNA in blue (FIGS. 1A and 1B), hallmark GSEA plot of IFNa response (FIG. 1C), and heatmap representing gene ontology biological process of defense response to virus (FIG. 1D) are shown. (FIGS. 1E-1J) AD-293 cells transfected with BB-SAM (BB), 759, or GFP for 72 h. RT-qPCR for representative C19MC miRNAs normalized to U18 (FIG. 1E); representative agarose gels of IFNL2/3 and GAPDH RT-PCR (FIG. 1F); RT-qPCR for IFNL2/3, IFNA2, and IFNB1 normalized to GAPDH (FIG. 1G); ELISA-based quantification of IFNL1/3 in the supernatant (FIG. 1H); representative immunoblot and densitometric quantification of IRF7 normalized to GAPDH (FIG. 1I); and RT-qPCR of representative ISGs normalized to GAPDH (FIG. 1J) (one-way ANOVA with Dunnett's multiple comparison test) are shown. (FIGS. 1K-1M) HTR8/SVneo cells transfected with 759 or BB for 72 h. Representative RT-qPCR for miR-517a normalized to U18 (FIG. 1K), agarose gels of IFNL2/3 and GAPDH RT-PCR (FIG. 1L), and RT-qPCR of representative ISGs normalized to GAPDH (FIG. 1M) (unpaired two-tailed t test with Welch's correction) are shown. Data represent the mean±SEM of a representative experiment performed in triplicate performed in at least three independent experiments (FIGS. 1A-J). *p<0.05, **p<0.01, ***p<0.001 and ****p<0.0001, versus BB. ns, not significant and ND, not detected.

FIGS. 2A-2I. Activation of C19MC preferentially induces IFNL in a miRNA-independent fashion. 293T and DICER-KO (KO) cells transfected with 759-SAM (759) or BB-SAM (BB) for 72 h. Differential expression analysis of mature miRNAs (>±10-fold, adjusted p<0.05) with C19MC miRNA marked in red and other miRNA in blue (FIGS. 2A and 2B); RT-qPCR for miR-517a normalized to U18 (FIG. 2C); hallmark GSEA plot of IFNa response (FIG. 2D); heatmap representing gene ontology biological process of negative regulation of viral process (FIG. 2E); RT-qPCR for IFNL2/3, IFNA2, and IFNB1 normalized to GAPDH (FIG. 2F); representative agarose gels of IFNL2/3 and GAPDH RT-PCR (FIG. 2G); ELISA-based quantification of IFNL1/3 and IFNA (FIG. 2H); and RT-qPCR of representative ISGs normalized to GAPDH (FIG. 2I) (unpaired two-tailed t test with Welch's correction) are shown. Data represent the mean±SEM of at least three independent experiments, each performed in triplicate (FIGS. 2C, 2F, 2G, and 2I), or at least two independent experiments performed in triplicate (FIG. 2H). *p<0.05 and **p<0.01 versus BB. ns, not significant and ND, not detected. See also FIG. 9 and Tables 1 and 3.

FIGS. 3A-3H. C19MC activation increases Alu dsRNA and protects against viral infection in a miRNA-independent fashion. (FIGS. 3A and 3B) RT-qPCR for the indicated viral RNA normalized to GAPDH in 293T (FIG. 3A) and DICER-KO cells (FIG. 3B) transfected with 759-SAM (759) or BB-SAM (BB) for 60-72 h and infected with either VSV for 8 h or with ZIKV or RSV for 24 h (unpaired two-tailed t test). (FIGS. 3C and 3D) RT-qPCR for CYP19A1 and IFNL2/3 normalized to GAPDH and miR-519 normalized to U18 in 293T (FIG. 3C) and DICER-KO (FIG. 3D) cells transfected with 125.3-SAM (125.3), 759, or BB for 72 h (one-way ANOVA with Dunnett's multiple comparison test). (FIGS. 3E and 3F) Representative agarose gels of Alu and GAPDH RT-PCR with control Alu PCR products using total RNA (bottom panel) (FIG. 3E) and densitometric quantification of fl-Alu to sc-Alu ratio normalized to GAPDH (FIG. 3F) of 293T and DICER1-KO cells transfected with 759 or BB for 72 h and control HeLa cells after heat shock recovery (unpaired two-tailed t test). (FIGS. 3G and 3H) RT-qPCR of indicated genes normalized to GAPDH of DROSHA-KO 293T cells transfected with 759 or BB for 72 h (unpaired two-tailed t test with Welch's correction). Data represent the mean±SEM of at least three independent experiments, each performed in triplicate (FIGS. 3E and 3F), two independent experiments performed in triplicate (FIGS. 3C and 3G), or a representative experiment performed in triplicate. (FIGS. 3A, 3B, 3D, and 3H). *p<0.05, **p<0.01, ***p<0.001 and ****p<0.0001 vs. BB-transfected cells. See also FIG. 10 and Tables 4 and 5.

FIGS. 4A-4J. C19MC Alu RNA induces IFN through RLR and PKR signaling pathways. (FIG. 4A) Representative immunoblot for MAVS and GAPDH in 293T and MAVS-KO cells. (FIG. 4B) RT-qPCR for miR-517a normalized to U18 and IFNL2/3 and IFNA2 normalized to GAPDH in 293T and MAVS-KO 293T cells transfected with 759-SAM (759) or BB-SAM (BB) for 72 h. (FIGS. 4C and 4D) Representative immunoblot for MAVS and GAPDH (FIG. 4C) and RT-qPCR for MAVS, IFNL2/3, and IFNA2 normalized to GAPDH (FIG. 4D) in DICER-KO cells transfected with MAVS-specific siRNA or control siRNA for 24 h, followed by 759 or BB transfection for 48 h. (FIGS. 4E and 4F) RT-qPCR for miR-517a normalized to U18 (E) and TLR3, IFNL2/3, and IFNA2 normalized to GAPDH (FIGS. 4E and 4F) in 293T (FIG. 4E) and DICER-KO (FIG. 4F) cells transfected with TLR3-specific siRNA or control siRNA for 24 h, followed by 759 or BB transfection for 48 h. (FIGS. 4G-4J) Representative immunoblot for PKR and GAPDH (FIGS. 4G and 4I); RT-qPCR for miR-517a normalized to U18 (FIG. 4H); and PKR, IFNL2/3, and IFNA2 normalized to GAPDH (FIGS. 4H and 4J) in 293T (FIGS. 4G and 4H) and DICER-KO (FIGS. 4I and 4J) cells transfected with PKR-specific siRNA or control siRNA for 24 h, followed by transfection with 759 or BB for 48 h. Data represent the mean±SEM of a representative experiment of three independent experiments performed in triplicate. One-way ANOVA with Tukey's multiple comparison test. *p<0.05 and ***p<0.001 vs. 759-transfected 293T cells (FIG. 4B). *p<0.05, **p<0.01 and ****p<0.0001 vs. control siRNA and 759-transfected cells (FIGS. 4D-F4, 4H, and 4J). ns, not significant.

FIGS. 5A-5I. SINE RNA co-localizes with C19MC and C2MC miRNAs in human and mouse placenta. (FIG. 5A) Representative in situ hybridization images of miR-517a/b (purple), Alu (purple), or control scramble probes in term human placentas (n=3) pre-treated with DNase I, RNase A, or vehicle control. Nuclei were counterstained with nuclear fast red. Scale bars: 100 mm; original magnification, 340. (FIG. 5B) Representative in situ hybridization images of miR-517a/b, Alu, or control scramble probes and immunostaining for cytokeratin-7 (brown) and vimentin (pink) in 1st trimester human placentas. Scale bars: 200 mm; original magnification, 310 and insets 320 and 340. (FIG. 5C) Representative immunofluorescent staining of human term placental sections with dsRNA-specific J2, mitochondria marker HSP60, or control secondary antibody. Nuclei were counterstained with DAPI. Scale bars: 50 mm; original magnification, 1203. (FIG. 5D) Representative agarose gels of Alu and GAPDH RT-PCR and control Alu PCR products using total RNA (bottom panel) in term human placenta (PL), human adult cardiac left ventricle (LV) (n=4 each), and control HeLa cells after heat shock recovery. (FIG. 5E) Representative in situ hybridization images of miR-669a-3p (purple), B1 (purple), or control scramble probes in WT E18.5 mouse placentas. Nuclei were counterstained with nuclear fast red. Scale bars: 500 mm; original magnification, 33 and 320 (insets). (FIG. 5F) Representative agarose gels of B1 and Polra2 RT-PCR product of E11.5 mouse placentas (top) and mTS cells derived from WT and C2MC^{Δ/Δ} (bottom). (FIG. 5G) RT-qPCR of miR-467a normalized to snoRNA202 of E11.5 mouse placentas and mTS cells derived from WT and C2MC^{Δ/Δ} mice (unpaired two-tailed t test with Welch's correction). (FIG. 5H) Representative dot blot and densitometric quantification of dsRNA detected by J2 antibody in total RNA extracted from WT and C2MC^{Δ/Δ}-derived mTS cells (unpaired two-tailed t test). (FIG. 5I) SARS-COV-2 induces Alu RNA in human lungs and hCA. Representative images of in-situ hybridization for Alu probe (purple) or scrambled control of lung biopsy and hCA from COVID-19 positive and negative patients. Nuclei were counterstained with nuclear fast red. Original magnification 20×. Data represent the mean±SEM of a representative experiment of three placentas and three independent experiments performed in triplicate using a single clone of WT or C2MC^{Δ/Δ} mTS cells (FIG. 5G) and in triplicate using a single clone of WT or C2MC^{Δ/Δ} mTS cells (FIG. 5H). *p<0.05 and **p<0.01 vs. WT. STB, syncytiotrophoblast; CTB, cytotrophoblast; EVT, extravillous trophoblasts; st, spongiotrophoblast; la, labyrinth; ma, maternal decidua. See also Table 5.

FIGS. 6A-6H. C2MC B1 RNA induces IFN and antiviral protection in mTS cells in a miRNA-independent manner and restricts placental vertical transmission in vivo. (FIG. 6A) RT-qPCR for Ifnl3, Ifna2, and Ifnb1 normalized to Polr2a in WT and C2MC^{Δ/Δ} mTS cells (unpaired two-tailed t test). (FIGS. 6B and 6C) RT-qPCR for ZIKV normalized to Polr2a in E14.5 WT (n=17) and C2MC^{Δ/Δ} (n=14) placentas (FIG. 6B) and fetal heads (FIG. 6C) obtained from pregnant dams infected with ZIKV on E9.5 after IP injection of anti-mouse IFNAR1 mAb a day prior (Mann-Whitney non-parametric test). (FIG. 6D) RT-qPCR for Ifnl3, Ifna2, and Ifnb1 and ZIKV normalized to Polr2a in WT and C2MC^{Δ/Δ}mTS cells infected with ZIKV for 24 h (unpaired two-tailed t test). (FIG. 6E) RT-qPCR of indicated C2MC miRNA normalized to snoRNA202 and Ifnl3 normalized to Polr2a in C2MC^{Δ/Δ}mTS cells transfected with miR-467b-, miR-466b/c/p-, or control miR-mimic compared with WT mTS cells transfected with control miR-mimic for 24 h (one-way ANOVA with Dunnett's multiple comparison test). (FIG. 6F) RT-qPCR for Ifnl3 and for ZIKV normalized to Polr2a in C2MC^{Δ/Δ}mTS cells transfected as in (FIG. 6E) for 4 h and then infected with ZIKV for 24 h (one-way ANOVA with Dunnett's multiple comparison test). (FIG. 6G) RT-qPCR for Ifnl3, Ifna2, and Ifnb1 normalized to Polr2a in C2MC^{Δ/Δ} mTS cells transfected for 24 h with either IVT GFP mRNA (control) or B1 SINE RNA in the forward direction (B1) (unpaired two-tailed t test). (FIG. 6H) RT-qPCR for Ifnl3, Ifna2, and Ifnb1 and ZIKV normalized to Polr2a in C2MC^{Δ/Δ} mTS cells transfected as in (FIG. 6G) for 4 h followed by ZIKV infection for 24 h (unpaired two-tailed t test with Welch's correction). Data represent the mean±SEM of a representative experiment performed in triplicate of at least three in-dependent experiments. *p<0.05, **p<0.01, ***p<0.001 and ****p<0.0001 vs. C2MC^{Δ/Δ} mTS cells (FIGS. 6A and 6D), vs. WT (FIGS. 6B and 6C), vs. C2MC^{Δ/Δ} mTS cells transfected with control miR-mimic (FIGS. 6E and 6F) or control GFP mRNA (FIGS. 6G and 6H). See also FIG. 11.

FIGS. 7A-7C. Genomic arrangement of C19MC and C2MC. (FIG. 7A) C19MC genomic features as viewed on chromosome 19 of the human genome (GRCh38/hg38). Note the upstream CpG island (green rectangle), the 46 miRNA precursors, the SINEs, and the multiple alignments showing its human and non-human primate-specific locus. (FIG. 7B) C2MC genomic features as viewed on chromosome 2 of the mouse genome (GRCm38/mm 10). Note the 72 miRNA precursors on intron 10 of Sfmbt2, the SINEs, and the multiple alignments showing its rodent-specific locus. (FIG. 7C) RT-qPCR for the indicated C19MC miRNAs normalized to U18 in AD-293 cells transfected with 759-SAM (759), 620-SAM (620) or GFP for 72 hours (a representative experiment performed in triplicate) and in human term placental tissue (n=3).

FIGS. 8A-8D. C19MC activation induces type III IFN response in a autocrine/paracrine manner. (FIG. 8A) Hallmark gene set enrichment bar plot of AD-293 cells transfected with 759 or 620 for 72 hours compared to GFP transfected cells. (FIG. 8B) Representative agarose gels of IFNA2, IFNB1 and GAPDH RT-PCR, in AD-293 cells transfected with 759, BB-SAM (BB), GFP or control poly I:C transfected cells. (FIG. 8C) RT-qPCR for ISG15 and OAS1 normalized to GAPDH in AD-293 cells transfected with 759, BB or GFP and treated with anti-INFL3 or vehicle control for 72 hours (One-way ANOVA with Tukey's multiple comparison test). (FIG. 8D) Representative agarose gels of IFNA2, IFNB1 and GAPDH RT-PCR, in HTR8/SVneo cells transfected with 759, BB or control poly I:C transfected AD-293 cells. Data represent fold change of a representative experiment performed in triplicate of two independent experiments (FIG. 8C). **p<0.01 and ****p<0.0001 versus 759 transfected vehicle control treated AD-293 cells (FIG. 8C).

FIGS. 9A-9D. Transcriptional activation of C19MC induces IFN and ISGs independent of miRNAs. (FIG. 9A) Representative immunoblot for DICER1 and anti-alpha tubulin 1B (TUBA1B) in 293T and DICER-Ko cells. (FIGS. 9B-9D) 293T and DICER-Ko cells transfected with 759-SAM (759) or BB-SAM (BB) for 72 hours. Hallmark gene set enrichment bar plots of 293T (FIG. 9B) and DICER-Ko cells (FIG. 9C) and a representative agarose gel of IFNA2, IFNB1 and GAPDH RT-PCR with control poly I:C transfected AD-293 cells (FIG. 9D).

FIGS. 10A-10F. C19MC transcriptional activation increases dsRNA and protects against SARS-COV-2, related to FIG. 3 and Table 4. (FIG. 10A) RT-qPCR for SARS-COV-2 viral RNA normalized to GAPDH in 293T-ACE2 cells transfected with 759-SAM (759) or BB-SAM (BB) for 60 hours and infected with SARS-COV-2 for 24 hours (Unpaired two-tailed t test). (FIG. 10B) CYP19A1 genomic features spanning 130 kb as viewed on chromosome 15 of the human genome (GRCh38/hg38). Note the low density of SINEs. (FIGS. 10C, 10D) Representative dot blots of in vitro transcribed Alu RNA in the reverse (R), forward (F) directions, combination R+F and negative control dsDNA (DNA) PCR product that was used as template for Alu in vitro transcription (FIG. 10C) and total RNA isolated 72 hours after transfection of 293T and DICER-Ko cells with 759 or BB (FIG. 10D) using dsRNA J2 monoclonal antibody. (FIG. 10E) Representative immunoblot of DROSHA and GAPDH in 293T and DROSHA-knockout (DROSHA-ko) 293T cells. (FIG. 10F) Representative agarose gels of IFNL2/3, IFNA2, IFNB1 and GAPDH RT-PCR, in DROSHA-Ko cells transfected with 759, BB for 72 hours or control poly I:C transfected AD-293 cells. Data represent the mean±SEM of a representative experiment performed in triplicate of three independent experiments (FIGS. 10A, 10F). *p<0.05 versus BB.

FIGS. 11A-11D. C2MC^{Δ/Δ} abolishes the constitutive IFN expression in mouse placenta and mediates protection against ZIKV, related to FIG. 6. (FIG. 11A) GEO2R microarray expression profile analysis of differentially expressed IFNs (Adjusted p<0.05, fold change>±2) in E11.5 placentas of wild-type (WT) and C2MC^{Δ/Δ}C2MC−/−) mice from GEO: GSE82055 [S1]. (FIG. 11B) RT-qPCR of ZIKV normalized to Polr2a in maternal spleens of E14.5 pregnant dams infected with ZIKV on E9.5, after IP injection of anti-mouse IFNAR1 mAb a day prior. Data represent the mean±SEM (P=0.5667; Kruskal-Wallis statistic=1.952). (FIG. 11C) RT-qPCR of Ifna2 and Ifnb1 normalized to Polr2a in C2MCΔ/Δ mTS cells transfected with miR-467b-, miR-466b/c/p- or control miR-mimic compared to WT mTS cells transfected with control miR-mimic for 24 hours (One-way ANOVA with Dunnett's multiple comparison test). (FIG. 11D) RT-qPCR for Ifna2 and Ifnb1 and for ZIKV normalized to Polr2a in C2MCΔ/Δ mTS cells transfected as in (FIG. 11C) for 4 hours and then infected with ZIKV for 24 hours (One-way ANOVA with Dunnett's multiple comparison test). Data represent the mean±SEM of a representative experiment performed in triplicate. ns, not significant vs WT (FIG. 11B) and vs C2MCΔ/Δ mTS cells transfected with control miR-mimic (FIGS. 11C, 11D).

FIGS. 12A-12D. Hallmark GSEA plot of allograft rejection (A and C), and heatmap representing differentially expressed allograft rejection upregulated and down regulated genes (B and D) of DICER-KO cells transfected with 759-SAM (759) or BB-SAM (BB) for 72 h (A and B), and E11.5 placenta derived from C2MC^{Δ/Δ} (KO) or C2MC^{+/+} (WT) mice (C and D).

FIGS. 13A-13D. (A). GSEA bar plot of DICER-KO cells transfected with 759-SAM vs. BB-SAM. (B-D) Heatmap representing complement (B), cytokine pathways (C), and AMPs (D) in E11.5 placenta derived from C2MC^{Δ/Δ} (KO) vs C2MC^{+/+} (WT) mice.

### DETAILED DESCRIPTION

Disclosed are agents, compositions, and methods reducing the likelihood of infection or immune response using an in vitro transcribed (IVT) RNA comprising short interspersed nuclear elements (SINEs), or fragments thereof. The methods comprise induction of SINEs in a microRNA cluster. The agents or methods may be used to induce type III IFN stimulated genes to decrease or inhibit transmission of viral infection, including to a fetus. One aspect of the disclosure includes methods for reducing the likelihood of amniotic fluid infection and/or fetal infection, or treating amniotic fluid infection and/or fetal infection, using the in vitro transcribed (IVT) RNA having short interspersed nuclear elements (SINEs), or fragments thereof. Other aspects include methods for preventing, inhibiting, or mediating an immune response, such as with immunosuppression condition, with an in vitro transcribed (IVT) RNA agent or composition. Such methods include enhancing immunotolerance.

The placenta is a unique immunological niche that tolerates the semi-allogenic fetus while protecting the immunologically-vulnerable fetus against pathogens. Primates and rodents have evolved invasive ‘hemochorial’ placentas where the fetal trophoblast is bathed in maternal blood, thus increasing the risk of pathogen vertical transmission. Unlike somatic cells that require pathogen-associated molecular patterns to stimulate interferon (IFN) production, the placental trophoblast constitutively produces type III interferons (IFNL), even in the absence of viral infections, through an unknown mechanism. The primate-specific miRNA cluster on chromosome 19 (C19MC) and in the rodent-specific microRNA cluster on chromosome 2 (C2MC) are among the largest miRNA clusters in humans and mice, respectively, and are constitutively expressed in the placenta exclusively from the paternal allele. While many studies have focused on the iRNA of these clusters, the present disclosure describes the important role of SINEs in this cluster. The present disclosure provides novel methods of detecting SINE RNA, and use of these methods to decrease fetal viral infection and/or decreasing viral vertical transmission.

### Methods

C19MC is the largest human miRNA gene cluster, extending over a ~100 kb long region on chromosome 19. It consists of 46 genes which encode 59 mature miRNAs. The C19MC miRNA cluster is only found in primate (including human) genomes and expresses miRNAs in the placenta, testis, embryonic stem cells, and some tumors. They are also expressed highly in trophoblast-derived vesicles, including exosomes. Expression of the C19MC miRNA cluster is repressed in other tissues by DNA and/or histone methylation. C19MC miRNAs have been shown to be among the most expressed miRNAs in the human placenta and are also found in the serum of pregnant women.

Abnormal regulation of C19MC miRNA genes causes a variety of human diseases, including preeclampsia, and cancers such as hepatocellular carcinoma, breast cancer, parathyroid tumor, brain cancer, lung cancer, bladder cancer, infantile hemangioma, and infant brain cancers. C19MC also plays an important role in embryonic development and cellular differentiation.

The C19MC miRNA cluster is flanked by Alu SINE repeats. An Alu element is a short stretch of DNA originally characterized by the action of the Arthrobacter luteus (Alu) restriction endonuclease. Alu elements are the most abundant transposable elements, containing over one million copies dispersed throughout the human genome. They belong to a class of retroelements termed SINEs (short interspersed nuclear elements) and are primate specific. Alu elements are about 300 base pairs long with a typical structure of 5′-Part A-ASTACA6-Part B-PolyA Tail-3′, where Part A and Part B (also known as “left arm” and “right arm”) are similar nucleotide sequences. Many individual Alu elements have wide-ranging influences on gene expression, including influences on polyadenylation, alternative splicing, ADAR (adenosine deaminase that acts on RNA) editing, and translation regulation.

Similar to humans, mice have a rodent-specific miRNA cluster, known as C2MC, which is maternally imprinted in the placenta. C2MC is composed of 72 miRNA precursor sequences flanked by an Alu-like family of SINES found in rodents known as BI elements. While the present invention is directed towards the detection of Alu RNA, the methods may be used, with species specific primers and probes, as demonstrated by the use of the same methods to detect BI elements in the mouse, as demonstrated in Figures included herein.

One aspect of the present invention provides a method for reducing the likelihood of amniotic fluid infection and/or fetal infection, or treating amniotic fluid infection and/or fetal infection. In some embodiments, the method comprises vaginally administering to a pregnant female a composition comprising an in vitro transcribed (IVT) RNA comprising short interspersed nuclear elements (SINEs), or fragments thereof.

As used herein, the term “administering” an agent, such as an IVT RNA described herein to an animal or cell, is intended to refer to dispensing, delivering or applying the substance to the intended target. In terms of the therapeutic agent composition, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

In the disclosed methods, the IVT RNA may be administered by any suitable route of administration. Suitable routes of administration may include oral administration, topical administration, subdermal administration, and/or subcutaneous administration. In some embodiments, the RNA is administered locally at the site of infection. In some embodiments, the RNA is administered ex vivo to cells, a substrate, or tissue prior to administration to the patient. Examples of topical administration include, but are not limited to gels, ointments, creams, suppository, insert and suspensions, dressings, bioerodible patches, ex-vivo cells, or transdermal delivery with chemical or physical approaches. In the disclosed methods, the RNA may be administered alone or as part of a combination therapy or complimentary therapy.

This disclosure also describes use of a composition or formulation that includes an Alu RNA, such as in vitro transcribed Alu RNA, as described, and a delivery vehicle. The composition or formulation can have anti-viral, antimicrobial, and/or antifungal activity and may, therefore, be an anti-viral, antimicrobial, and/or antifungal agent. In the embodiments, the delivery vehicle may be an in vivo delivery vehicle.

The described Alu RNA, for example in vitro transcribed Alu RNA, may be formulated in a composition or formulation along with a “carrier.” As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. In embodiments, the composition or formulation may a cream, lotion, emollient, spray, or aerosol.

The carrier may be a pharmaceutically acceptable carrier. By “pharmaceutically acceptable” is meant a material that is not biologically or otherwise undesirable, i.e., the material may be administered to an individual along with Alu RNA, such as in vitro transcribed Alu RNA, without causing any undesirable biological effects or interacting in a deleterious manner with any of the other components of the pharmaceutical composition in which it is contained.

The Alu RNA, such as in vitro transcribed Alu RNA, may be formulated into a pharmaceutical composition. The pharmaceutical composition may be formulated in a variety of forms adapted to a preferred route of administration. Thus, a composition can be delivered or administered via known routes including, for example, oral, parenteral (e.g., intradermal, transcutaneous, subcutaneous, etc.), or topical (e.g., intranasal, intrapulmonary, intramammary, intravaginal, intrauterine, intradermal, transcutaneous, rectally, etc.). A composition or formulation also can be administered via a sustained or delayed release.

In some embodiments, the RNA can be combined with a carrier or excipient. RNA may be administered as a pharmaceutical composition comprising one or more RNAs in combination with one or more pharmaceutically acceptable carriers or excipients. Such compositions may be aqueous solutions, emulsions, creams, ointments, suspensions, gels, liposomal suspensions, and the like. Suitable carriers (excipients) include water, saline, Ringer's solution, dextrose solution, and solutions of ethanol, glucose, sucrose, dextran, mannose, mannitol, sorbitol, polyethylene glycol (PEG), phosphate, acetate, gelatin, collagen, CARBOPOL®, vegetable oils, and the like. One may additionally include suitable preservatives, stabilizers, antioxidants, antimicrobials, and buffering agents, for example, BHA, BHT, citric acid, ascorbic acid, tetracycline, and the like. Cream or ointment bases useful in formulation include lanolin, SILVADENE®, AQUAPHOR®, and the like. Other topical formulations include aerosols, conditioners, bandages and other wound dressings. Alternatively, one may incorporate or encapsulate the RNA in a suitable polymer matrix or membrane, thus providing a sustained-release delivery device suitable for implantation near the site to be treated locally.

In the disclosed methods, the infection may be a viral infection, bacterial infection or fungal infection. Reducing the likelihood of amniotic fluid infection may include increasing resistance to fetal infection or decreasing the incidence of infection of the fetus or decreases in fetal pathogen load. Pathogen load can be measure by any means known in the art including, but not limited to real-time PCR, PCR, Western blot, ELISA, hemagglutinatin inhibition assay, virus neutralization assay or other serologic assays.

Another aspect of the present disclosure provides a method for preparing a cell, tissue, or an organ for transplant. In some embodiments, the method comprises contacting the cell, tissue, or organ with a composition comprising an in vitro transcribed (IVT) RNA comprising short interspersed nuclear elements (SINEs), or fragments thereof.

The cell, tissue, or organ can be any cell, tissue or organ that will be transplanted. The organ may include, without limitation heart, heart valves, lung, kidney, liver, pancreas, skin, spleen, middle ear, connective tissue, intestine, colon, eye, stomach, ovary, testes, bladder, uterus and adrenal glands. The cell population may comprise a stem cells, bone marrow and immune cells. Tissues may comprise bones tendons, ligaments, skin, heart valves blood vessels, pancreas islets, nerves, veins, and limbs.

In some embodiments, the method further comprises transplanting the article into a subject. Organ transplantation is a medical procedure in which an organ is removed from one body and placed in the body of a recipient, to replace a damaged or missing organ. The donor and recipient may be at the same location, or organs may be transported from a donor site to another location. An allograft is a transplant of an organ or tissue between two genetically non-identical members of the same species. Due to the genetic difference between the organ and the recipient, the recipient's immune system may identify the organ as foreign and attempt to destroy it, causing transplant rejection. In addition, in cases of stem cell, bone marrow or other hematopoietic transplants the immune cells of the transplant attack the host cells. This is called Graft-versus-host disease (GvHD). “Graft” refers to transplanted, or donated tissue, and “host” refers to the tissues of the recipient. Transplantation recipients often receive prophylactic treatment to suppress the immune system after the transplant. These treatments continue after transplantation. Immunosuppressant treatment includes, without limitation, Ruxolitinib, Belumosudil, Ibrutinib, corticosteroids as well as photopheresis. Human leukocyte antigen (HLA) typing or HLA matching is used to match recipients and donors for transplants. HLA are proteins found on most cells in your body and are used by the immune system to recognize foreign cells. HLA genes of the donor and recipient must be the same or match as closely as possible for transplantation to be successful and to lessen the chance of developing GvHD or transplant rejection. Two main classes of HLA antigens are recognized: HLA class I and HLA class II. HLA class I antigens (A, B, and C in humans) render each cell recognizable as “self,” whereas HLA class II antigens (DR, DP, and DQ in humans) stimulate the immune system. The methods provided herein may prepare a cell, tissue or organ for transplant such that it may reduce the need for, or the levels of, immunosuppressant treatment needed to avoid transplant rejection of GvHD or increase the likelihood of the recipient accepting the transplanted organ.

In some embodiments, the transplant recipient is administered IVT RNA comprising SINEs, or fragments thereof, prior to transplantation. The IVT RNA may be administered in vivo, for to an organ, cell or tissue ex-vivo or in vitro. In some embodiments, the subject is scheduled for cell, organ or tissue transplant, such that the subject is to receive a transplanted organ, tissue or cell.

Another aspect of the present disclosure provides a method of enhancing immunotolerance in subject in need thereof. The method may comprise, administering a composition comprising an IVT RNA comprising short interspersed nuclear elements (SINEs), or fragments thereof. In some embodiments, the subject is scheduled for cell, organ, or tissue transplant, and/or wherein the subject is immunocompromised, e.g., comprises an immunodeficiency disorder or is in an immunocompromised state. Subjects who are immunocompromised have a weakened immune system and have a reduced ability to fight infection or other disease. An immunocompromised state may be the result of another disease or condition, genetic disorder or due to treatments or medicine. An immunocompromised subject may also be called immunosuppressed. Immune tolerance, or immunological tolerance or immunotolerance, is the process by which immune cells are made unresponsive to antigens, including self-antigens to prevent damage to healthy tissues. It prevents an immune response to antigens produced by the body itself, recognized from a prior encounter or foreign tolerogenic antigens.

Another aspect of the present invention provides a method of treating wounded tissue in a subject in need thereof, the method comprising: administering to the wounded tissue an agent or composition comprising an in vitro transcribed (IVT) RNA comprising short interspersed nuclear elements (SINEs), or fragments thereof. The wound may be a thermal wound, including a radiation wound, chronic wound, acute wound or surgical wound. In some embodiments, the wound is a skin wound. In some embodiments, the wound is an organ wound, An organ wound may be in any organ, including, but not limited to lung, and eye.

In embodiment, the IVT RNA comprising the SINEs (e.g. IVT Alu RNA) or a composition comprising the IVT RNA can contain sense strands, antisense strands, or a combination thereof. The IVT RNA or composition can also comprise combinations of different Alu elements in the same or different orientations. In some embodiments, the IVT RNA comprises at least one modified nucleotide. In some embodiments, the IVT RNA sense strands, antisense strands, or combinations thereof are modified nucleotides to reduce innate immune responses. For example, the IVT RNA strands can comprises modified nucleosides, such as pseudouridine (abbreviated by the Greek letter “psi” or “ψ”), N1-methylpseudouridine, 5-methylcytosine (m5C), 5-methyluridine (m5U), 2′-O-methyluridine (Um or m2′-OU), 2-thiouridine (s2U), or N6-methyladenosine (m6A)). In some embodiments, the IVT RNA comprises all pseudouridine and no uridine. Pseudouridine substitute may reduce the innate immune response to unmodified RNA (Kariko, K. et al., Mol Ther 16:1833-1840 (2008)). In vitro transcription can be carried out by ay means known in the art. For example, in vitro transcription can be carried out with a commercially available kit with allows for the modification of nucleotides, such as the substitution of uridine for pseudouridine.

Another aspect of the present invention comprises, prior to administration, determining the level of Alu RNA in a biological sample from the subject. Determining the level of Alu RNA in a sample may include methods of measuring an amount or expression of Alu RNA. These detection methods may include methods of measuring an amount or expression of Alu RNA in tissue. The methods of detecting Alu RNA in a tissue sample include obtaining a tissue sample from a subject and measuring the amount or expression of Alu RNA in the tissue sample. Detecting Alu RNA may be performed by various methods known in the art, including techniques selected from polymerase chain reaction (PCR), reverse-transcription PCR (RT-PCR), including competitive RT-PCR, quantitative PCR (qPCR), and RT-qPCR. Alu SINEs produce short lived full length Alu (fl-Alu) transcripts, which are processed into a stable small cytoplasmic Alu (sc-Alu) RNA⁵, thus quantifying the expression of fl-Alu by some methods, for example RT-qPCR can be difficult.

Therefore, the inventors have developed a novel competitive RT-PCR method for quantifying fl-Alu and sc-Alu RNA. Full-length (fl-Alu) Alu RNA is about 300 bp. However, shorter Alu or fragments of the Alu element can also be used including fragments at least 20, 25, 30, 35, 40, 45, 50, 60, 70, 80, 90, 100, 110, 120, 130, 140, 141, 142, 143, 144, 145, 156, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, or 300 bp in length. Shorter cytoplasmic Alu (sc-Alu) RNA is about 100 bp. In some aspects, the sc-Alu is at least about 20, 25, 30, 35, 40, 45, 50, 60, 70, 80, 90, or 100 bp in length. In embodiments, the Alu comprises an insertion or deletion mutation. In some aspects, the Alu is about 100 base pairs (bp) to about 350 bp.

The novel competitive RT-PCR method for quantifying fl-Alu and sc-Alu RNA involves co-amplification from test RNA with an internal standard using common primers in a single reaction. In the present disclosure competitive RT-PCR is used to quantify the expression levels of fl-Alu and sc-Alu RNA. This assay contains a primer set that recognizes both the fl-Alu and sc-Alu (5′ CCGGGTGCGGTGGCACACGCT (SEQ ID NO: 1), and 5′-GCAATCTCCTTCTCACGGGTT, (SEQ ID NO: 2)) and will amplify the most abundant form of Alu. The primer sets may have at least 99%, 98%, 97%, 96% or 95% sequence identity to SEQ ID NOs: 1 and 2. The resulting RT-PCR products are analyzed, for example by gel electrophoresis, and the ratio of fl-Alu to sc-Alu is determined, for example by densitometry. Total RNA isolated from cells following heat shock, which has been shown to increase fl-Alu can be used as a positive control. Additionally, to exclude amplification of genomic Alu elements Alu PCR using total RNA can be used. In embodiments, methods of detecting an amount or expression of Alu RNA include measuring the amount or expression of a small cytoplasmic Alu (sc-Alu) RNA in the sample. Further, embodiments include measuring the amount to full length Alu (fl-Alu) RNA in the sample and comparing the amount of sc-Alu RNA to fl-Alu RNA.

In some embodiments, a tissue sample is obtained from a subject to detect the Alu RNA. Any tissue that may comprise the Alu RNA may be used. In some embodiments the tissue may comprise placenta, an organ, cell or tissue for transplant, a wound or wounded organ, heart, lung, blood, serum, plasma, vaginal discharge, urine, lymphatic fluids, umbilical blood or tissue, and amniotic fluid.

Detecting Alu RNA may also be performed by techniques for visualizing Alu RNA in tissue, such as by in-situ hybridization. In situ hybridization can be used to visualize Alu RNA in tissue. In situ hybridization (ISH) is a technique that allows the detection and localization of viral nucleic acid (DNA or RNA) in tissue sections or cytological specimens using labelled nucleic acid probes with complementary sequences to the target viral nucleic acid. To visualized Alu RNA in tissues, in situ hybridization can be performed using a locked nucleic acid (LNA) probe (5′CACTGCACTCCAGCCTG) (SEQ ID NO: 3) designed to recognize Alu RNA transcripts, or a scrambled control. The probe may have at least 99%, 98%, 97%, 96% or 95% sequence identity to SEQ ID NO: 3. To confirm that the Alu probe recognizes Alu RNA and not Alu elements in the genomic DNA, tissue sections can be pre-treated with either RNase A or DNase I.

In situ hybridization probes can be modified for different methods of detection, including fluorescent detection, and labelled with biotin or digoxigenin and other means known in the art. Visualization of Alu SINE RNA with ISH can also be co-localized by using probes designed for other possible co-localization targets. For example, an Alu SINE RNA probe can be used with a probe for a C19MC miRNA to determine if the Alu SINE RNA and miRNA co-localize.

In some embodiments, a tissue sample is obtained from a subject to detect the Alu RNA. Any tissue that may comprise the Alu RNA may be used. In some embodiments the tissue may comprise placenta, heart, and lung or cells within them, such as syncytiotrophoblast cells of the placenta. In some embodiments, the tissue may be obtained from a tissue biopsy, for example liver, breast, thyroid, bladder, brain and skin biopsy. The tissue may also be obtained from a subject exposed to an infection. The tissue may also be obtained from a subject undergoing organ transplantation. In some embodiments, an uninfected, non-diseased or control tissue may also be used.

A subject, as used herein, may comprise a mammalian subject including a fetus or fetal tissue in utero. The subject may be a fetus exposed to a potentially infected mother, or a human with a suspected viral infection. Alu SINEs are specific to human and non-human primates. Therefore, “subject” as used herein may refer to human and non-human primates without being confined to any particular sex, age, and/or species. A subject may also comprise those with a disease or condition or those suspected of having a disease or condition with abnormal regulation of C19MC miRNA genes. For example, a subject having, or suspected of having preeclampsia, preterm birth, intrauterine growth restriction and cancers such as hepatocellular carcinoma, breast cancer, parathyroid tumor, brain cancer, lung cancer, bladder cancer, infantile hemangioma, and infant brain cancers. A subject may also be in need of an organ transplant, or be in need of enhancing immunotolerance. A subject may have a wound, including a organ or tissue wound. Additionally, a subject may be suspected of having, or have abnormal embryonic development. The term “subject” may be used interchangeably with the terms “individual” and “patient” and includes human and non-human mammalian subjects

### Additional Definitions

The disclosure has been described in terms of one or more preferred and exemplary embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or Figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

“Percentage of sequence identity” or “percent similarity” is determined by comparing two optimally aligned sequences over a comparison window, wherein the portion of the polynucleotide or peptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “substantial similarity” of polynucleotide or peptide sequences means that a polynucleotide or peptide comprises a sequence that has at least 75% sequence identity. Alternatively, percent identity can be any integer from 75% to 100%. More preferred embodiments include at least: 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

“Substantial identity” of amino acid sequences for purposes of this invention normally means polypeptide sequence identity of at least 75%. Preferred percent identity of polypeptides can be any integer from 75% to 100%. More preferred embodiments include at least 75%, 80%, 85%, 86%, 87%, 88%, 89%, 90%, 91%, 92%, 93%, 94%, 95%, 96%, 97%, 98%, 98.7%, or 99%.

The invention will be more fully understood upon consideration of the following non-limiting examples.

The present invention has been described in terms of one or more preferred embodiments, and it should be appreciated that many equivalents, alternatives, variations, and modifications, aside from those expressly stated, are possible and within the scope of the invention.

The following Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### Exemplary Embodiments

Embodiment 1. A method for reducing the likelihood of amniotic fluid infection and/or fetal infection, or treating amniotic fluid infection and/or fetal infection, the method comprising: vaginally administering to a pregnant female a composition comprising an in vitro transcribed (IVT) RNA comprising short interspersed nuclear elements (SINEs), or fragments thereof.

Embodiment 2. The method of embodiment 1, wherein the administration is topical.

Embodiment 3. The method of any of the previous embodiments, wherein the composition is in the form of a cream, lotion, gel, or suppository or insert.

Embodiment 4. The method of any of the previous embodiments, wherein the infection is diagnosed as viral.

Embodiment 5. The method of any of the previous embodiments, wherein the infection is diagnosed as bacterial.

Embodiment 6. The method of any of the previous embodiments, wherein the infection is diagnosed as fungal.

Embodiment 7. A method for preparing a cell, tissue, or an organ for transplant, the method comprising: contacting the cell, tissue, or organ with a composition comprising an in vitro transcribed (IVT) RNA comprising short interspersed nuclear elements (SINEs), or fragments thereof.

Embodiment 8. The method of embodiment 7, wherein the transplant recipient is administered IVT RNA comprising SINEs, or fragments thereof, prior to transplantation.

Embodiment 9. A method of enhancing immunotolerance in subject in need thereof, the methods comprising: administering a composition comprising an in vitro transcribed (IVT) RNA comprising short interspersed nuclear elements (SINEs), or fragments thereof, optionally, wherein the subject is scheduled for cell, organ, or tissue transplant, and/or wherein the subject is immunocompromised, e.g., comprises an immunodeficiency disorder.

Embodiment 10. A method of treating wounded tissue in a subject in need thereof, the method comprising: administering to the wounded tissue a composition comprising an in vitro transcribed (IVT) RNA comprising short interspersed nuclear elements (SINEs), or fragments thereof.

Embodiment 11. The method of embodiment 10, wherein the wound is a skin wound, and the composition is administered topically.

Embodiment 12. The method of embodiment 10, wherein the wound is in lung tissue, and the composition is administered via inhalation.

Embodiment 13. The method of embodiment 10, wherein the wound is an eye wound, and the composition is administered topically to the wounded eye.

Embodiment 14. The method of any of the previous embodiments, wherein the IVT RNA comprises at least one modified nucleotide.

Embodiment 15. The method of embodiment 14, wherein at least one modified nucleotide comprises one of pseudouridine, NI-methylpseudouridine, 5-methylcytosine (m5C), 5-methyluridine (m5U), 2′-O-methyluridine (Um or m2′OU), 2-thiouridine (s2U), or N6-methyladenosine (m6A)).

Embodiment 16. The method of any of embodiments 1, 7, 9, or 10, wherein the SIINE comprises Alu repeats.

Embodiment 17. The method of any of embodiments 14-16, wherein the SINE comprises AluJ, AluS, and/or ALuY RNA sense strands, antisense strands, or a combination thereof.

Embodiment 18. The method of any one of embodiments 1, 7, 9, or 10, wherein the IVT RNA comprises at least one psuedouridine.

Embodiment 19. The method of any one of embodiments 14-18, wherein the IVT RNA comprises all psuedouridine and no uridine.

Embodiment 20. The method of any one of embodiments 1, 7, 9, or 10, comprising, prior to administration, determining the level of Alu RNA in a biological sample from the subject.

Embodiment 21. The method of any of the previous embodiments, wherein detecting the level of Alu RNA comprises measuring the level of a small cytoplasmic Alu (sc-Alu) RNA in the sample.

Embodiment 22. The method of embodiment 20 or 21, wherein the method comprises measuring the level of full length Alu (fl-Alu) RNA in the sample, and optionally, comparing the amount of sc-Alu RNA to fl-Alu RNA.

Embodiment 23. The method of embodiment 22, wherein the fl-Alu RNA and sc-Alu RNA are detected with primers comprising SEQ ID NO: 1 and SEQ ID NO: 2.

Embodiment 24. The method of embodiment 20, wherein the tissue sample is from a tissue or organ biopsy, placenta, blood, serum, plasma, vaginal discharge, urine, lymphatic fluids or amniotic fluid.

### EXAMPLES

Primates and rodents have hemochorial placentas with heightened risk of vertical viral transmission. In the following examples, the inventors uncovered a convergently evolved mechanism by which primate-specific Alu and rodent-specific B1 SINE RNA of the C19MC and the C2MC miRNA clusters, respectively, drive their placental constitutive type III interferon expression and antiviral protection.

### Example 1 Transcriptional Activation of C19MC Induced a Strong Type III IFN Response

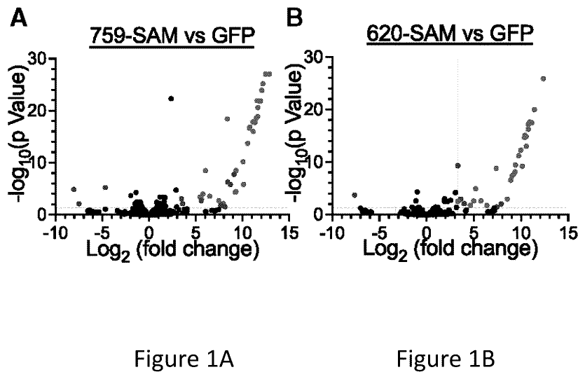

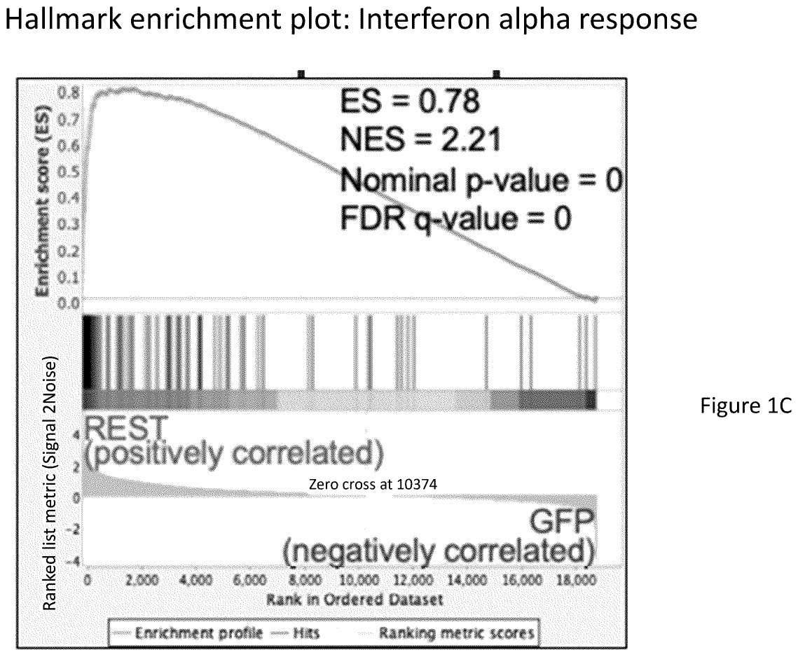

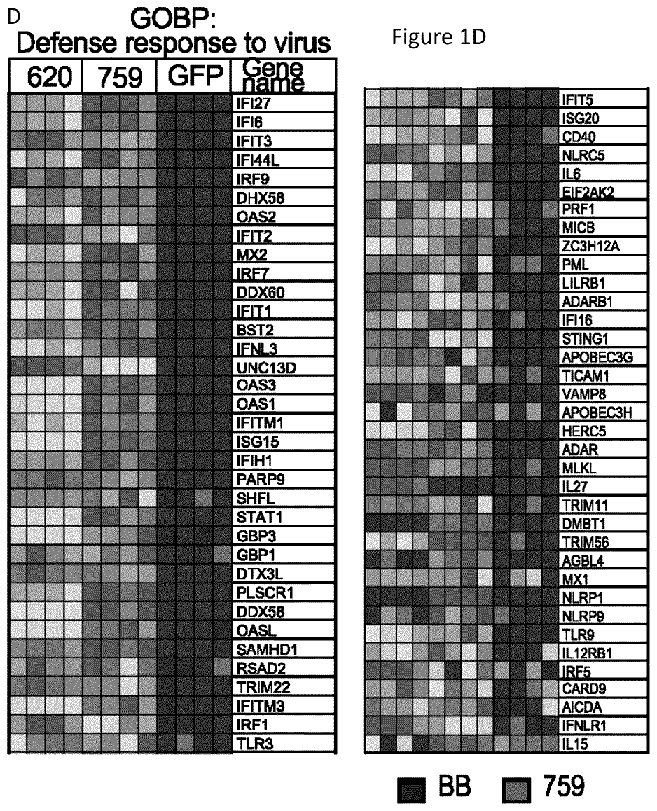

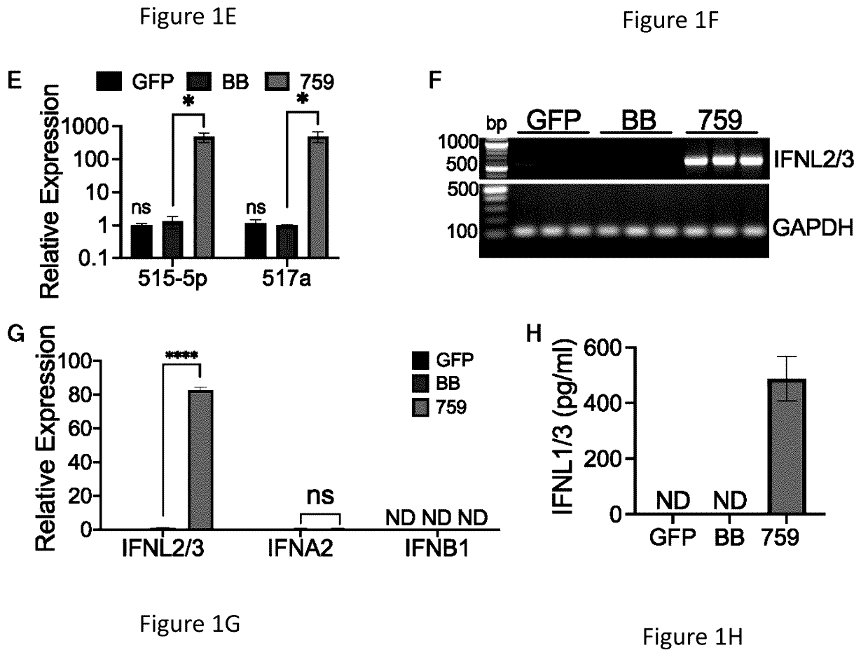

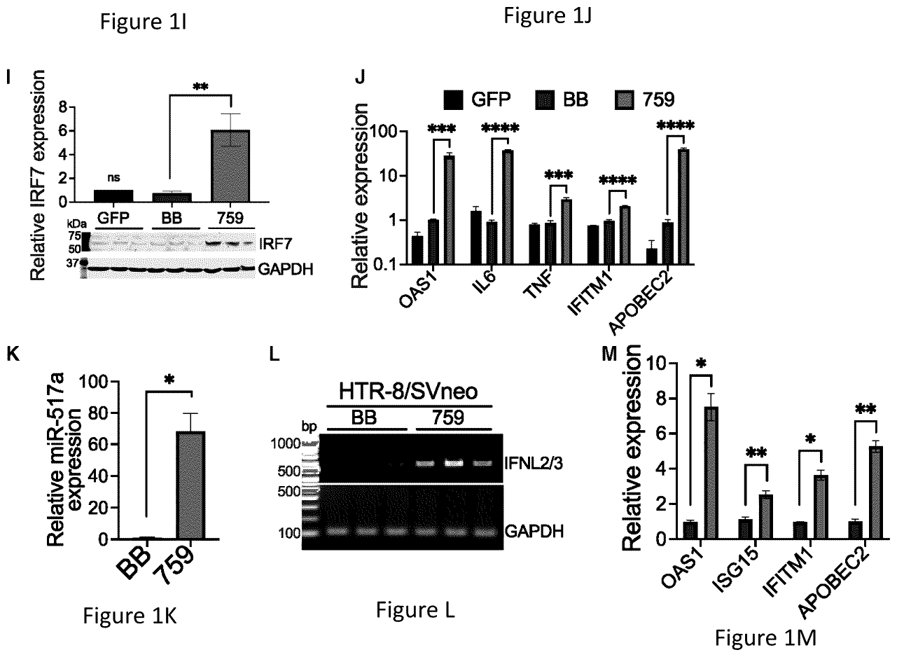

In the human placenta, the C19MC gene is transcribed by RNA polymerase II (RNA Pol II) as a single, large, non-protein-coding transcript from the positive strand of the genomic DNA.20 To investigate the role of the C19MC Alu SINEs in the placenta and mimic the RNA Pol II transcriptional activation of C19MC, our previously established CRISPR-dCas9 synergistic activation mediator (SAM) system and two different single guide RNAs (sgRNAs), the 620 and the 759, were used to transcriptionally activate the entire cluster.21 Small RNA sequencing (sRNA-seq) analysis of AD-293 cells transfected with 759-SAM or 620-SAM for 72 h displayed an increase (>10-fold, adjusted p<0.05) in the expression of 38 major-strand mature miRNAs, 30 of which belong to C19MC, compared with control green fluorescent protein (GFP)-transfected cells (FIG. 1A; Table 1), whereas 620-SAM-transfected AD-293 cells displayed up-regulation in 33 major-strand miRNAs, 28 of which belong to C19MC (FIG. 1B; Table 1). On the other hand, only three and one miRNAs that do not belong to the C19MC were down-regulated in 759-SAM and 620-SAM-transfected cells, respectively (FIGS. 1A and 1B; Table 1). Thus, the transcriptional activation of C19MC by 759-SAM increases the expression of C19MC with high specificity.

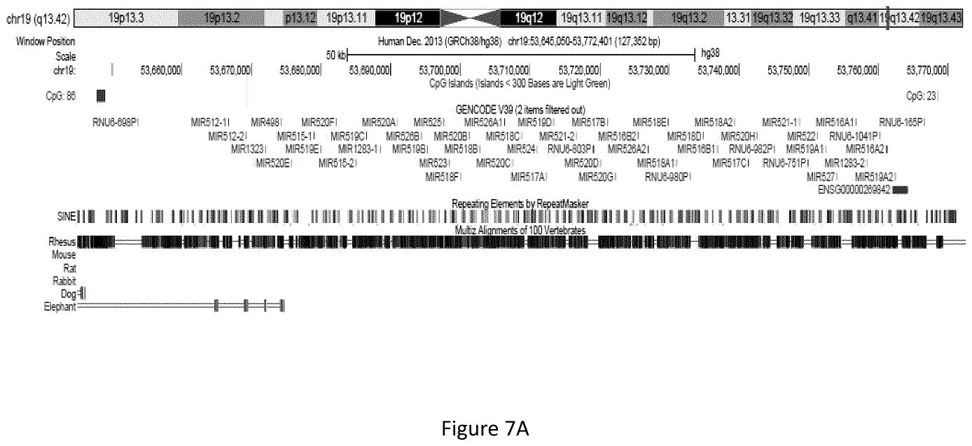

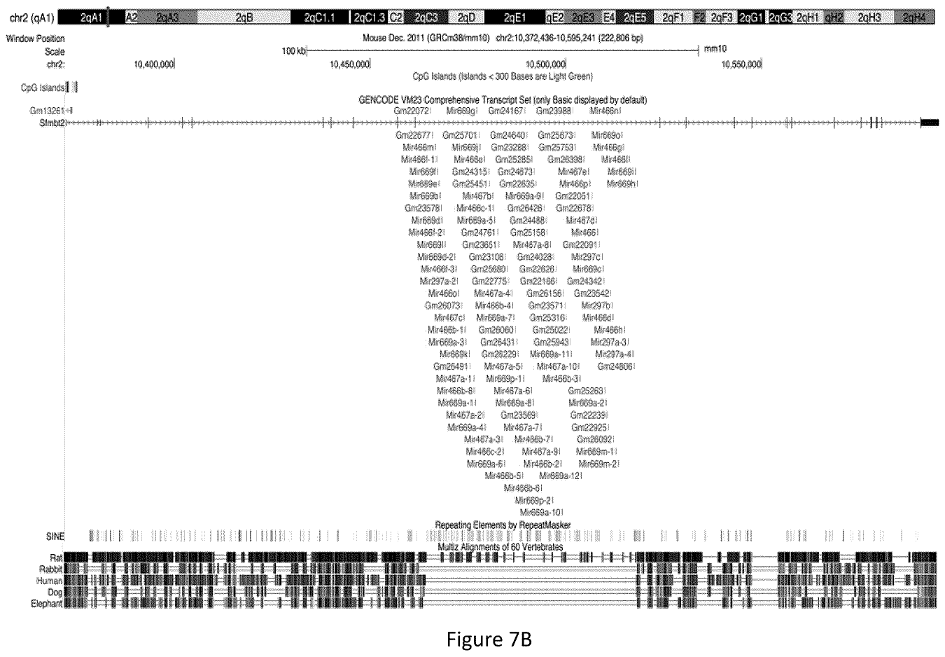

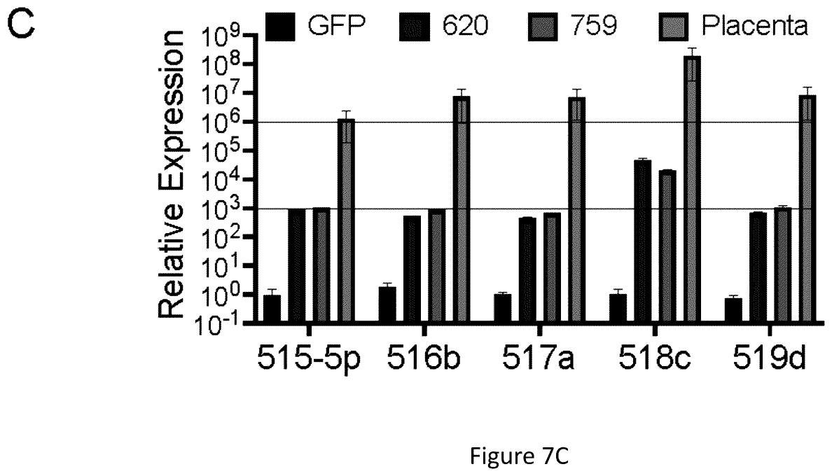

Although AD-293 cells transfected with 759-SAM and 620-SAM showed approximately 103-fold increase in the expression of selected C19MC miRNAs by RT-qPCR, the expression of these miRNAs in the term human placenta was >106-fold higher than in GFP-transfected cells (FIG. 7C).

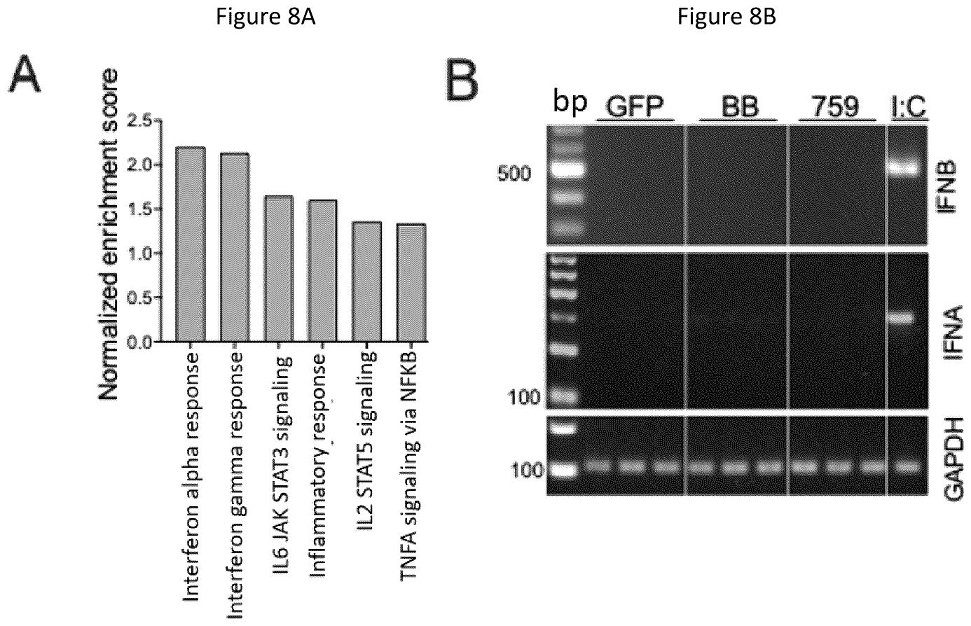

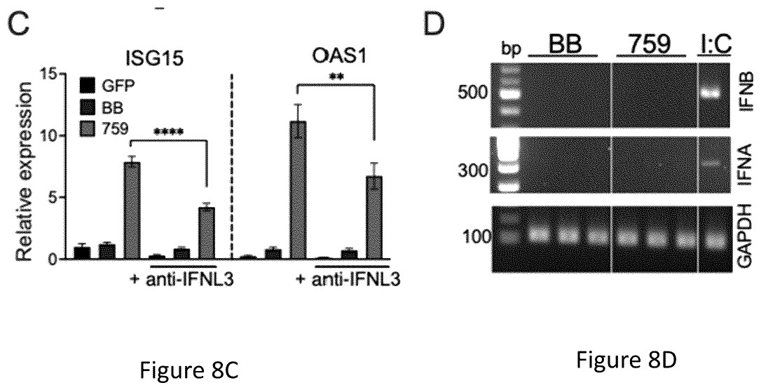

To identify the regulatory pathways affected by C19MCactivation, RNA-seq and then gene set enrichment analysis (GSEA) were performed, and the results show IFNα, IFNγ, and inflammatory and defense response to virus among the most enriched in both 759-SAM- and 620-SAM-transfected cells compared with control GFP (FIGS. 1C, 1D, and 8A). However, the RNA-seq data showed a significant increase in the expression of not type I or type II IFNs but rather type III IFN in both 759-SAM and 620-SAM-transfected AD-293 cells compared with control GFP (IFNL3 and IFNL2 exhibited 515-fold increase, p=5.4 3 10_12, and 420-fold, p=1.2 3 10_11, respectively, in 759-SAM-transfected cells, and 104-fold, p=4.8 3 10_7, and 76-fold, p=2.1 3 10_6, respectively, in 620-SAM-transfected cells) and numerous ISGs (FIG. 1D; Table 2). Although type I, II, and III IFNs bind to different cell surface receptors, their downstream signaling transduction pathways overlap. Thus, in addition to the activation of their specific genes, they also activate a common set of ISGs. Because the only curated hallmark gene sets available for IFNs in the GSEA platform are for IFNα and IFNγ, the increase in IFNL2 and IFNL3 may be responsible for the enrichment of IFNα and IFNγ gene sets in AD-293 cells transfected with 759-SAM and 620-SAM compared with control GFP.

Because 759-sgRNA binds at two locations upstream of the first two miRNAs of C19MC, ~2-fold higher expression of the C19MC miRNAs and higher number of differentially expressed genes was observed compared with 620-sgRNA that binds to only one location upstream of the first C19MC miRNA.21 Thus, in subsequent experiments, 759-SAM was used. To ensure that the 759-SAM-mediated IFNL induction is not due to the presence of the CRISPR/SAM components, AD-293 cells were transfected with backbone (BB) control sgRNA and all the CRISPR/SAM components (BB-SAM). To confirm the increase in the expression of C19MC, we performed RT-qPCR for two randomly selected C19MC miRNAs, miR-515-5p and miR-517a, and found >450-fold increase in the expression of these miRNAs in the 759-SAM-transfected cells, whereas the BB-SAM-transfected cells were similar to control GFP (FIG. 1E). Because IFNL2 and IFNL3 share >96% sequence homology, we used a primer set that recognizes both (IFNL2/3) and found that compared with control BB-SAM- or GFP-transfected AD-293 cells, transfection with 759-SAM-transfected cells showed an increase in IFNL2/3 by RT-PCR (FIG. 1F), RT-qPCR (FIG. 1G), and ELISA for IFNL1/3 of the conditioned media (FIG. 1H), whereas type I INFs were not detected by RNA-seq (Table 2), RT-qPCR (FIG. 1G), or RT-PCR (FIG. 8B).

Moreover, IFNL autocrine/paracrine signaling was also observed with the significant increase in the expression of several ISGs in 759-SAM-transfected cells, including IRF7, by immunoblot (FIG. 1I) and OAS1, IL6, TNF, IFITM1, and APOBEC2 by RT-qPCR (FIG. 1J). Importantly, the addition of anti-IFNL3 antibody to the supernatant of AD-293 cells transfected with 759-SAM significantly reduced the expression of ISG15 and OAS1 by ~50%, confirming IFNL3 autocrine/paracrine signaling (FIG. 8C).

To confirm that C19MC mediates the constitutive IFNL and ISG expression in the trophoblast, we transfected HTR-8/SVneo cells, which are immortalized human first-trimester extravillous trophoblasts (EVTs) that do not express C19MC, with 759-SAM or control BB-SAM. Similar to AD-293 cells, 759-SAM-transfected HTR-8/SVneo cells also showed a significant increase in the expression of miR-517a (FIG. 1K), IFNL2/3 (FIG. 1L), and ISGs, including OAS1, ISG15, IFITM1, and APOBEC2 (FIG. 1M), compared with BB-SAM-transfected cells, whereas type I IFNs, such as IFNα (IFNA) and IFNβ (IFNB), were not detected (FIG. 8D). Thus, even in the absence of viral infections, the transcriptional activation of the C19MC induces type III IFN and its downstream ISGs.

### Example 2 Activation of C19MC Preferentially Induces IFNL and ISGs in a miRNA-Independent Fashion

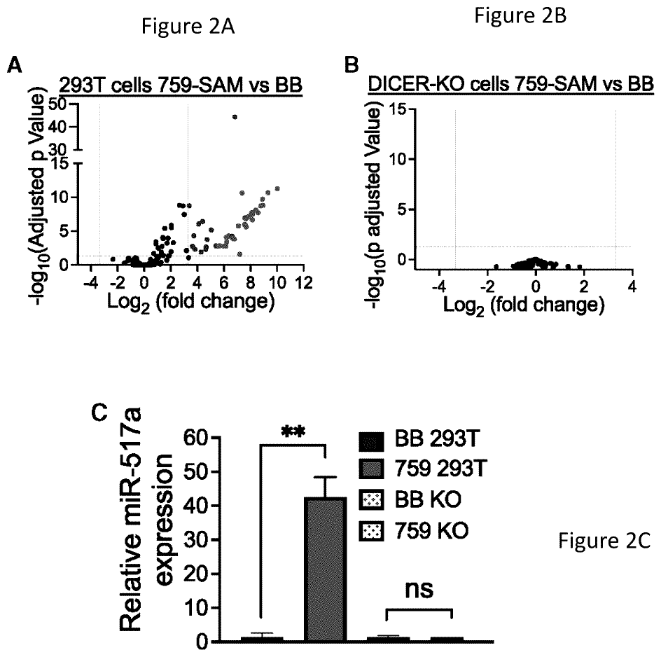

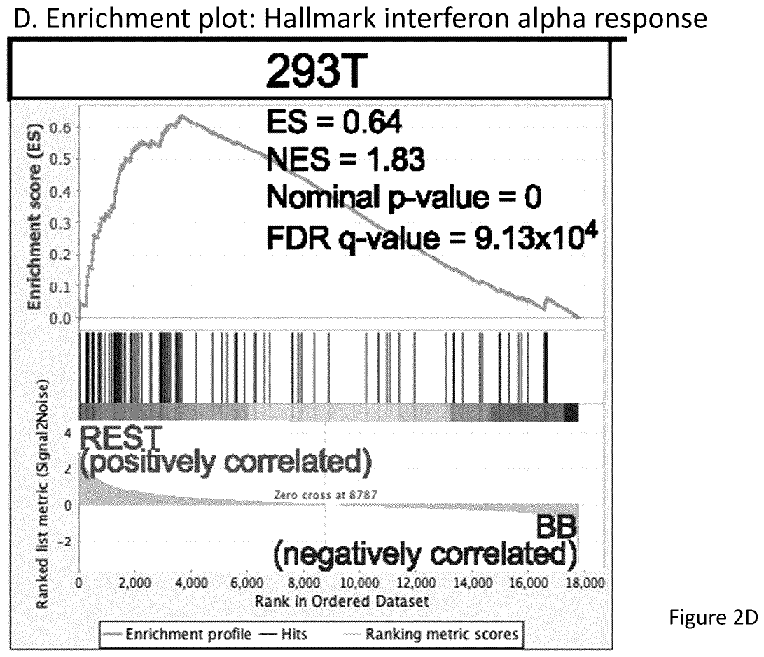

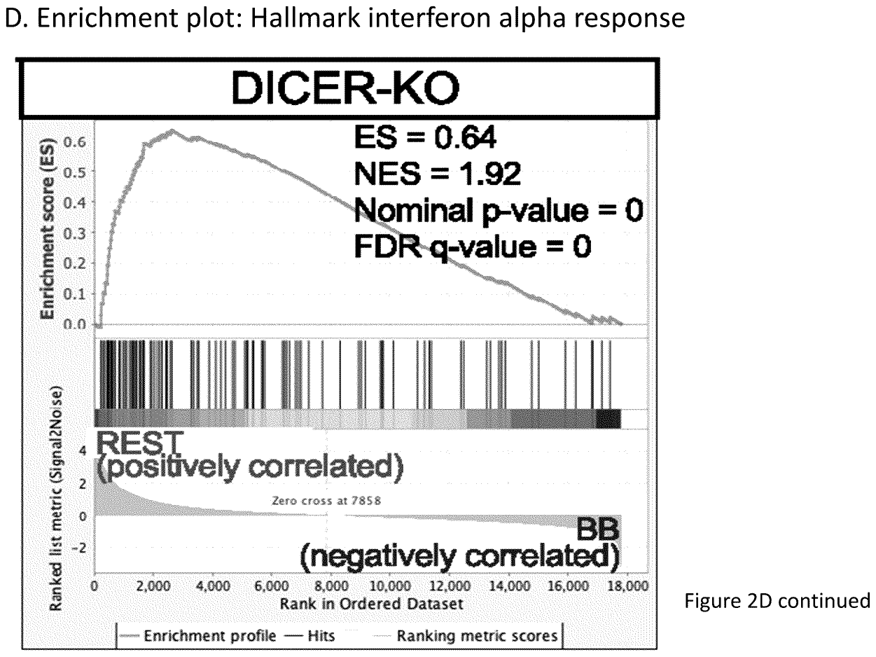

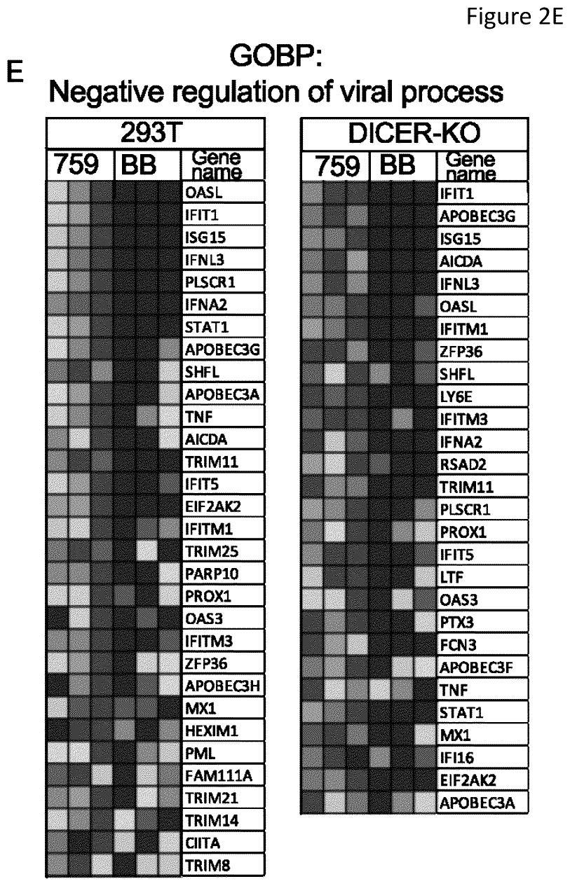

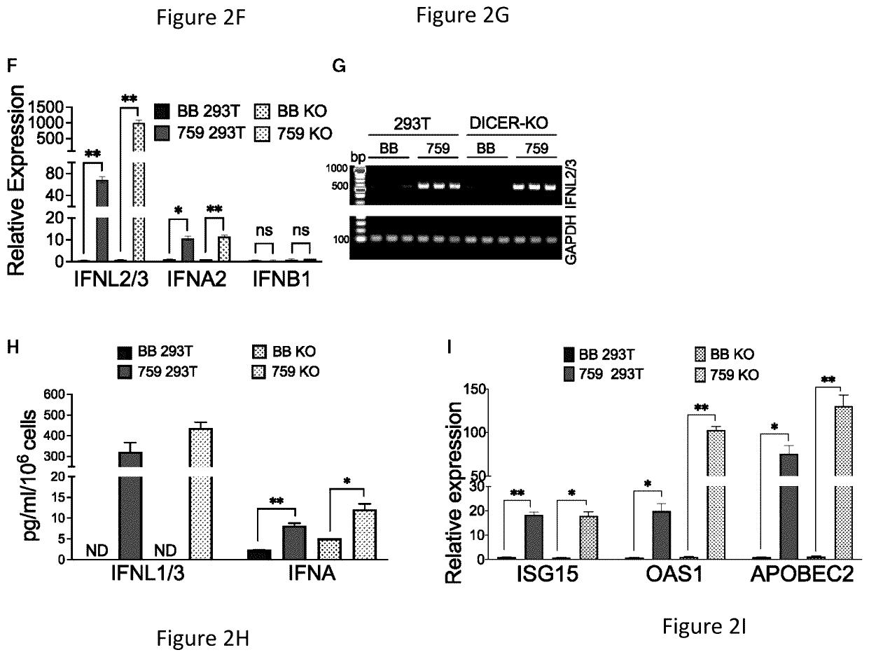

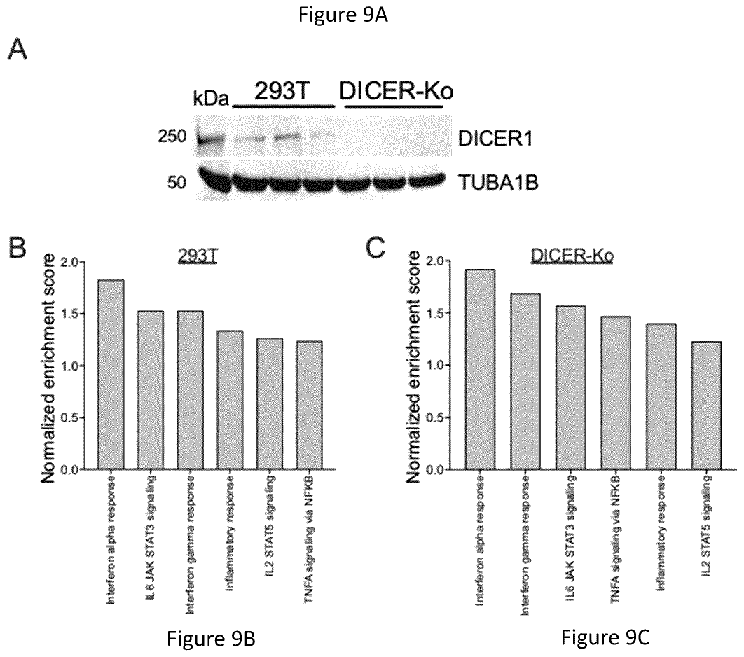

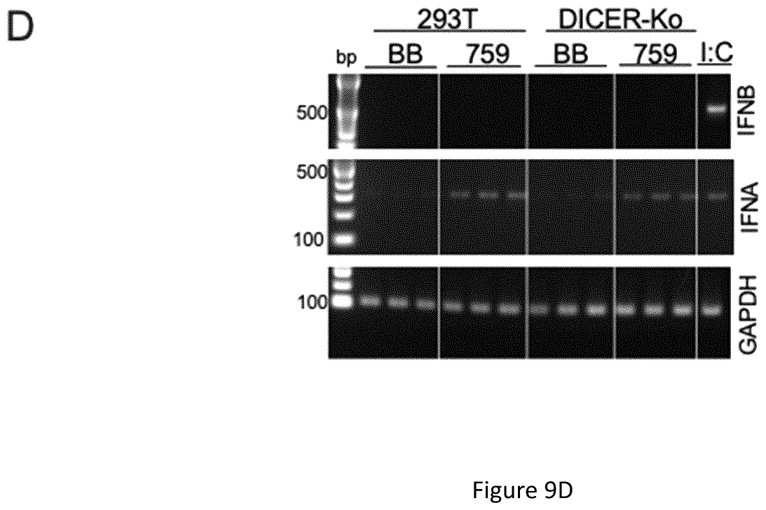

To investigate the role of C19MC SINEs and distinguish C19MC SINEs from the C19MC miRNAs, we used the 759-SAM system to transcriptionally activate C19MC in DICER1 knockout 293T cells (DICER-KO) and control wild-type (WT) 293T (FIG. 9A). SRNA-seq analysis (FIGS. 2A and 2B; Table 1) and RT-qPCR (FIG. 2C) showed a significant increase (>10-fold, adjusted p<0.05) in the expression of 44 major-strand mature miRNAs, 29 of which belong to C19MC, compared with control BB-SAM-transfected cells, whereas in DICER-KO cells, no miRNAs were detected with significant expression changes. Importantly, hallmark GSEA of 759-SAM compared with BBSAM-transfected cells showed IFNα, IFNγ, inflammatory response, and the negative regulation of viral process among the most enriched in both DICER-KO and 293T cells (FIGS. 2D, 2E, 9B, and 9C). Specifically, the expression of IFNL3 was significantly increased in both DICER-KO and 293T cells transfected with 759-SAM, as evaluated by RNA-seq (FIG. 2E; Table 3), RT-qPCR (FIG. 2F), RT-PCR (FIG. 2G), and ELISA of the conditioned media (FIG. 2H), as well as several ISGs (FIGS. 2E and 21) compared with BB-SAM. Unlike 759-SAM transfected AD-293 and HTR-8/SVneo cells, in which type I IFN was not induced, 293T and DICER-KO cells showed a slight increase in the expression of IFNA2 by RT-qPCR (FIG. 2F) and IFNA by RT-PCR (FIG. 9D) and ELISA of the conditioned media (FIG. 2H), whereas IFNB was not induced (FIGS. 2F, 2H, and 9D). These results demonstrate that even in the absence of viral infections, the transcriptional activation of C19MC preferentially induces type III IFNs and ISGs in a miRNA-independent manner.

### Example 3 Activation of C19MC Protects Against Viral Infection in a miRNA-Independent Manner

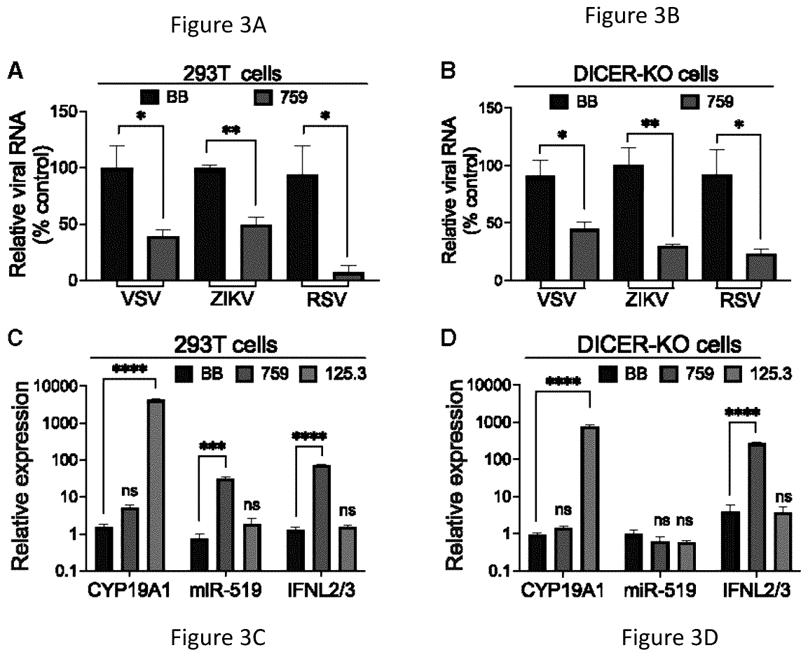

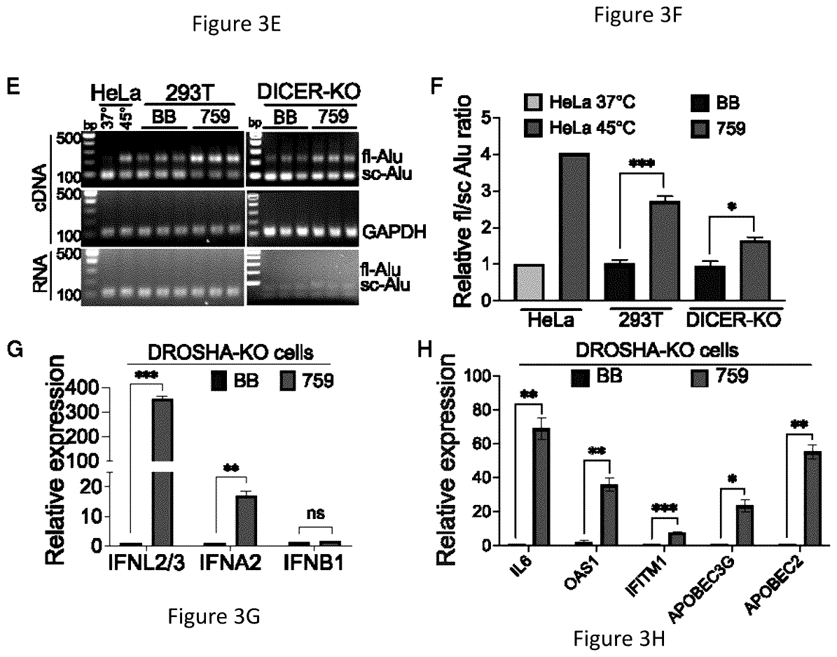

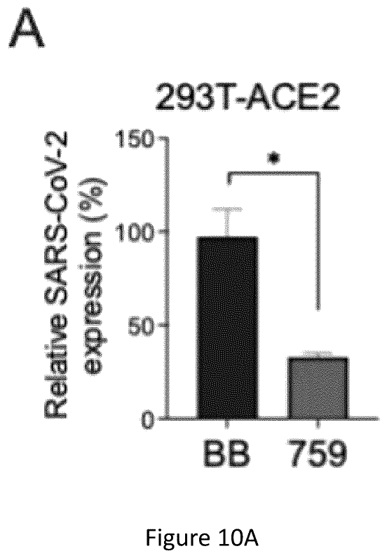

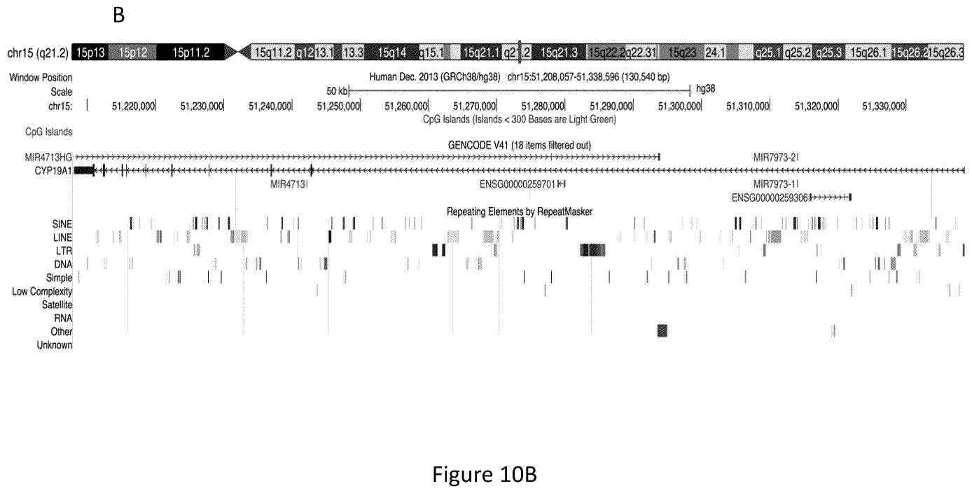

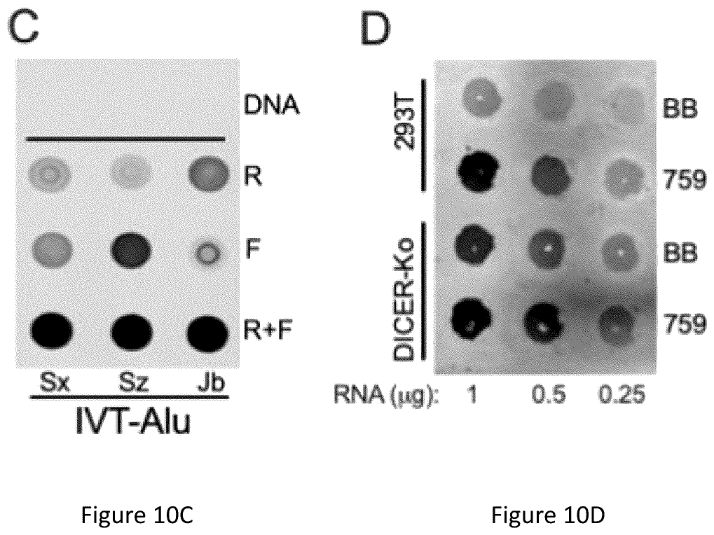

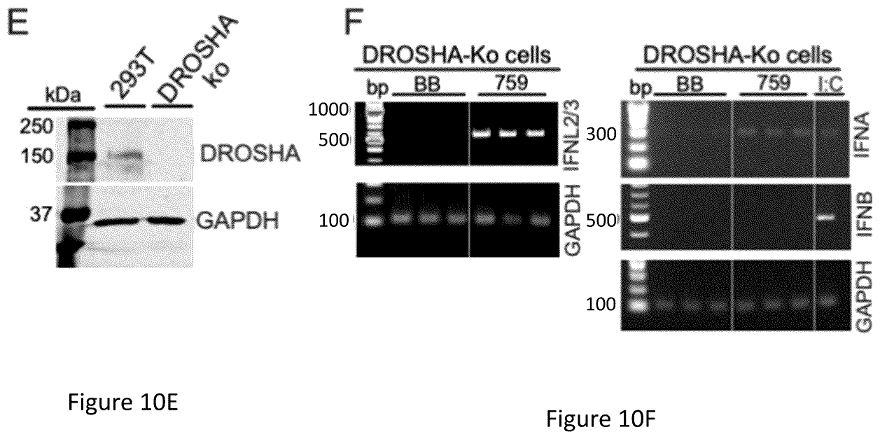

IFNs are the first line of defense against viral infections. Previous reports have shown that bacterial-artificial-chromosome-mediated overexpression of C19MC or overexpression of select C19MC miRNAs (miR517-3p, miR516b-5p, and miR512-3p) exerts antiviral activity, independent of IFNL signaling.22,23 To test whether the 759-SAM-mediated activation of C19MC, which induces IFNL production, also confers viral resistance in a miRNA independent manner, we transfected 293T and DICER-KO cells with 759-SAM or control BB-SAM, followed by infection with vesicular stomatitis virus (VSV), ZIKV, or respiratory syncytial virus (RSV). In agreement with previous reports, 23 293T cells transfected with 759-SAM showed a significant reduction (>50%, p<0.05) in the replication of all the tested viruses (FIG. 3A). We also tested the ability of C19MC activation by 759-SAM to resist infection by severe acute respiratory syndrome coronavirus 2 (SARS-COV-2) in 293T cells that expressed the SARSCOV-2 entry receptor angiotensin-converting enzyme 2 (ACE2) and found that 759-SAM-transfected 293T-ACE2 cells exhibited significant inhibition (>65%, p<0.05) of SARS-COV-2 replication (FIG. 10A). Strikingly, DICER-KO cells transfected with 759-SAM also showed significant inhibition (>50%, p<0.05) of VSV, ZIKV, and RSV replication (FIG. 3B). These results reveal that C19MC, which mainly induces type III IFN, provides antiviral protection independent of the miRNAs.

### Example 4 C19MC Alu SINEs Form dsRNA and Induce IFNL

The human genome contains ~1 million copies of Alu SINEs embedded in the positive and the negative strands of the genomic DNA near or within coding and noncoding genes, which can be transcribed by RNA Pol II. Additionally, Alu SINEs contain internal RNA Pol III promoters, and under stressful conditions, they are transcribed independently to produce short-lived, full length Alu (fl-Alu) transcripts, which are processed into a stable small cytoplasmic Alu (sc-Alu) RNA.24 The C19MC is a long noncoding RNA that encompasses primary miRNA (pri-miRNA) and Alu SINEs embedded with an antisense bias. It is transcribed by RNA Pol II from an upstream promoter on the positive genomic DNA strand as a single, large transcript that is quickly spliced and processed by DROSHA and DICER1.20,25 Because approximately 50% of the C19MC sequence consist of Alu SINEs, they are also transcribed and processed together with the C19MC pri-miRNA.

To test whether the increase in IFNL and ISGs through the transcriptional activation of C19MC is due to the increase in the C19MC Alu transcripts, we designed sgRNA (125.3-sgRNA) to transcriptionally activate the placenta-specific Cytochrome P450 Family 19 Subfamily A Member 1 (CYP19A1) gene, which also spans over 100 kb but contains only 19 Alu SINEs (FIG. 10B; Table 4). Although 293T and DICER-KO 293T cells transfected with 125.3-SAM exhibited _4,000- and _700-fold increase in CYP19A expression, respectively, they failed to induce IFNL2/3, whereas 759-SAM-transfected cells increased IFNL2/3 by 73- and 272-fold, respectively, compared with control BB-SAM (FIGS. 3C and 3D). Thus, the transcriptional activation of the Alu-rich C19MC, and not the activation of a large transcript, is responsible for the induction of IFNL2/3.

To test whether the RNA-Pol-II-mediated transcriptional activation of C19MC by 759-SAM increases Alu RNA levels, we performed competitive RT-PCR. This assay is performed with a primer set that recognizes both fl-Alu and sc-Alu irrespective of their transcription by RNA Pol II or Pol III; thus, it amplifies the most abundant Alu form. The resulting RT-PCR products are analyzed by gel electrophoresis, and the ratio of fl-Alu to sc-Alu is determined by densitometry and normalized to GAPDH. We also performed Alu RT-PCR on total RNA isolated from HeLa cells after heat shock recovery, which has been shown to increase the expression of fl-Alu through RNA Pol III,26 as a technical positive control. To exclude the amplification of genomic Alu SINEs, we also performed Alu PCR using total RNA. Under steady-state conditions, such as HeLa cells at 37_C, most of the endogenous Alu RNAs are processed into sc-Alu; thus, the competitive Alu RT-PCR showed very low fl-Alu to sc-Alu ratio (FIGS. 3E and 3F). However, upon heat shock exposure, fl-Alu is induced, and it competes on the primers, thus increasing the fl-Alu to sc-Alu ratio (FIGS. 3E and 3F). Importantly, 293T and DICER-KO cells transfected with 759-SAM showed increase in the fl-Alu to sc-Alu ratios by _2.7-fold (p<0.001) and _1.6-fold (p<0.05), respectively, compared with BB-SAM, whereas total RNA showed no fl-Alu PCR product (FIGS. 3E and 3F).

To confirm that the RT-PCR products are indeed derived from Alu RNA, we cloned and sequenced the fl-Alu RT-PCR products of heat-shocked Hela cells (n=41) and 759-SAM-transfected 293T (n=34) and DICER-KO (n=33) cells and found that all the clones aligned with Alu SINEs (Table 5).

To test whether Alu RNA forms double-stranded RNA (dsRNA), we in vitro transcribed (IVT) three members of the C19MC Alu subfamilies, AluJb, AluSx, and AluSz. Because C19MC Alu SINEs are embedded in the forward and reverse orientations, we IVT them in the forward and reverse directions and performed dot blot analysis with J2 dsRNA-specific antibody. We found that IVT Alu RNAs in the forward and reverse directions formed dsRNA when blotted separately as well as when blotted together (FIG. 10C). We also performed dot blot analysis of 759-SAM transfected 293T and DICER-KO cells and found an increase in dsRNA compared with BB-SAM (FIG. 10D). Although BBSAM-transfected DICER-KO cells exhibited higher levels of dsRNA compared with BB-SAM-transfected 293T cells, only 759-SAM-transfected and not BB-SAM-transfected DICER-KO cells induced IFNL (FIGS. 2F-2H). These results suggest that an increase in Alu RNA, and not the accumulation of pre-miRNA, induces IFNL, ISGs, and antiviral protection. To further confirm these findings, we used DROSHA-KO 293T cells (FIG. 10E). We observed the induction of IFNL2/3 (FIGS. 3G and 10F) and several ISGs (FIG. 3H) only in 759-SAM-transfected but not in BB-SAM-transfected DROSHA-KO cells, whereas IFNA2 was only slightly induced (FIGS. 3G and 10F). Taken together, our results indicate that the accumulation of C19MC Alu RNA, and not primary or precursor miRNAs, is responsible for IFNL and ISG induction.

### Example 5 C19MC Alu dsRNA Preferentially Induces Type III IFN Through RLR and PKR Signaling Pathways

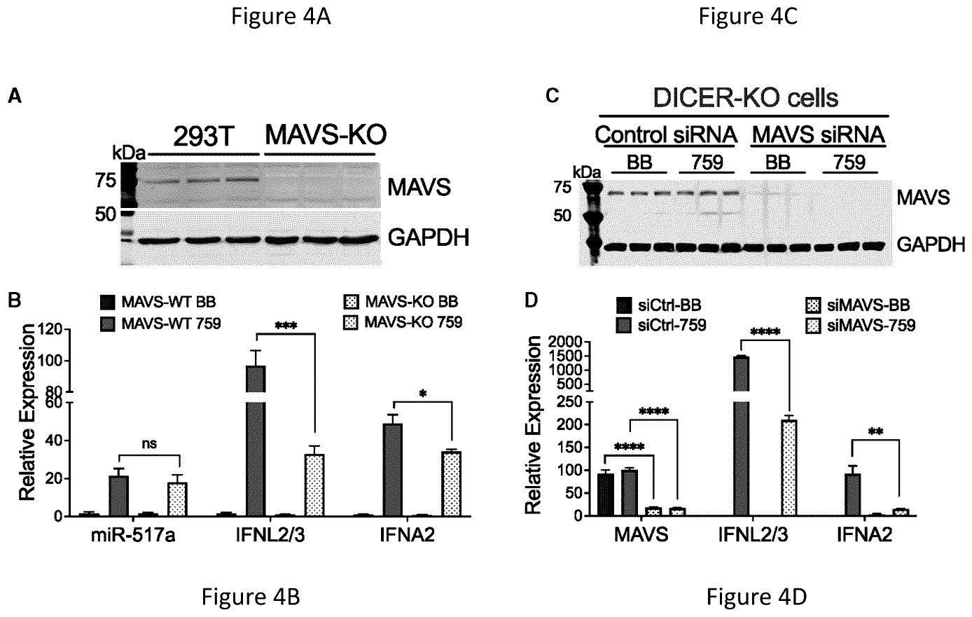

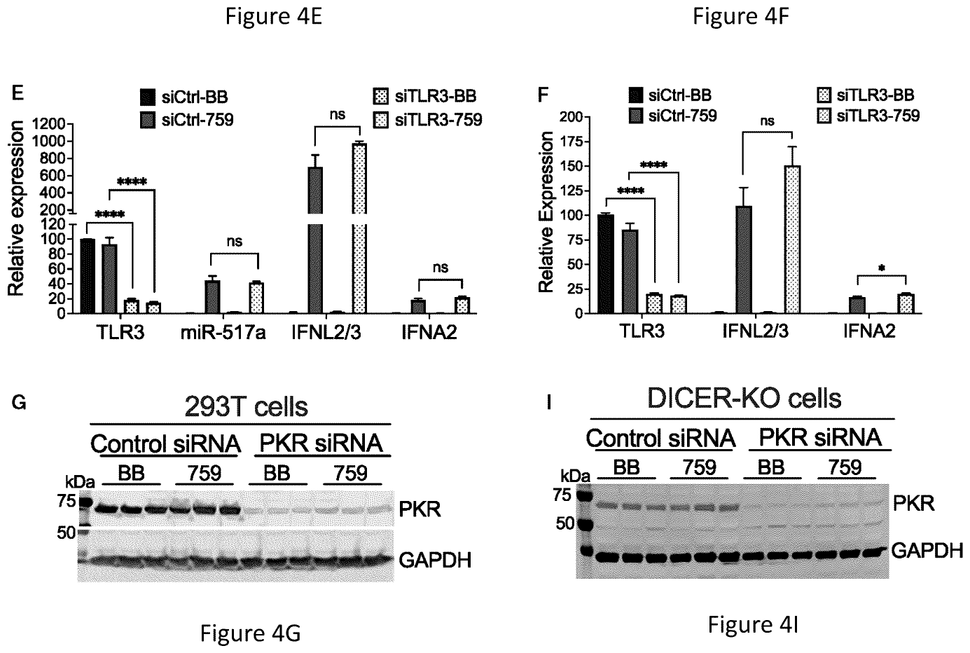

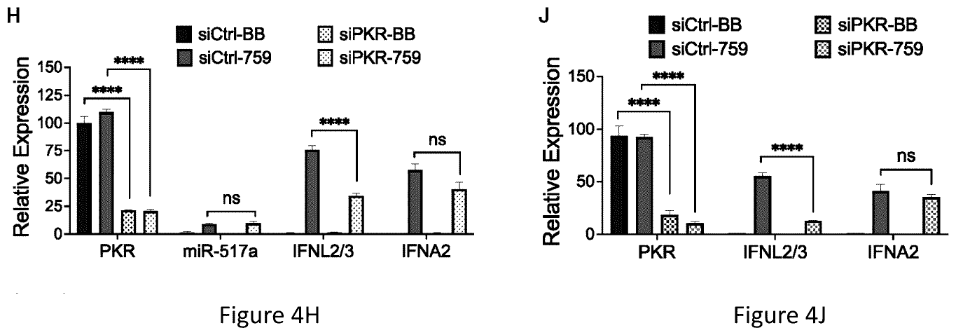

To identify the PRR responsible for the IFN induction by C19MC Alu dsRNA, we first investigated the role of the mitochondrial antiviral signaling protein (MAVS) that acts downstream of the RLRs DDX58/RIG-I, IFIH1/melanoma differentiation associated gene 5 (MDA5), and DHX58/laboratory of genetics and physiology 2 (LGP2), which recognize intracellular dsRNA and subsequently induce type I and type III IFNs.4 Thus, we used MAVS-KO 293T cells (FIG. 4A) and found that although 759-SAM increased the expression of miR-517a to levels similar to 293T cells (FIG. 4B), the expression of IFNL2/3 and IFNA2 (FIG. 4B) were significantly lower in MAVS-KO cells. To verify whether C19MC miRNA or Alu dsRNA is mediating the RLR-dependent IFN induction, we transfected DICER-KO cells with MAVS siRNA that significantly reduced MAVS expression assessed by immunoblotting (FIG. 4C) and RT-qPCR (FIG. 4D). Although we could not assess the levels of mature C19MC miRNAs in the 759-SAM-transfected DICER-KO cells, si-MAVS and 759-SAM-transfected DICER-KO cells exhibited significant reduction in IFNL2/3 and IFNA2 expression compared with si-control and 759-SAM-transfected cells (FIG. 4D).

We next investigated TLR3, which plays a critical role in antiviral defense. TLR3 recognizes viral dsRNA and is the most abundant TLR expressed in the placenta. 293T cells transfected with TLR3-specific siRNA significantly reduced TLR3 expression compared with control siRNA (FIG. 4E) but showed no change in the expression of 759-SAM-induced IFNL2/3 and IFNA2 (FIG. 4E), despite similar levels of C19MC activation, as assessed by RT-qPCR for miR-517a (FIG. 4E). Similarly, TLR3-siRNA transfected DICER-KO cells exhibited significant reduction in TLR3 expression (FIG. 4F) but showed no reduction in 759-SAM-induced IFNL2/3 and IFNA2 expression (FIG. 4F).

Lastly, we investigated the role of dsRNA-dependent protein kinase R (PKR/EIF2AK2) in IFN induction by C19MC. 293T cells transfected with PKR-specific siRNA significantly reduced PKR expression, as assessed by immunoblotting (FIG. 4G) and RT-qPCR (FIG. 4H), compared with control siRNA. Although 293T cells transfected with si-PKR and 759-SAM significantly increased the expression of C19MC miRNAs, as assessed by RT-qPCR for miR-517a, to levels similar to those in 293T cells transfected with si-control and 759-SAM (FIG. 4H), the expression of IFNL2/3, but not IFNA2, was significantly reduced (FIG. 4H). Similarly, PKR-siRNA-transfected DICER-KO cells exhibited a significant reduction in PKR expression, as assessed by immunoblotting (FIG. 4I) and RT-qPCR (FIG. 4J), compared with control siRNA, and the expression of 759-SAM-induced IFNL2/3, but not IFNA2, was significantly reduced in si-PKR-transfected cells compared with control siRNA (FIG. 4J). Taken together, these results reveal that the elevated induction of type III IFN by C19MC Alu dsRNA is mediated by RLR and PKR signaling pathways, whereas IFNA2, which is induced to a lesser extent by C19MC activation, is mediated by the RLR pathway.

### Example 6 Alu dsRNA Co-Localizes with C19MC miRNA in the Human Placenta

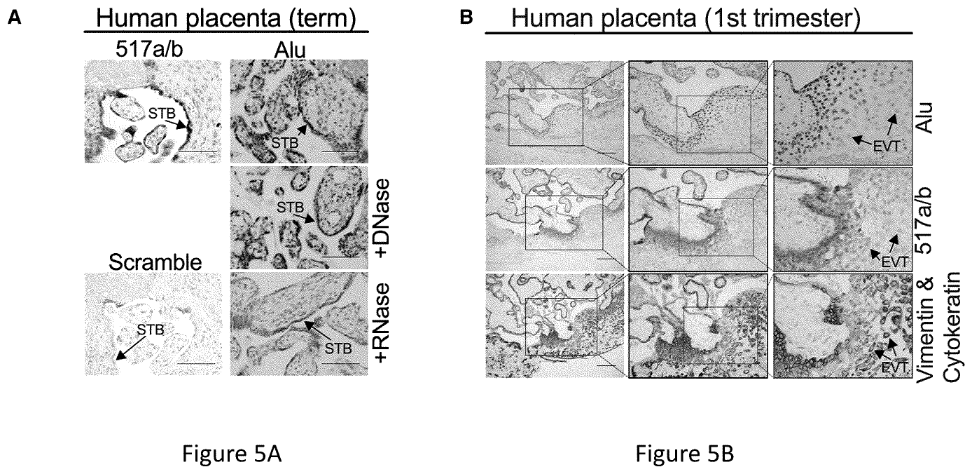

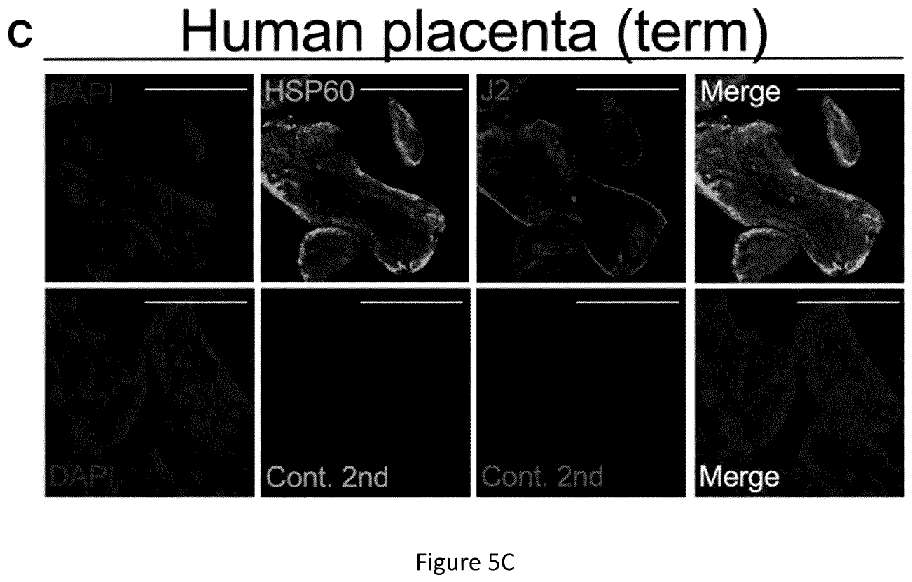

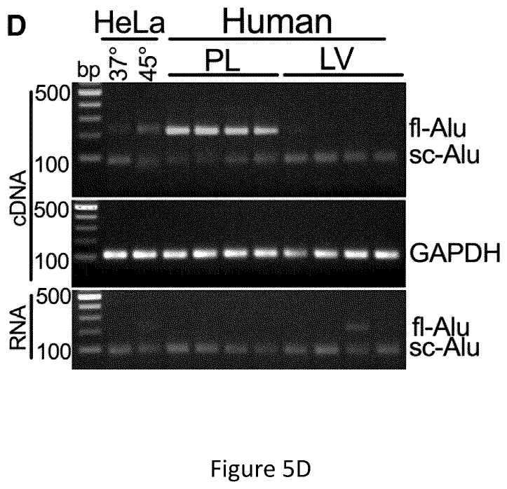

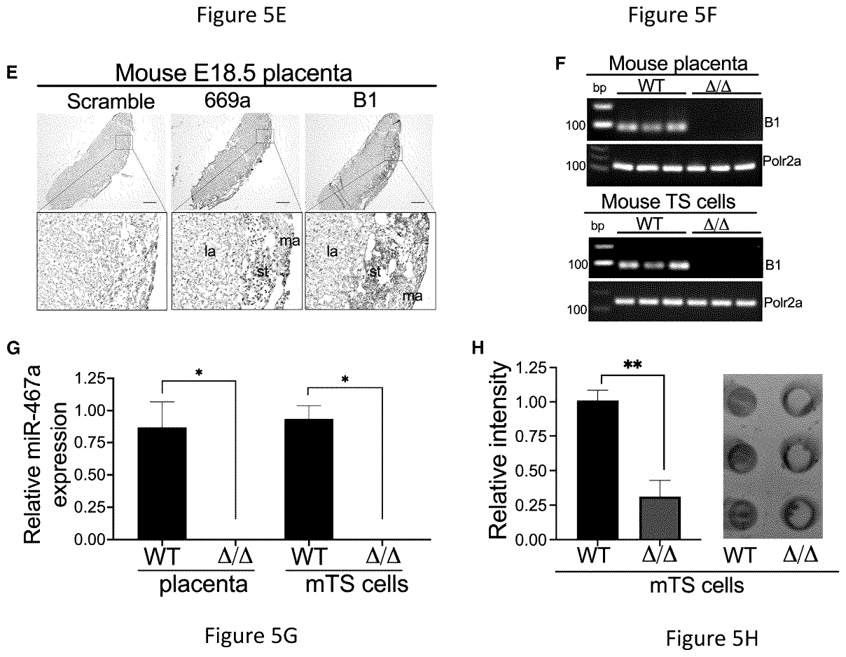

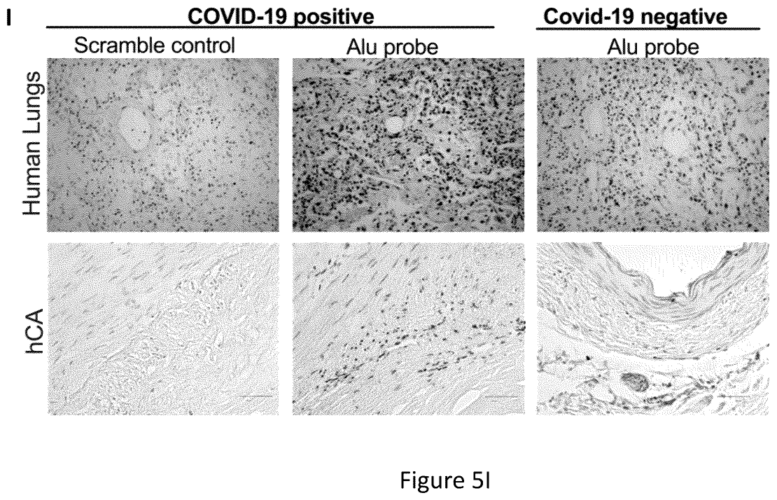

C19MC is highly expressed in the human placenta, particularly in the syncytiotrophoblast (STB) cell layer that covers the entire surface of the chorionic villi and becomes fully hemochorial by the end of the first trimester. The STB provides a robust physical and immunological barrier to limit vertical transmission. Previous reports have shown that mid-gestation human chorionic villous explants constitutively release type III IFN through an unknown mechanism and protect against ZIK V infection. 10 To examine in situ whether the Alu RNA co-localizes with the miRNAs of C19MC in the STB, we performed in situ hybridization of term human placental sections using probes designed to recognize miR-517a/b, Alu RNA transcripts, or a scrambled control. To confirm that the Alu probe recognizes Alu RNA and not Alu SINEs in the genomic DNA, we pre-treated the sections with either RNase A or DNase I, respectively. We observed that similar to the miR-517a/b, a strong Alu signal was observed in the STB layer, which was abolished when sections were pretreated with RNase A and not when sections were pre-treated with DNase I (FIG. 5A). We also used consecutive serial sections of first-trimester human placentas, which we have previously shown to robustly express miR-517a/b in the villous cytotrophoblast (CTB) with a gradual decrease as CTBs differentiate from proximal (proliferative) to distal (invasive) EVTs in the anchoring villi.21 To distinguish trophoblast (fetal) from decidual cells (maternal), we also performed immunohistochemical staining for cytokeratin and vimentin, respectively. We found that, similar tomiR-517a/b, Alu RNAs were also highly expressed in the villous trophoblast and proximal trophoblastic cell columns in anchoring villi and gradually decreased as the trophoblasts differentiated and invaded the decidua (FIG. 5B). To confirm that the Alu RNA that are highly expressed in the CTBs and the STB layer form dsRNA, we performed immunostaining using the J2 dsRNA-specific antibody of term human placental sections. To exclude the reactivity of the J2 mAb with mitochondrial dsRNA,27 we performed co-immunostaining of J2 and mitochondrial marker 60 kDa heat shock protein (HSP60) antibodies. Similar to miR-517a/b and Alu RNA, we found a strong and distinct dsRNA signal in the STB cell layer that did not colocalize with the mitochondrial marker (FIG. 5C). These results confirm that in the STB cell layer, the high expression levels of C19MC coincide with the high levels of Alu RNA and dsRNA.

The increase in Alu expression in the human placenta were also verified by competitive RT-PCR for Alu using RNA isolated from human term placentas, and human cardiac left ventricle samples were used and HeLa cells after heat shock recovery as control, as well as PCR on total RNA to exclude the amplification of genomic Alu SINEs. Results showed that fl-Alu expression was highly increased in the placentas compared with the left ventricles, whereas control PCR on total RNA showed no products in the placental samples (FIG. 5D). The fl-Alu RT-PCR products of two placentas were cloned and sequenced (n=20 and n=19), and all aligned with Alu SINEs (Table 5). Together, our results show that Alu RNA are highly expressed and colocalize with the C19MC miRNAs in the STB and CTB cell columns of the human placenta.

Viral infections including SARS-COV-2 were reported to induce Alu RNA expression. To test our methods in additional tissues, we performed in-situ hybridization of COVID-19 positive lungs and human coronary artery sections using the sane LNA Alu probe or a scrambled control as previously used in placental tissue. We found that indeed Alu RNA are highly expressed in the COVID-19 positive lungs and coronary artery compared to uninfected donors (FIG. 5I). Thus, demonstrating that our methods can be used in multiple tissues.

### Example 7 B1 RNA Co-Localizes with C2MC miRNA in the Mouse Placenta

To further investigate the role of SINE RNA in the placental IFNL response in vivo, we turned to a mouse model because the C2MC, similar to the primate C19MC, is also rich in rodent-specific B1 SINEs and is robustly expressed in the placenta. We first determined whether C2MC miRNA and B1 RNA also colocalize in the mouse placenta. We performed in situ hybridization of E18.5 WT placentas using probes specific to C2MC-member miR-669a-3p, B1 RNA, or a scrambled control and found that both miR-669a-3p and B1 RNA are highly expressed in the spongiotrophoblast cell layer (FIG. 5E). To confirm the co-expression of C2MC miRNAs and B1 RNA, we used placentas and trophoblast stem (mouse trophoblast stem [mTS]) cells derived from WT or C2MC KO (C2MC^{Δ/Δ}) mice that lack intron 10 of the Sfmbt2 gene. 17 We performed RT-PCR using primers that recognize the C2MC B1 consensus sequence and RT-qPCR for a representative C2MC miRNA, miR-467a. We found that B1 (FIG. 5F) and miR-467a (FIG. 5G) were highly expressed only in WT placentas and mTS cells, demonstrating that, indeed, C2MC expresses B1 RNA together with the C2MC miRNAs. Next, we tested whether C2MC generates dsRNA and found that compared with WT, mTS cells derived from the C2MC^{Δ/Δ} mice showed a significant decrease in dsRNA (FIG. 5H).

### Example 9 C2MC Induces Constitutive IFN Response and Protects Against Vertical Viral Transmission

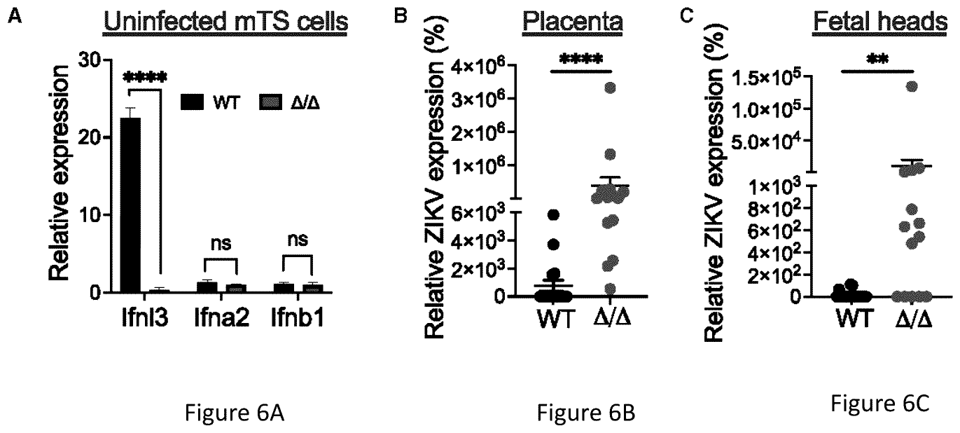

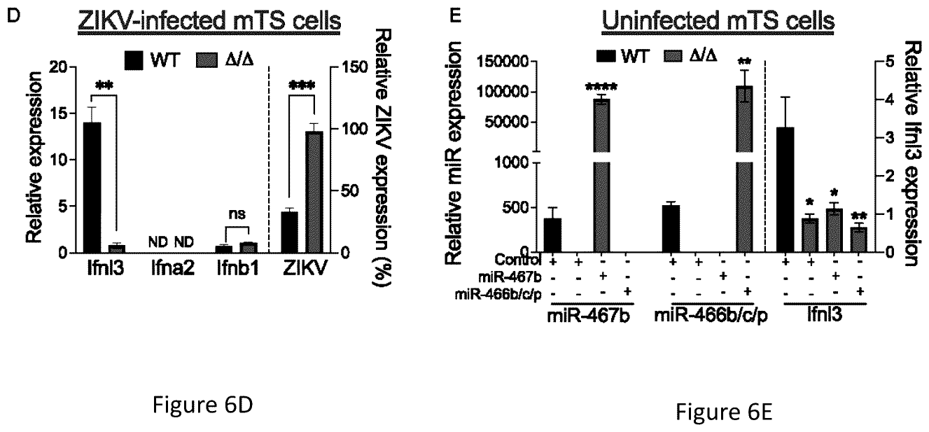

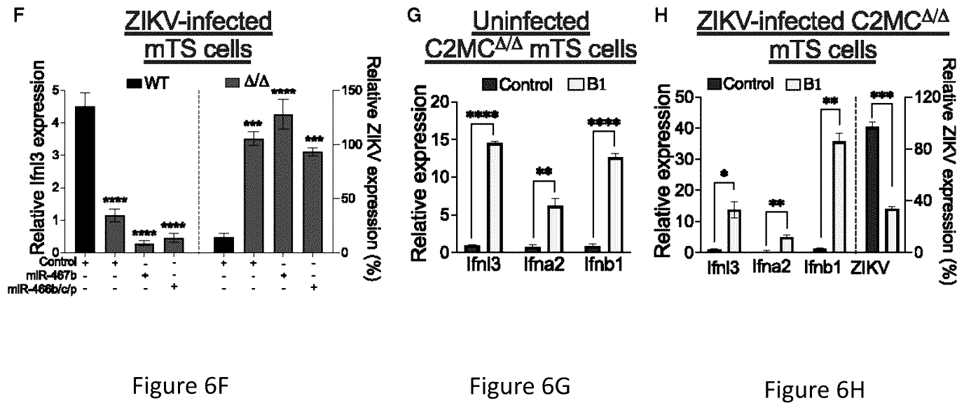

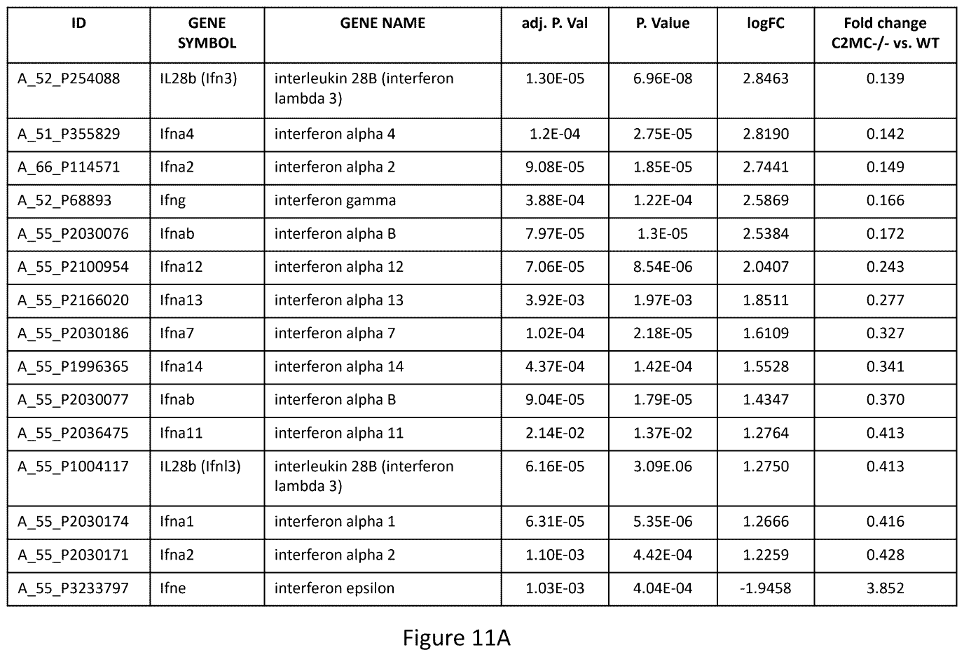

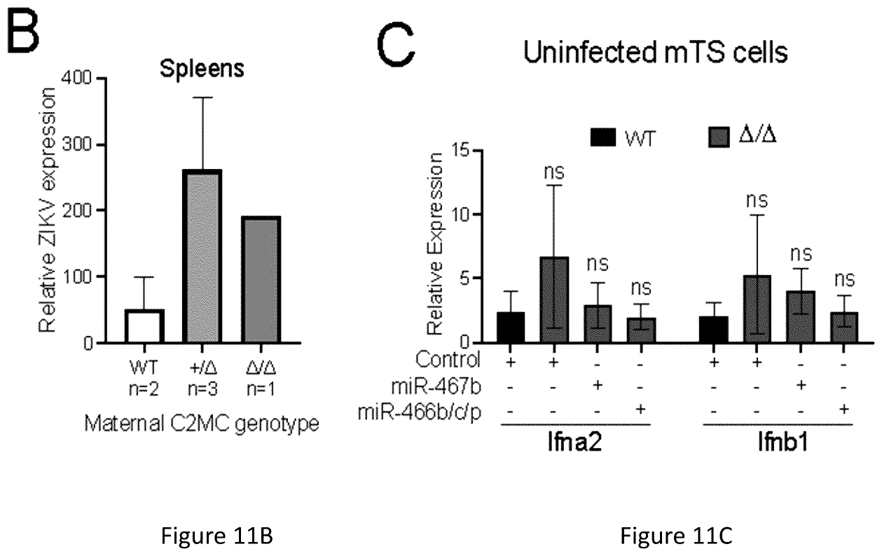

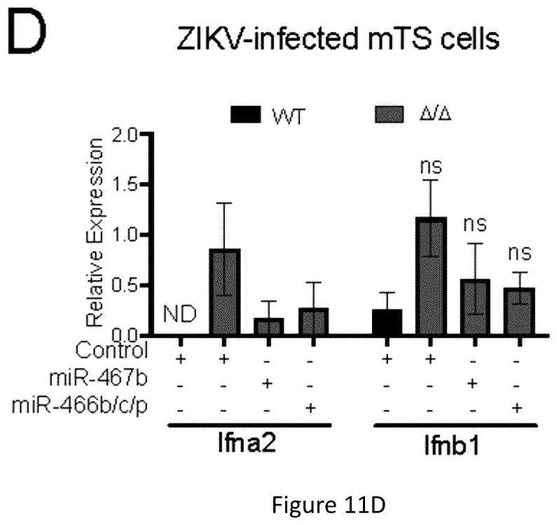

To test whether C2MC expression induces IFN expression in the mouse placenta and in the mTS cells, we analyzed previously published microarray data17 of WT and C2MC^{Δ/Δ} E11.5 mouse placentas (GEO: GSE82055) using the GEO2R platform and performed RT-qPCR for Ifnl3, Ifna2, and Ifnb1. We found a significant decrease in the expression of all three types of IFNs in C2MC^{Δ/Δ} placentas compared with the WT (FIG. 11A); however, C2MC^{Δ/Δ} mTS cells showed a significant decrease only in Ifnl3 expression, whereas Ifna2 and Ifnb1 expression showed no significant difference (FIG. 6A). To investigate the role of C2MC-induced IFN in the protection against viral vertical transmission in vivo, we intraperitoneally injected pregnant dams with ZIKV on E9.5 after pre-treatment with a type I-IFN-receptor-blocking antibody. After 5 days, ZIKV replication in maternal spleens, placentas, and fetal heads was measured by RT-qPCR. Although dams showed no significant difference in ZIKV expression in their spleens (FIG. 11B), the placentas (FIG. 6B) and fetal heads (FIG. 6C) of the C2MC^{Δ/Δ} pups exhibited significantly higher levels of ZIKV compared with WT. To further establish the role of C2MC in the antiviral protection, we infected mTS cells derived from WT and C2MC^{Δ/Δ} mice with ZIKV. We observed that even after ZIKV infection, C2MC^{Δ/Δ} mTS cells exhibited persistently lower levels of Ifnl3 and significantly increased viral replication compared with WT mTS cells, whereas Ifna2 and Ifnb1 expressions were not changed (FIG. 6D). These results strongly suggest that C2MC mediates the constitutive IFN expression in the mouse placenta and provides intrinsic protection against vertical viral transmission.

### Example 9 C2MC B1 RNA and not the miRNA Induces Constitutive IFN Expression and Antiviral Protection

To determine whether the C2MCmiRNA or the B1RNA is responsible for the constitutive IFN expression, we transfected C2MC^{Δ/Δ} mTS cells with miR-467b or miR-466b/c/p mimics that represent the two major C2MC miRNA families. Despite the increase in the expression levels of miR-467b or miR-466b/c/p in C2MC^{Δ/Δ}mTS cells to >100-fold higher than those in WT mTS cells transfected with control mimic (FIG. 6E), the expressions of Ifnl3 (FIG. 6E), Ifna2, and Ifnb1 (FIG. 11C) were not increased. Moreover, C2MC^{Δ/Δ}mTS cells transfected with miR-467b or miR-466b/c/p mimics remained highly susceptible to ZIKV infection (FIGS. 6F and 11D). These results indicate that C2MC-induced IFN and antiviral response is independent of the miRNAs. To evaluate the role of C2MC B1 RNA in this response and because C2MC B1 SINEs are all arranged in the forward orientation on the positive strand, we transfected C2MC^{Δ/Δ} mTS cells with 100%-pseudouridine-substituted (to reduce its immunogenicity 28) IVT C2MC B1 RNA in the forward direction. We found that compared with control 100%-pseudouridine-substituted IVT GFP mRNA, B1 RNA significantly induced Ifnl3, Ifna2, and Ifnb1 expressions in C2MC^{Δ/Δ} mTS cells (FIG. 6G) and inhibited ZIKV replication (FIG. 6H). Taken together, our results establish the role of C2MC B1 RNA, and not the miRNAs, in eliciting constitutive IFN expression and antiviral protection in the mouse placenta.

### Example 10 miRNA Clusters Regulate the Expression of Immune Tolerance Genes

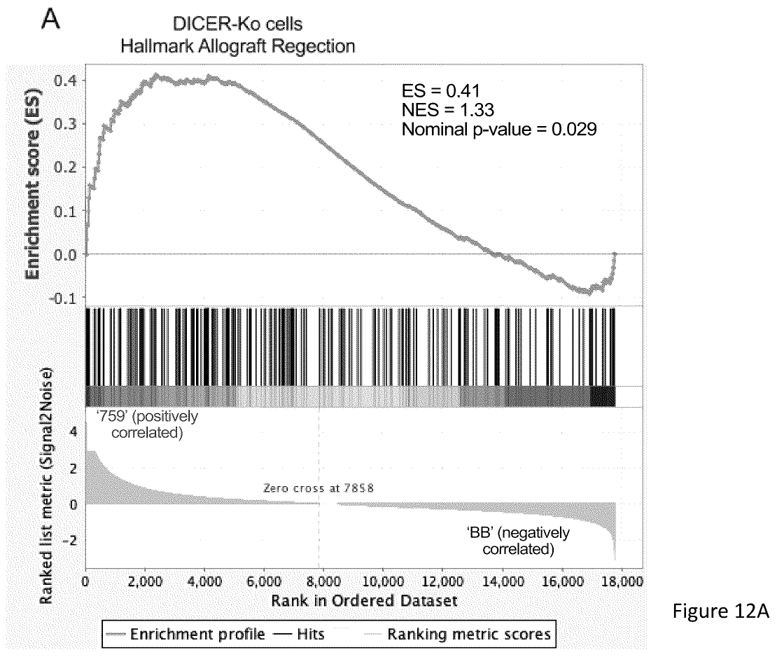

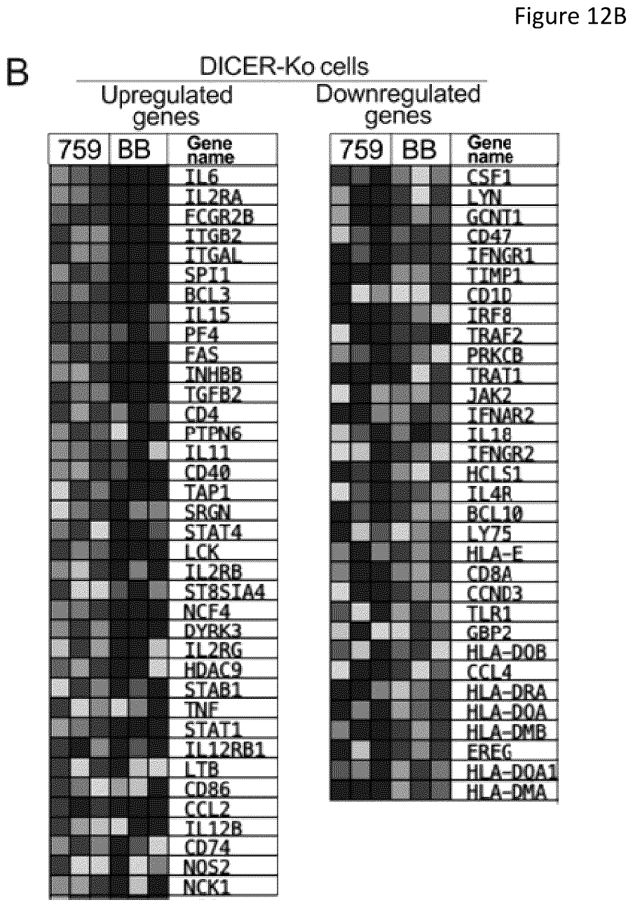

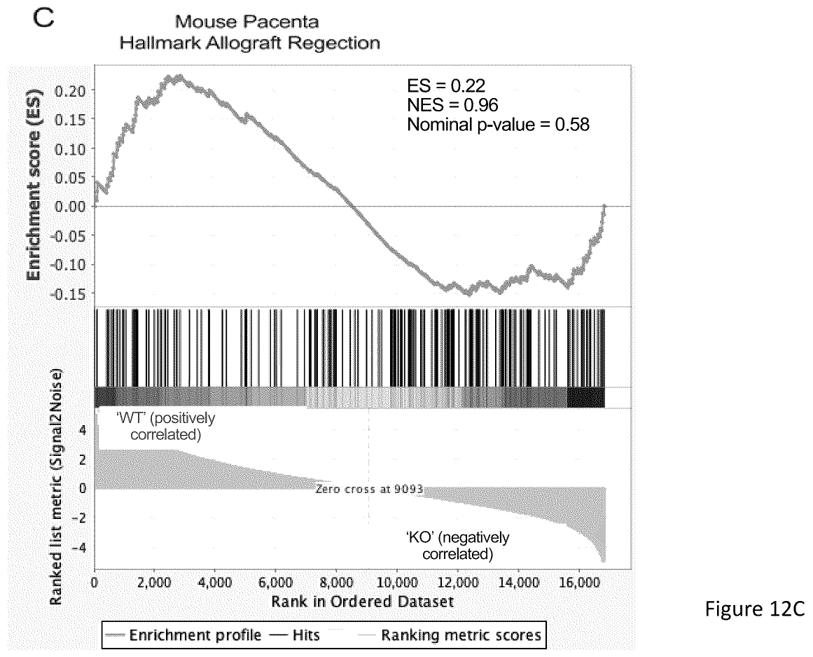

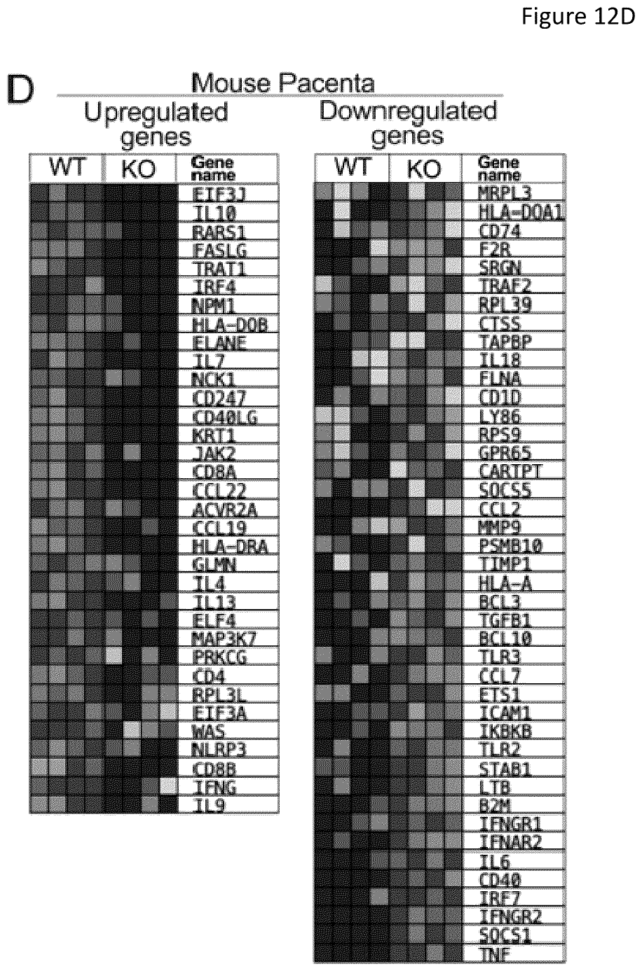

To investigate the regulatory role of C19MC in immunotolerance, particularly considering its Alu-mediated involvement in the IFNL response and protection against viral infections, we conducted hallmark gene set enrichment analysis (GSEA) of DICER-KO cells transfected with 759-SAM, compared to control BB-SAM transfected cells. Our investigation revealed a significant enrichment of hallmark of allograft rejection (FIG. 12A). Specifically, we observed an upregulation of several cytokines including IL11¹⁷ and TGFB¹⁸, which play a crucial role in maternal immunotolerance towards the semi allograft fetus (FIG. 12B). Conversely, our findings indicated a downregulation of several major histocompatibility complex genes as well as proinflammatory cytokines, such as IL18, associated with allograft rejection and recurrent pregnancy loss¹⁹ (FIG. 12B).

Remarkably, the gene expression profiles of E11.5 placenta derived from C2MC^{Δ/Δ}, when compared to those from C2MC^{+/+} mice, demonstrated enrichment in the hallmark of allograft rejection (FIG. 12C). Specifically, there was a notable downregulation in numerous cytokines associated with immunotolerance including IL10²⁰ and IL9²¹ (FIG. 12D). Concurrently, there was an upregulation of proinflammatory cytokines such as IL18 and major histocompatibility complex genes (FIG. 12D). These intriguing findings shed light on the intricate regulatory network orchestrated by the C19MC Alu RNA and the C2MC, providing insights into their potential role not only in mediating IFNL and antiviral protection but also in regulating immunotolerance, which warrants deeper exploration.

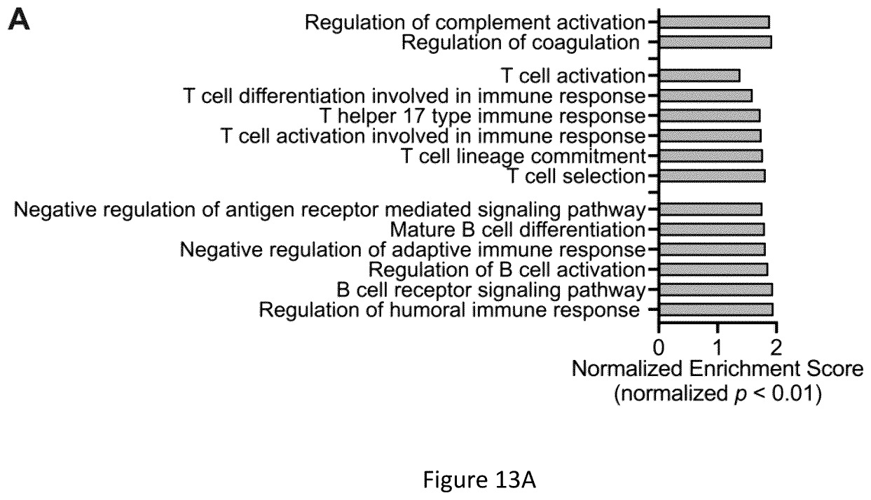

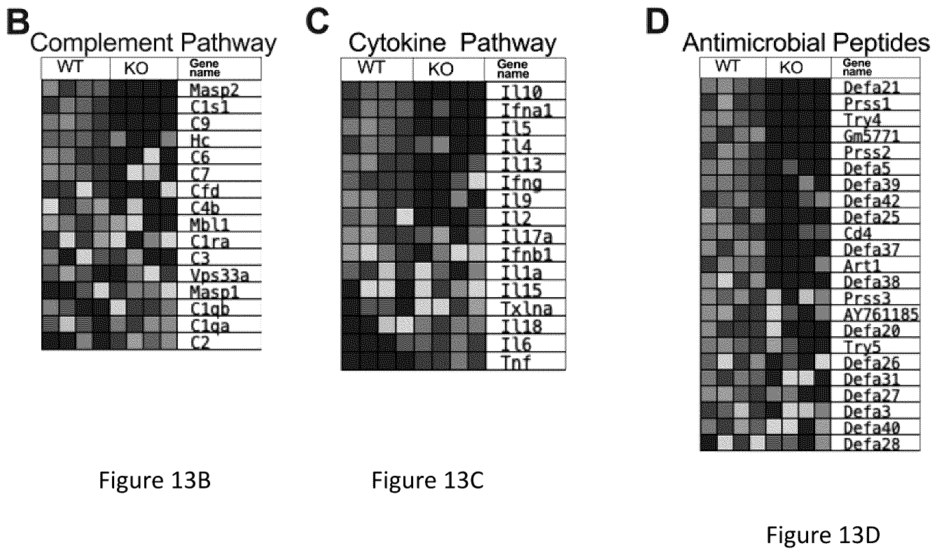

To investigate the regulatory role of C19MC Alu-mediated innate and adaptive immunity, beyond the IFNL response and its antiviral protection, we conducted GSEA of DICER-KO cells transfected with 759-SAM, comparing them to control BB-SAM transfected cells. Our analysis uncovered a significant enrichment of the regulation of complement activation and coagulation as well as adaptive humoral immune response, involving both B and T cell activation (FIG. 13A). Notably, the Th17 type immune response emerged as a prominently enriched pathway. Specifically, the expression of IL17F was significantly increased (21-fold, p=0.0285) in DICER-KO cells transfected with 759-SAM, compared to control BB-SAM transfected cells. Conversely, gene expression profiles of E11.5 placenta derived from C2MC^{Δ/Δ}, in comparison to those from C2MC^{+/-} mice, exhibited a significant reduction in IL17F (10-fold, p=1.72×10⁻⁸) and IL17C (5.2-fold, p=5.59×10⁻⁶). Moreover, GSEA revealed significant reduction in the complement (FIG. 13B) and anti-inflammatory cytokine pathways (FIG. 13C), as well as numerous AMPs (FIG. 13D).

Given that C2MC and C19MC are expressed in placental trophoblasts, which are fetal cells, these compelling findings illuminate the intricate regulatory network orchestrated by C19MC Alu RNA and C2MC. This regulatory influence extends beyond the realm of IFNL, offering valuable insights into their potential roles in shaping the innate and adaptive immune responses specifically within the fetal domain at the maternal-fetal interface. These revelations emphasize the need for thorough exploration to unravel the full extent of their impact on maternal-fetal immunotolerance during pregnancy.

### DISCUSSION

Understanding the mechanisms that provide fetal protection against vertical viral transmission is of paramount importance, considering the devastating teratogenic effects of certain viral pathogens. Primates and rodents possess invasive hemochorial placentas, which enhance the risk of vertical viral transmission, thus posing selection pressure to evolve robust antiviral mechanisms for fetal protection.

This disclosure fills the gap in the knowledge of the mechanism that drives the intrinsic expression of IFNL in human placental trophoblast. C19MC Alu dsRNA, which is highly expressed in the STB and CTB cells of the placenta, is demonstrated to constitutively induces type III IFNs mediated by RLR and PKR and protects against viral infection. mTS cells and placentas derived from C2MC^{Δ/Δ} mice are also shown to lose their intrinsic IFN expression and increase ZIKV infection and vertical transmission. These results support previous reports that showed that human trophoblast constitutively secretes type III IFNs,^{9-11,29} and may explain why many viruses that infect pregnant women, including SARS-COV-2, exhibit a low incidence of fetal infection.³⁰ In addition, these results provide insights into the possible role of SINEs in mediating the constitutive expression of ISGs and antiviral resistance of stem cells.³¹

SINEs constitute ~13% of the human genome.³² Although they evolved to play a role in gene regulation, their effects are considered mostly deleterious to the host, as their retrotransposition can give rise to mutagenesis either in somatic cells or in the germ line, establishing an allele frequency causing many diseases.³³ To protect genomic integrity from deleterious insertions of Alu SINEs, healthy somatic cells acquired multiple mechanisms to strictly regulate their expression.³⁴ However, under stress conditions, such as viral infections, they regain transcriptional activation by RNA Pol III and become the largest class of virus-inducible noncoding RNA.^{35,36} Unlike stress-induced RNA-Pol-III-transcribed Alu SINEs, which produce Alu RNA only in the forward orientation, 80% of the RNA-Pol-II-transcribed C19MC Alu RNAs are produced in the reverse orientation. To date, the roles of C19MC Alu RNA and virus-induced Alu RNA in the host antiviral protection have not been explored. These results provide evidence that in the human placenta, the constitutive expression of C19MC Alu RNA plays a major role in the constitutive IFNL induction and antiviral protection. Importantly, the antisense bias of the C19MC Alu SINEs provides added benefits, especially during viral infections, when RNA Pol III induces Alu RNA in the forward orientation, resulting in further increase in the formation of Alu dsRNA that enhances the production of IFN and antiviral protection. Thus, our results suggest that C19MC Alu RNA is an integral part of the placental innate immunity.

C19MC encodes for 46 precursor miRNAs and >350 Alu SINEs. Here, 759-SAM-transfected AD-293 cells are shown to increase the expression of C19MC miRNAs by 103-fold. In comparison, in human placenta, the expression of the same C19MC miRNAs were 1,000-fold higher. In fact, 35% of the total miRNAs in the human placenta belong to the C19MC.15 Each time C19MC is transcribed, in addition to the 58 mature miRNAs, it produces >350 Alu RNAs, which is 6 times more than the number of mature miRNAs. Indeed, the competitive Alu RT-PCR, in situ hybridization, and immunostaining for dsRNA show that term human placenta expresses high levels of Alu RNA and dsRNA in the STB cell layer. These results are in agreement with previous reports that show that primary human trophoblast constitutively expresses type III IFN and is resistant to viral infections.^{9,10} Intriguingly, in situ hybridization of first-trimester human placenta shows that as CTBs differentiate into EVT, they lose C19MC miRNA and Alu RNA expression. These results may explain why EVTs are more susceptible to viral infections compared with CTB and STB.37 Although Alu SINEs are considered mostly deleterious to the host, the transient nature of the placenta has enabled the high expression levels of the C19MC Alu dsRNA to mediate antiviral protection while ensuring that the deleterious long-term effects are negligible.

Unlike type I IFN, type III IFN protects the fetus from viral infection without posing high risk of pregnancy complications. 12 Here, we show that C19MC Alu dsRNA preferentially induces type III rather than type I IFNs through the cytosolic dsRNA sensing RLR and PKR, but not through the endosomal TLR3. Because the Alu-rich C19MC is transcribed by RNA Pol II in the nucleus and processed by DROSHA and DICER1 in the nucleus and cytoplasm, it is not surprising that the C19MC Alu dsRNA mediates IFNL induction through the cytoplasmic dsRNA sensors and not the transmembrane TLR3. Because C19MC and type III IFNs are located on the proximal long arm of chromosome 19, it is expected that in addition to the Alu dsRNA that mediates the RLR and PKR signaling transduction, C19MC Alu RNA may also serve as an enhancer RNA that functions in cis in a cell type-specific fashion to further enhance the transcription of type III IFNs. In contrast, type I IFNs, which are located on chromosome 9, are activated solely by the Alu-dsRNA-mediated RLR signaling transduction and not through the cis-acting enhancer Alu RNA. In contrast, C2MC and IFNs are located on different chromosomes in mouse, which may explain why in the mouse placenta, C2MC induces all types of IFN. These speculations warrant further investigations.

The evolution of the imprinted C19MC and C2MC coincided with complex placentation.¹⁴ These results provide evidence of the convergent evolution of hemochorial placental antiviral immunity mediated by lineage-specific SINEs that have clustered and coevolved with the maternally imprinted miRNA clusters after the primate-rodent split about 90 million years ago. Further studies are needed to investigate the potential roles of SINEs and other retrotransposons as well as other retrotransposon-rich coding or noncoding genes in the placental protection against diverse pathogens, including bacteria, fungi, and parasites.

In summary, the disclosed findings provide a paradigm shift in understanding of the host antiviral innate immunity and place SINEs as an integral component of this process, at the maternal-fetal interface and perhaps beyond, to ensure species survival.

### Materials and Methods

### Data and Code Availability

RNAseq and small RNAseq data have been deposited at Sequence Reads Archive (SRA) (www.ncbi.nlm.nih.gov/sra/) and are publicly available under the accession numbers SRA: PRJNA603843, PRJNA945507.

### Human Studies

Deidentified human term placentas from normal pregnancies were obtained with written, informed consent under the University of South Florida IRB Protocol 00015578. Previously banked first trimester placental paraffin specimens obtained from voluntary terminations of uncomplicated pregnancies were used after approval by the University of South Florida Institutional Review Board (Protocol 00019472). Written informed consent was received from patients prior to inclusion in the study. Human left ventricle samples were obtained from viable, non-transplantable deidentified human hearts from three female (age 56, 65 and 85 years) and one male (age 52 years) brain-dead donors after consent was obtained from next of kin in accordance with Florida State Statutes and The Declaration of Helsinki. Hearts were donated through the LifeLink Foundation. An MTA has been executed enabling transfer of non-transplantable organs for research at the University of South Florida.

### Animal Studies

C2MC^{Δ/Δ} mice were generated and kindly provided by the RIKEN BRC through the National Bio-Resource Project of the MEXT/AMED, Japan. 17 Breeding and experimental procedures were conducted with prior approval of the Animal Care and Use Committee at the University of South Florida under protocols 6183M and 9205R, respectively. WT (C57BL/6) and C2MC^{Δ/Δ} mice were housed under standard conditions. Dams between approximately 4-7 months of age and 19-25 g weight were used for mating and the detection of the vaginal mucus plug was taken to be indicative of E0.5. WT and C2MC^{Δ/Δ} male and female fetuses were used in the study. For in situ hybridization E18.5 WT placentas were used. For B1 RT-PCR and C2MC miRNA RT-qPCR E11.5 WT and C2MC^{Δ/Δ} placentas were used, whereas for in vivo ZIKV experiments E14.5 WT and C2MC^{Δ/Δ} placentas and fetal heads were used.

### Cell Culture

AD-293 cells (Stratagene Cat #240085), 293T cells (ATCC CRL-3216), DICER-Ko derived from 293T cells (2-20 cells provided by Dr. Bryan Cullen, Duke University),38 DROSHA-Ko derived from 293T cells (provided by Dr. David A. Williams, Harvard Medical School),39 MAVS-Ko derived from 293T cells (provided by Dr. Young Bong Choi, Johns Hopkins University School of Medicine) 40 and Vero-E6-high ACE2 (BEI Resources #NR-53726) cells were grown in DMEM (Genesee Scientific #25500) supplemented with 10% heat-inactivated fetal bovine serum (FBS; Millipore-Sigma #F4135). HTR8/SVneo (ATCC CRL-3271) cells were cultured in RPMI 1640 (Gibco #11875) supplemented with 10% heat-inactivated FBS. Vero cells (ATCC CCL81, a gift from Dr. Bala Chandran, University of South Florida) were grown in DMEM/F-12, HEPES, no phenol red (Gibco, #11039021) with 10% non-heat inactivated FBS (Corning #35-010-CV). HEp-2 cells (ATCC CCL-23; a gift from Dr. Subra Mohapatra, University of South Florida) and HeLa cells (ATCC CCL-2) were grown in minimum essential medium (Gibco #11095080) and Eagle's minimum essential medium (ATCC 30-2003), respectively, supplemented with 10% heat-inactivated FBS. All cell culture media was supplemented with 1% Pen Strep (Gibco #15070063) and 1% L-glutamine (Gibco #25030081). Mouse trophoblast stem (mTS) cells were provided by the RIKEN BRC through the National BioResource Project of the MEXT, Japan and grown as previously described with minor modifications.53 Briefly, mTS cells were cultured in plates coated with 15 mg/mL human plasma fibronectin (EMD Millipore FC010) for 2 hours in CDM/FAXY media, generated by combining 1:1 Neurobasal medium (Gibco #21103049): DMEM/F-12 (Ham's) (Gibco #11320033) supplemented with 1% KnockOut serum (Gibco #10828028), 1% N-2 supplement (Gibco #17502048), 1% B-27 supplement (Gibco #17504044), 1% Pen Strep (Gibco #15070063), 1% GlutaMax (Gibco #35050061), 0.05% bovine serum albumin (EMD Millipore #126575), 150 mM 2-mercaptoethanol (Gibco #21985023), 50 ng/ml recombinant murine FGF-basic (PeproTech #450-33), 20 ng/ml recombinant human/murine/rat activin A (PeproTech #120-14E), 10 mM XAV939 (PeproTech #2848932) and 5 mM Y-27632 dihydrochoride (PeproTech #1293823). Medium was replaced every 2 days and to dissociate cells between passages TrypLE Express (Gibco #12604013) was used. 293T-ACE2 cells were generated by transducing 293T cells with pLenti-hACE2-hygro expressing lentivirus, a gift from Dr. Neville Sanjana (Addgene plasmid #161758; http://n2t.net/addgene: 161758; RRID: Addgene_161758),45 followed by selection with hygromycin (InvivoGen #ant-hg-1). All cell lines were cultured at 370C and 5% CO2. Cell line sex determination and authentication were not performed. However, the cell genotypes were confirmed by RT-PCR, RT-qPCR and Western blotting and were all tested routinely with the Universal Mycoplasma Detection Kit (ATCC 30-1012K) to ensure that they were free of Mycoplasma contamination.

### Cell Transfections

For C19MC transcriptional activation cells were transfected as previously described.21 Briefly, cells were transfected with a 1:1:1 mass ratio of dCAS9-VP64-GFP (Addgene plasmid #61422; http://n2t.net/addgene: 61422; RRID: Addgene_61422), MS2-P65-HSF1-Hygro (Addgene plasmid #61426; http://n2t.net/addgene: 61426; RRID: Addgene_61426) and the lenti sgRNA (MS2)_zeo backbone plasmid (Addgene plasmid #61427; http://n2t.net/addgene: 61427; RRID: Addgene_61427) a gift from Feng Zhang.44 C19MC specific sgRNAs #759, #620 and the CYP19A1 specific sgRNA #125.3 (Table S6, oligos #1-3) were cloned into the lenti sgRNA (MS2)_zeo backbone plasmid. AD-293, 293T, DICER-Ko, DROSHA-Ko, MAVS-Ko cells were transfected using Lipofectamine 2000 (Invitrogen #11-668-019) HTR8/SVneo cells using Lipofectamine 3000 (Invitrogen #3000015). The culture medium was replaced after 24 hours, and the transfected cells were incubated for a total of 60-72 hours.

For pattern recognition receptor knock down experiments cells were transfected with MAVS siRNA (h) (Santa Cruz Biotechnology, Inc; sc-75755), PKR siRNA (h) (Santa Cruz Biotechnology, Inc; sc-36263), or Control siRNA-A (Santa Cruz Biotechnology, Inc; sc-37007). For TLR3 (human) Silencer Select Validated siRNA (Life Technologies Corporation, s235; Cat #4427038), Silencer Select Negative Control siRNA #2 (Life Technologies Corporation; Cat #4390846) was used. After 24 hours, cells were transfected with 759-SAM or BB-SAM for 48 hours.

For experiments that studied the autocrine/paracrine effects of IFNL3, cells were transfected with 759-SAM, BB-SAM or GFP. After six hours, the media was replaced with or without 400 ng/ml of anti-human IFNL3 antibody (R&D Systems Cat #DY1598B-05) and collected after 66 hours.

mTS cells were transfected with miR-467b-5p (Life Technologies #MC11605), miR-466b/c/p-3p (Life Technologies #MC19359) mimic, miRNA mimic negative control (ThermoFisher Scientific #4464058), IVT BI SINE RNA or control GFP-mRNA using Lipofectamine 3000 (Invitrogen #3000015) according to the manufacturer's instructions for 24 hours.

AD-293 and mTS cells were transfected with 10 mg/mL of Poly(I:C) HMW (InvivoGen #31852-29-6) for 24 hours and collected for RNA extraction to be used as positive controls in RT-PCR and RT-qPCR experiments.

### In Vitro Transcription

To generate Alu RNA transcripts, the bacterial artificial chromosome containing C19MC (BACPAC Resources, RP11-1055017) was used as a template with the previously described 5′-exon (2) and 3′-exone (2) primers20 (Table S6, oligos #4) to obtain a PCR product containing fragments of C19MC. PCR products were cloned into the pCR_4Blunt-TOPO_vector by one-step cloning using the Zero Blunt_TOPO_PCR Cloning Kit (Invitrogen #45-0031). Following transformation into GC competent cells (Genesee Scientific #42-661), single colonies were picked and sequenced. A colony that contained a fragment of C19MC that aligned to chr19:53,678,369-53,681,590 on the GRCh38/hg38 human genome was used as the template for PCR amplification. For IVT AluJb, AluSx, and AluSz in the forward direction, we included the T7 promoter sequence in the forward primer (Table S6, oligos #5-7), and to IVT the Alus in the reverse strand, we added the T7 promoter sequence to the reverse primer (Table S6, oligos #8-10).

To generate C2MC B1 RNA in the forward direction, C2MC B1 consensus sequence was PCR amplified using forward and reverse primers (Table S6, oligos #11) and cDNA of WT mTS cells that was generated using a mixture of random primer hexamers and anchored-dT primer (New England Biolabs #S1330S) and M-MuLV reverse transcriptase (New England Biolabs #M0253L). The B1 PCR product was cloned into the pCR_4Blunt-TOPO_vector by one-step cloning using the Zero Blunt TOPO_PCR Cloning Kit (Invitrogen Cat #45-0031). Following transformation, single colonies were picked and sequenced. A colony that contained the C2MC B1 in the forward direction was used as the template for PCR amplification using the same primers (Table S6, oligos #11) but the forward primer contained T7 promoter (Table S6, oligos #12). The resulting PCR product was used as a template for IVT.

Control GFP mRNA was IVT as previously described.54,55 Briefly, human b-globin 30 UTR (132 bp) was amplified using HeLa genomic DNA, extracted using the Monarch Genomic DNA Purification Kit (New England Biolabs #T3010S) as a template, with forward and reverse primer that include EcoRI restriction site (Table S6, oligos #13). PCR product was EcoRI digested and cloned into the pLL3.7 plasmid a gift from Luk Parijs46 (Addgene plasmid #11795; http://n2t.net/addgene: 11795; RRID: Addgene_11795). Template for GFP IVT were generated by PCR using forward primer containing a T7 promoter, 50 UTR of human b-globin and the first 26 bases of GFP and the same reverse primer of the human b-globin 30 UTR (Table S6, oligos #14).

The HiScribe T7 High Yield RNA synthesis kit (New England Biolabs, E2040S) was used for IVT according to the manufacturer's instructions with 100% substitution of uridine with pseudouridine (TriLink Biotechnologies, N-1019) to reduce the immunogenicity of the IVT mRNA.28 After ammonium acetate precipitation, IVT RNA was washed with 70% ethanol, resuspended in H2O, quantified by spectrophotometry, and stored at −80_C until use.

The GFP RNA was then capped using the ScriptCap m7G Capping System (CellScript, C-SCCE0625) and poly-A tailing using the A-Plus Poly(A) Polymerase Tailing Kit (CellScript, C-PAP5104H) prior to ammonium acetate precipitation.

### HeLa Heat Shock

Heat-shock-induced Alu expression was performed as previously described.26 Briefly, HeLa cells were heat-shocked at 45° C. for 30 minutes and then allowed to recover for 4 hours in standard culture conditions at 37° C. before total RNA extraction.

### Protein Extractions and Western Blotting

Cells were lysed as previously described56 in ice-cold lysis buffer containing 40 mM HEPES [pH 7.5], 120 mM NaCl, 1 mM EDTA, 10 mM pyrophosphate, 10 mM glycerophosphate, 50 mM NaF, 0.5 mM orthovanadate, EDTA-free protease inhibitors [Roche] and 0.3% CHAPS for 20 minutes. After clearing the lysates by centrifugation at 13,000 3 g for 10 min at 4° C., samples were stored at −80° C.

Protein lysates were fractionated by SDS-PAGE and transferred to BioTrace NT Nitrocellulose transfer membranes (Pall Life Sciences Cat #27376-991). Membranes were incubated overnight at 4_C with rabbit anti alpha tubulin (Cell Signaling Technology Cat #2144, RRID:AB_2210548, 1:1000), rabbit anti-DICER1 (D38E7) (Cell Signaling Technology Cat #5362S, RRID:AB_10692484, 1:1000), rabbit anti-DROSHA (D28B1) (Cell Signaling Technology Cat #3364, RRID:AB_2238644, 1:1000), rabbit anti-IRF7 (Cell Signaling Technology Cat #4920, RRID:AB_2127551, 1:1000), rabbit anti-human MAVS (Cell Signaling Technology Cat #3993; RRID:AB_823565, 1:1000), rabbit anti-human PKR (Cell Signaling Technology Cat #3072S; RRID:AB_2277600, 1:1000) or rabbit anti-GAPDH (14C10) (Cell Signaling Technology Cat #2118, RRID:AB_561053, 1:2000) primary antibodies in Odyssey Blocking Buffer (PBS, LI-COR Biosciences, Fisher scientific 15590545). After washing three times in TBS-Tween, the membranes were incubated for 20-minutes at room temperature with IRDye 680 donkey anti-rabbit IgG secondary antibody (LI-COR Biosciences Cat #926-68073, RRID:AB_10954442, 0.2 mg/ml). Membranes were washed three times in TBS-Tween, imaged on Odyssey CLX-2050 imaging system (LI-COR instrument) and analyzed using Image Studio acquisition software (LI-COR, ver 5.2).

### RNA Extraction, RT-PCR and RT-qPCR

Cultured cells, term human placenta, mouse placentas, fetal heads and spleens were lysed using Qiazol lysis reagent (Qiagen) on ice. Total RNA was extracted using the miRNeasy Kit (Qiagen #217004) and treated with RNase-free DNase (Qiagen #79254) according to the manufacturer's instructions. RNA was quantified spectrophotometrically at 260 nm and stored at −80° C.

One mg total RNA was used for reverse transcription with a mixture of random primer hexamers and anchored-dT primer (New England Biolabs #S1330S) or oligo (dT) primers (New England Biolabs #S1316S) and M-MuLV reverse transcriptase (New England Biolabs #M0253L) according to the manufacturer's recommendations. For miRNA, 0.5 mg total RNA was reverse transcribed using the TaqMan miRNA Reverse Transcription Kit (Thermo Fisher Scientific #4366596) according to the manufacturer's instructions.

To assess relative mRNA and miRNA expression levels Applied Biosystems TaqMan Fast Universal PCR Master Mix (2×), no AmpErase_UNG (Fisher Scientific #4352042) and the TaqMan RT-qPCR probes were used according to the manufacturer's instructions and run on QuantStudio 3 Real-Time PCR system (Life Technologies). The following TaqMan RT-qPCR probes were used: IFNL2/3 (Hs04193048_gH), IFNB1 (Hs01077958_s1), ISG15 (Hs00192713_m1), OAS1 (Hs00973637_m1), APOBEC2 (Hs00 199012_m1), APOBEC3G (Hs00222415_m1), TNF (Hs01113624_g1), IL6 (Hs00174131_m1), IFITM1 (Hs01652522_g1), TLR3 (Hs00 152933_m1), EIF2AK2 (Hs00169345_m1), CYP19A1 (Hs00903411_m1), IFNA2 primers (forward 5′-CTTGAAGGACAGACATGACTTTGGA (SEQ ID NO: 80), Reverse 5′-GGATGGTTTCAGCCTTTTGGA (SEQ ID NO: 81) and FAM probe 5′-TTCCCCAGGAGGAGTTTGGCAACC) (SEQ ID NO: 82),41 GAPDH (Hs027 86624_g1), hsa-miR-517a (002402), hsa-miR-515-5p (001112), hsa-miR-516b (001150), hsa-miR-518c (002401), hsa-miR-519d (002403), U18 (001204), mouse Ifnl3 (Mm00663660_g1), mouse Ifna2 (Mm00833961_s1), mouse Ifnb1 (Mm00439552_s1) mmumiR-467a (002587), mmu-miR-467* (001671), mmu-miR-466b/c/p (464896_mat), and mmu-snoRNA202 (001232). For VSV, ZIKV, RSV and SARS-COV-2, GAPDH, Polr2a RT-qPCR were performed using PowerUpSYBR Green Master Mix (Applied Biosystems #A25741) using the primer sets described in Table S6, oligos #15-20. For MAVS RT-qPCR the predesigned KiCqStart SYBR Green primers for human MAVS H_MAVS_2 (Millipore Sigma). Samples were run in triplicate and the relative expression was calculated using the 2-DDCt method. 57

RT-PCR for IFNL2/3, Alu, GAPDH, and Polr2a were performed using primers listed in Table S6, oligos #19-22, RT-PCR for multiple subtypes of human IFNA42 and IFNB43 were performed with previously described primers, and B1 RT-PCR was performed using oligos #11 with either Taq DNA Polymerase with ThermoPol buffer (New England Biolabs #M0267X) or PowerUpSYBR Green Master Mix (Applied Biosystems #A25741). The PCR products were run on a 1.5% agarose gel. For flAlu:scAlu ratio quantifications, the band intensities of the PCR products were quantified with ImageJ software (version 1.53u) 52 and normalized to GAPDH after subtracting background intensity. To control for genomic DNA contamination, the Alu PCR reaction was also run on the total RNA of the same samples and visualized on an agarose gel. The Alu PCR products were gel purified and cloned into the pCRTM4-TOPO_TA vector and pCRTM2.1-TOPO_vector using the TOPO_TA Cloning™ Kit (Invitrogen Cat #45-0030). After transformation, plasmid DNA was extracted from single colonies and sent for sequencing using T3 and T7 primers or M13 forward and reverse primers. The sequencing results are shown in Table S5 after trimming the vector sequences using the flanking EcoRI restriction sites. The sequences were verified to be Alu SINEs using BLAT alignment against the human GRCh38/hg38 genome in the USCS Genome Browser.

Dot Blotting of dsRNA

One mg of total RNA in 10 mL were loaded onto a wet Biotrace Nitrocellulose membrane (Pall Biosciences) using a Minifold dot-blotter (Schleicher & Schuell, Inc.). The membrane was then baked at 80_C for 1 hour before blocking for 1 hour at room temperature in Odyssey Blocking Buffer (PBS, LI-COR Biosciences, Fisher scientific 15590545) and probed overnight at 4_C with J2 mouse monoclonal anti-dsRNA antibody (Scicons, 1:1000). After TBS-Tween washing the membranes were incubated with IRDye 800CW goat antimouse secondary antibody (LI-COR Biosciences Cat #926-32210, RRID:AB_621842, 0.2 mg/mL) at room temperature for 20 minutes. The resulting immunoblots were washed again with TBS-Tween, scanned and quantitatively assessed using the Odyssey CLX-2050 imaging system (LI-COR instrument) and analyzed using Image Studio acquisition software (LI-COR, ver 5.2).

### ELISA

Enzyme-linked immunosorbent assays for human IFNL1/3 (R&D Systems #DY1598B-05) and all subtypes of IFNA (R&D Systems #DFNASO) were performed according to the manufacturer's instructions on conditioned cell culture media collected 72 hours after transfection with BB-SAM or 759-SAM.

### In Situ Hybridization and Immunostaining

Paraffin-embedded term human placental sections were deparaffinized in xylene and rehydrated by a series of graded alcohol washes. Control term placental sections were subjected to treatment with RNase A (10 mg/mL, Sigma-Aldrich, R6148) or DNase I (2000 units/mL, New England Biolabs, M0303S) at 37° C. for 30 minutes. OCT-embedded mouse placental sections were dried at 55° C. overnight, rehydrated with 2 PBS washes and treated with proteinase K (15 mg/mL) for 10 minutes. In situ hybridization was performed as previously described21 using 40 nm 50,30 digoxigenin-labeled locked nucleic acid probe for the C19MC hsa-miR-517a/b (Exiqon, 611715-360), positive strand Alu (Qiagen, 339500 LCD0162058-BKG), the C2MC mmu-miR-669a-3p (Qiagen, 339111 YD00616024-BCG), B1 (Qiagen, 339115 YCD0077239-BCG) or scrambled (negative) control (Exiqon, 90005). Hybridization and post-hybridization graded SSC washes were performed at 55° C. The sections were then blocked, and the probes were detected using alkaline phosphatase conjugated sheep anti-digoxigenin Fab fragments (Roche, 11093274910). The signal was developed using NBT/BCIP (Roche, 11697471001) as a substrate, which produces a dark-blue/indigo precipitating dye, followed by nuclear counterstaining with Nuclear Fast Red (Vector Laboratories, H-3403). The sections were dried and covered with mounting medium for image analysis.

Cytokeratin and vimentin immunostaining were performed as previously described21,56,58 using mouse-anti-cytokeratin 7 (Dako M7018, 1:600, RRID:AB_2134589) and chicken-anti-vimentin (Abcam ab39376, RRID:AB_778827, 1 μg/mL) primary antibodies. Biotinylated horse-anti-mouse (Vector Laboratories BA-2000, 3.75 ug/ml, RRID:AB_2313581) secondary antibody with avidinbiotin-peroxidase complex (Vectastain ABC Kit, pk6200, Vector Laboratories) was used to detect cytokeratin and the signal was developed using 3,3-diaminobezidine (sk-4100, Vector Laboratories) as a substrate. To detect vimentin, the donkey-anti-chicken (Jackson ImmunoResearch 703-065-155, 1.2 ug/ml, RRID:AB_2313596) secondary antibody was used with avidin-biotin-alkaline phosphatase (Vectastain ABC-AP, Vector laboratories AK-5200) was used. The signal was developed using Vector Red AP substrate (Vector Red, Vector Laboratories SK-5100) followed by nuclear counterstaining with hematoxylin and mounting for later image analysis.

Immunofluorescent staining was performed as previously described.59 Briefly, term human placental sections were permeabilized with 0.1% TritonX100 at room temperature for 30 minutes and blocked with 3% (w/v) bovine serum albumin for 30 min at room temperature. The sections were then probed with anti-dsRNA mouse monoclonal J2 antibody (Scicons Cat #10010200, RRID:AB_2651015, 1:50) at 40C overnight. After washing with PBS, the sections were probed with donkey anti-mouse IgG (H+L) highly cross-adsorbed secondary antibody, Alexa Fluor_647 (Invitrogen Cat #A-31571, RRID:AB_162542, 2 mg/mL) at room temperature for 30 minutes. After washing with PBS, the sections were probed with Alexa Fluor 488 conjugated HSP60 antibody (Santa Cruz Biotechnology Cat #sc-271215, RRID:AB_10607973, 1:50). Sections were then mounted with Prolong Diamond Antifade Mountant with DAPI (Thermo Fisher Scientific Cat #P36971) nuclei counterstaining and visualized with a FluoView FV1200 inverted laser scanning confocal microscope (Olympus).

### Virus Propagation and Titration

Handling of ZIKV, RSV, and VSV were done according to biosafety level (BSL)-2 guidelines and SARS-COV-2 according to BSL-3 guidelines. VSV Indiana strain, ZIKV PRVABC59 (Puerto Rico strain), and RSV A2 strain were kindly provided by Dr. Michael Teng at the University of South Florida. SARS-COV-2 (Isolate New York 1-PV08001/2020) was obtained from BEI Resources (#NR-52368). VSV and ZIKV were propagated in Vero cells, RSV in Hep-2 cells, and SARS-COV-2 in Vero-E6-ACE2 cells using modified protocols developed from previously described methods.60-63 Briefly, cells were seeded into T-75 or T-175 flasks and allowed to grow until 90% confluency. Cells infected with the respective virus at a multiplicity of infection (MOI) of 0.01-0.1 in low-serum infection media and incubated at 370C/5% CO₂ for 1-2 hours, gently rocking the flasks every 10-15 minutes. At the end of this period, cell culture media was added to the flasks and further incubated for several days. When most cells showed cytopathic effects, cells and supernatant were collected, clarified by centrifugation, and stored at −80_C until used. RSV and ZIKV for in vivo studies were further purified by ultracentrifugation as previously described.61,62

Harvested virus stocks were titrated by plaque assays modified from previously described protocols.60,61,64,65 Briefly, serial 10-fold dilutions of each virus stock were used in duplicate to infect confluent 6-well plates of Vero, Hep-2, or Vero-E6-ACE2 cells for the respective virus as mentioned above in low-serum infection media. After gently rocking the plates every 10-15 minutes while incubating at 37° C./5% CO2 for 1-2 hours, the infection media was removed and 1% agarose overlay media was added. Infected cells were incubated for several days until plaques appeared. The cells were then fixed (with 40% methanol for VSV and ZIKV and 1% formaldehyde for RSV) and stained with dyes (0.4% Crystal violet for VSV and ZIKV and 0.05% neutral red for RSV). Plaques were counted in duplicate per dilution series, and the titer of the virus stock was calculated as plaque-forming units (PFU)/mL. For SARS-COV-2 titration, two layers of solid-overlay media consisting of 2% noble agar were added two days apart, with the second layer containing 0.33% neutral red and directly visualized using a white light transilluminator.65 Plaques were counted, and titer was calculated as PFU/mL.

### In Vitro and In Vivo Viral Infections

After 60 or 72 hour transfection of cells with BB-SAM or 759-SAM, the supernatant from each well was removed and saved at 370C while the cells were infected with VSV (MOI=0.2), ZIKV (MOI=1), RSV (MOI=0.5) or SARS-COV-2 (MOI-0.01) in serum-free media. After 90 minutes, the infection media was replaced with the saved supernatant of each respective well. The cells were collected for RNA extraction after 8 hours infection with VSV or 24 hours with Zika, RSV or SARS-COV-2.

WT and C2MC^{Δ/Δ} mTS cells or mTS cells transfected with IVT control GFP-mRNA, IVT B1-forward RNA, miR-467b-5p- (Life Technologies #MC11605), miR-466b/c/p-3p- (Life Technologies #MC19359) or control-(ThermoFisher Scientific #4464058) mimic for 4 hours were infected with ZIKV (MOI=0.1) in basal media. After 90 minutes, the media was replaced with the supernatant preserved from each well and cells were collected for RNA extraction 24 hours after infection.

For the in vivo ZIKV infections, E8.5 pregnant dams were intraperitoneally (IP) injected with 2.5 mg/mouse anti-IFNAR1 mAb (clone MAR1-5A3, Leinco Technologies Cat #I-401, RRID:AB_2491621). The following day, mice were IP injected with purified ZIKV 104 pfu in 100 μL of sterile 1×PBS. After five days (on E14.5), the dams were sacrificed, and placentas, fetal heads, and the maternal spleens were harvested for total RNA extraction and ZIKV RT-qPCR. Fetal genotypes were determined using the previously described primers17 and confirmed by assessing the expression of C2MC-specific miR-467a in the placentas.

### Small RNA Sequencing and Data Analysis

The small RNA cDNA libraries were generated as previously described, 66 with modifications.67 Briefly, total RNA was extracted from triplicate cultures of AD-293 cells transfected with GFP, 759-SAM or 620-SAM and 293T and DICER1-Ko cells transfected with either BB-SAM or 759-SAM and grown for 72 hours, using the miRNeasy Kit (Qiagen, Cat #217004) and treated with RNase-free DNase (Qiagen, Cat #79254) according to the manufacturer's instructions.

For AD-293 cells, 2 mg of total RNA were converted into a small RNA (sRNA) cDNA library according to published protocol.66 The RNA input for each sample was ligated to a 30 adaptor barcoded sequence, pooled, size selected, and gel purified, followed by 50 adapter ligation and then subjected to size selection and gel purification. SuperScript III was used for second strand synthesis and the cDNA library preparation was completed with alkaline RNA hydrolysis and PCR amplification for 10 cycles.

For 293T and DICER-Ko cells, the RNA were organized in one batch of 12 samples, each containing 1.0 mg total RNA. For cDNA library preparation, 2.5 fmoles of Calibrator Set2 (standard calibrator), a set of ten 21-nt 5′-phosphorylated RNA oligos, was added. Each RNA sample was individually 3′-adapter-ligated. Up to 24 reaction products were pooled, 5′ adapter-ligated, PCR-amplified and sequenced in a single NextSeq500 lane. Reads were demultiplexed, mapped against a curated hg19-based miRNA reference-transcriptome, sorted, and tabulated into different RNA categories. Sequencing data were processed (Illumina software suite), followed by read extraction using an in-house RNA Sequencing Data Analysis Pipeline (RSDAP) specifying a size range of 16 to 45 nt and default parameters. Demultiplexed RNA sequencing data was mapped against our curated human reference transcriptome to obtain miRNA raw read and read frequency profiles and abundance of fragments of other RNA classes, such as tRNAs, snRNAs, scRNAs, and TRNAs. Mapped data were used to generate RNA summary tables, as well as detailed miRNA raw read and read frequency Tables that were used for differential expression analysis and unsupervised clustering, respectively.

Reads annotated as calibrator, expression system (plasmid & E. coli) marker and adapter were considered as reads of technical origin; those remaining were considered as reads derived from the sample. For differential expression analyses, tabulated shared raw reads of merged miRNAs reported by RSDAP were used to perform differential expression analyses using DESeq2,47 considering only miRNAs with at least five counts across all samples. Selected metadata categories (GFP, 759-SAM, 620-SAM and BB-SAM, 759-SAM), were used as experimental design parameters, comparing miRNA abundance differences between two distinctive sample groups. For all merged miRNAs, we considered a differential change in abundance as detected if the underlying base mean was at least 5 normalized counts and as statistically significant if the reported adjusted p-value was less than 0.05.

### RNA Sequencing and Bioinformatics Analysis

mRNA libraries of AD-293 cells were prepared by utilizing the Illumina TruSeq Stranded mRNA LT protocol using 500 ng total RNA and NEB's Protoscript II reverse transcriptase for the first-strand cDNA synthesis according to the manufacturer's protocol. Individual RNAseq libraries were quality controlled on an Agilent TapeStation with a High Sensitivity D1000 ScreenTape. Indexed samples were quantified using the Qubit dsDNA HS assay and were pooled at equimolar concentration (10 nM). The libraries were sequenced on an Illumina NextSeq. 500 sequencer 75-bp paired-end in mid-output mode in the Genomics Core Facility of The Rockefeller University.

From 293T and DICER-Ko cells, 0.1 mg total RNA was used for stranded total RNA library preparations (Illumina TruSeq, Cat #_20020596), and this workflow included a Ribo-Zero Human/Mouse/Rat RNA depletion step. Libraries were prepared with unique barcodes and pooled at equalmolar ratios. The pool was denatured and sequenced on Illumina NextSeq 500 sequencer using high output V2 reagents and NextSeq Control Software v1.4 to generate 75 bp paired-end reads, following manufacture's protocol (Cat #15048776 Rev.E). mRNA sequencing reads were aligned to the human genome (GRChg38) using the RNASTAR aligner48 allowing for two mismatches. Read counts were generated using featureCounts,49 and differential expression analysis was completed using edgeR.50 Differentially expressed genes were considered significant with an FDR <0.1 and fold-change >2.0 up or down.

Gene set enrichment analysis was performed using the UC San Diego and Broad Institute GSEA software and Molecular Signatures Database (MSigDB).51

### Sample Size Calculation

All in vitro experiments were performed in triplicate. A representative experiment of a minimum of three independent experiments is shown in the manuscript, unless indicated otherwise in the Figure legends. Sample size for in vivo experiments was calculated using IBM SPSS statics software (version 28) to achieve a power of 0.8 at p=0.05. A preliminary experiment with the fetal placentas of one dam was used to obtain the mean and standard deviation values to calculate the minimum number of samples needed per experimental group.

### Statistical Analysis

Parametric (two-tailed unpaired t-test with or without Welch's correction and one-way ANOVA with Dunnett's or Tukey's post hoc tests) and non-parametric (Mann-Whitney U and Kruskall Wallis) tests were used where appropriate for statistical analysis of the data using GraphPad Prism 9 software. *P<0.05 was considered to be statistically significant. The investigators were not blinded to the experimental conditions. All data represent the mean±SEM (n=3) of a representative of at least three independent experiments unless otherwise noted in the relevant Figure legends.

### Tables

TABLE 1
Differentially expressed miRNA 759-SAM or 620-SAM vs GFP detected in AD-293 cells (see FIGS. 1 and 7)

| AD-293: gRNA759 vs. GFP | | | | AD-293: gRNA620 vs. GFP | | | |
|---|---|---|---|---|---|---|---|
| C19MC- | | | | C19MC- | | | |
| member | Differential | Other | Differential | member | Differential | Other | Differential |
| miRNAs | expression | miRNAs | expression | miRNAs | expression | miRNAs | expression |
| hsa-miR- | Upregulation | hsa-miR- | Upregulation | hsa-miR- | Upregulation | hsa-miR- | Upregulation |
| 520a(1) | | 654(1) | | 520f(1) | | 135b(1) | |
| hsa-miR- | Upregulation | hsa-miR- | Upregulation | hsa-miR- | Upregulation | hsa-miR- | Upregulation |
| 520f(1) | | 1270(2) | | 498(1) | | 1(2) | |
| hsa-miR- | Upregulation | hsa-miR- | Upregulation | hsa-miR- | Upregulation | hsa-miR- | Upregulation |
| 498(1) | | 514a(3) | | 516b(2) | | 184(1) | |
| hsa-miR- | Upregulation | hsa-miR- | Upregulation | hsa-miR- | Upregulation | hsa-miR- | Upregulation |
| 516b(2) | | 139(1) | | 520a(1) | | 1294(1) | |
| hsa-miR- | Upregulation | hsa-miR- | Upregulation | hsa-miR- | Upregulation | hsa-miR- | Upregulation |
| 524(1) | | 302a-5p(1) | | 520d(1) | | 3934(1) | |
| hsa-miR- | Upregulation | hsa-miR- | Upregulation | hsa-miR- | Upregulation | hsa-miR- | Downregulation |
| 518c-3p(1) | | 381(1) | | 520g(2) | | 4473-3p(1) | |
| hsa-miR- | Upregulation | hsa-miR- | Upregulation | hsa-miR- | Upregulation | | |
| 518b-3p(1) | | 1294(1) | | 524(1) | | | |
| hsa-miR- | Upregulation | hsa-miR- | Upregulation | hsa-miR- | Upregulation | | |
| 520d(1) | | 3934(1) | | 520b(2) | | | |
| hsa-miR- | Upregulation | hsa-miR- | Downregulation | hsa-miR- | Upregulation | | |
| 520b(2) | | 33b(1) | | 519c(1) | | | |
| hsa-miR- | Upregulation | hsa-miR- | Downregulation | hsa-miR- | Upregulation | | |
| 515(2) | | 205(1) | | 518c-3p(1) | | | |
| hsa-miR- | Upregulation | hsa-miR- | Downregulation | hsa-miR- | Upregulation | | |
| 519c(1) | | 144(1) | | 518b-3p(1) | | | |
| hsa-miR- | Upregulation | | | hsa-miR- | Upregulation | | |
| 525-5p(1) | | | | 515(2) | | | |
| hsa-miR- | Upregulation | | | hsa-miR- | Upregulation | | |
| 518f-3p(1) | | | | 518d-5p(5) | | | |
| hsa-miR- | Upregulation | | | hsa-miR- | Upregulation | | |
| 526b-5p(1) | | | | 518f-3p(1) | | | |
| hsa-miR- | Upregulation | | | hsa-miR- | Upregulation | | |
| 1283-5p(2) | | | | 526b-5p(1) | | | |
| hsa-miR- | Upregulation | | | hsa-miR- | Upregulation | | |
| 518d-5p(5) | | | | 517b(1) | | | |
| hsa-miR- | Upregulation | | | hsa-miR- | Upregulation | | |
| 520g(2) | | | | 525-5p(1) | | | |
| hsa-miR- | Upregulation | | | hsa-miR- | Upregulation | | |
| 519b(1) | | | | 1283-5p(2) | | | |
| hsa-miR- | Upregulation | | | hsa-miR- | Upregulation | | |
| 517b(1) | | | | 520e(1) | | | |
| hsa-miR- | Upregulation | | | hsa-miR- | Upregulation | | |
| 518a-3p(2) | | | | 518e-3p(1) | | | |
| hsa-miR- | Upregulation | | | hsa-miR- | Upregulation | | |
| 516a(2) | | | | 518a-3p(2) | | | |
| hsa-miR- | Upregulation | | | hsa-miR- | Upregulation | | |
| 512-3p(2) | | | | 516a(2) | | | |
| hsa-miR- | Upregulation | | | hsa-miR- | Upregulation | | |
| 518e-3p(1) | | | | 519b(1) | | | |
| hsa-miR- | Upregulation | | | hsa-miR- | Upregulation | | |
| 523-3p(1) | | | | 517a(2) | | | |
| hsa-miR- | Upregulation | | | hsa-miR- | Upregulation | | |
| 518a-5p(3) | | | | 519d(1) | | | |
| hsa-miR- | Upregulation | | | hsa-miR- | Upregulation | | |
| 517a(2) | | | | 522-3p(1) | | | |
| hsa-miR- | Upregulation | | | hsa-miR- | Upregulation | | |
| 519d(1) | | | | 518a-5p(3) | | | |
| hsa-miR- | Upregulation | | | hsa-miR- | Upregulation | | |
| 519a(2) | | | | 1323(1) | | | |
| hsa-miR- | Upregulation | | | | | | |
| 1323(1) | | | | | | | |
| hsa-miR- | Upregulation | | | | | | |
| 520e(1) | | | | | | | |

TABLE 2
Differentially expressed genes in 759-SAM vs BB-SAM
transfected 293T cells (see FIGS. 2 and 8).
Sheet “miRNA 759-SAM vs BB-SAM in 293T cells”

| C19MC-member | Differential | | Differential |
|---|---|---|---|
| miRNAs | expression | Other miRNAs | expression |
| hsa-miR-520f(1) | Upregulation | hsa-miR-130a(1) | Upregulation |
| hsa-miR-1323(1) | Upregulation | hsa-miR-200b(1) | Upregulation |
| hsa-miR-517a(2) | Upregulation | hsa-miR-381(1) | Upregulation |
| hsa-miR-515(2) | Upregulation | hsa-miR-654(1) | Upregulation |
| hsa-miR-520b(2) | Upregulation | hsa-miR-376c(1) | Upregulation |
| hsa-miR-524(1) | Upregulation | hsa-miR-1185-1-3p(1) | Upregulation |
| hsa-miR-518b-3p(1) | Upregulation | hsa-miR-885(1) | Upregulation |
| hsa-miR-518c-3p(1) | Upregulation | hsa-miR-487b(1) | Upregulation |
| hsa-miR-520a(1) | Upregulation | hsa-miR-377(1) | Upregulation |
| hsa-miR-520d(1) | Upregulation | hsa-miR-409-3p(1) | Upregulation |
| hsa-miR-519d(1) | Upregulation | hsa-miR-127(1) | Upregulation |
| hsa-miR-525-5p(1) | Upregulation | hsa-miR-655(1) | Upregulation |
| hsa-miR-498(1) | Upregulation | hsa-miR-208a(1) | Upregulation |
| hsa-miR-526b-5p(1) | Upregulation | hsa-miR-376a-3p(2) | Upregulation |
| hsa-miR-520g(2) | Upregulation | hsa-miR-889(1) | Upregulation |
| hsa-miR-520e(1) | Upregulation | | |
| hsa-miR-518f-3p(1) | Upregulation | | |
| hsa-miR-519c(1) | Upregulation | | |
| hsa-miR-512-3p(2) | Upregulation | | |
| hsa-miR-518d-5p(5) | Upregulation | | |
| hsa-miR-516b(2) | Upregulation | | |
| hsa-miR-1283-5p(2) | Upregulation | | |
| hsa-miR-517b(1) | Upregulation | | |
| hsa-miR-518a-5p(3) | Upregulation | | |
| hsa-miR-516a(2) | Upregulation | | |
| hsa-miR-521(2) | Upregulation | | |
| hsa-miR-523-3p(1) | Upregulation | | |
| hsa-miR-519b(1) | Upregulation | | |
| hsa-miR-519e-5p(1) | Upregulation | | |

TABLE 3
Differentially expressed genes in 759-SAM vs BB-SAM
transfected DICER-Ko (see FIGS. 2 and 9)
Sheet “miRNA 759-SAM vs BB-SAM in DICER-Ko cells

| | | | |
|---|---|---|---|
| C19MC-member | Differential | | Differential |
| miRNAs | expression | Other miRNAs | expression |

(None were detected with significant expression changes)

TABLE 4
Alu SINEs located in CYP19A1 gene on chromosome 15,
genome region chr15: 51,208,057-51,338,596 of the GRCh38/hg38
human genome assembly (see FIGS. 3 and 10).
Table S4. Alu SINEs located in CYP19A1 gene on chromosome 15,
genome region chr15: 51,208,057-51,338,596 of the GRCh38/hg38
human genome assembly.

| | | | | | Rep | Rep | Rep | | |
|---|---|---|---|---|---|---|---|---|---|
| genoName | genoStart | genoEnd | genoLeft | strand | Name | Class | Family | repStart | repEnd |
| chr15 | 51216247 | 51216552 | −50774637 | − | AluSx1 | SINE | Alu | −11 | 301 |
| chr15 | 51225820 | 51226116 | −50765073 | + | AluY | SINE | Alu | 14 | 309 |
| chr15 | 51227394 | 51227672 | −50763517 | + | AluSz6 | SINE | Alu | 11 | 282 |
| chr15 | 51229247 | 51229405 | −50761784 | + | AluJb | SINE | Alu | 141 | 298 |
| chr15 | 51230614 | 51230920 | −50760269 | − | AluSx1 | SINE | Alu | −8 | 304 |
| chr15 | 51236370 | 51236428 | −50754761 | − | AluJo | SINE | Alu | −15 | 297 |
| chr15 | 51238498 | 51238652 | −50752537 | − | AluSz6 | SINE | Alu | −22 | 290 |
| chr15 | 51247457 | 51247718 | −50743471 | − | AluSz | SINE | Alu | 0 | 312 |
| chr15 | 51248933 | 51249240 | −50741949 | − | AluSx1 | SINE | Alu | −14 | 298 |
| chr15 | 51272966 | 51273248 | −50717941 | − | AluSx3 | SINE | Alu | −20 | 291 |
| chr15 | 51273724 | 51274004 | −50717185 | + | AluSx1 | SINE | Alu | 2 | 280 |
| chr15 | 51280114 | 51280397 | −50710792 | − | AluY | SINE | Alu | −5 | 306 |
| chr15 | 51304890 | 51305199 | −50685990 | − | AluSq2 | SINE | Alu | 0 | 313 |
| chr15 | 51305560 | 51305854 | −50685335 | − | AluSx | SINE | Alu | −12 | 300 |
| chr15 | 51313514 | 51313811 | −50677378 | + | AluSx1 | SINE | Alu | 1 | 297 |
| chr15 | 51316207 | 51316338 | −50674851 | + | AluJo | SINE | Alu | 1 | 132 |
| chr15 | 51316596 | 51316728 | −50674461 | + | FLAM_C | SINE | Alu | 1 | 131 |
| chr15 | 51317101 | 51317407 | −50673782 | − | AluY | SINE | Alu | −10 | 301 |
| chr15 | 51325091 | 51325188 | −50666001 | + | FLAM_A | SINE | Alu | 20 | 121 |
| chr15 | 51325557 | 51325864 | −50665325 | + | AluSc5 | SINE | Alu | 1 | 307 |
| chr15 | 51327486 | 51327591 | −50663598 | − | AluSz6 | SINE | Alu | −5 | 307 |

TABLE 5
Sequences and BLAT alignment of fl-Alu RT-PCR products in Hela cells
exposed to heat shock. Sequences and BLAT alignment (human GRCh38/hg38) of single
colonies of fl-Alu RT-PCR products after gel purification and cloning into TOPO TA vectors
obtained from Hela cells subjected to heat shock (n = 20). Each clone was sequenced in both
directions using the T7 and T3 primers. Forward and reserve primers are indicated in single
underline (red) and double underline (blue), respectively. Sequences that did not include
EcoRI restriction sites or the PCR primer sequences are indicated as bad sequencing. “N”
in the sequences is understood to be any nucleotide.

| | | | | hg38/ |
|---|---|---|---|---|
| Clone | | | | Human |
| number | Sequencing with T3 primer | Sequencing with T7 primer | Span | Alu |
| Heat | Bad sequencing | GAATTCGCCCTTGTGCGGTGGCAC | 217 | AluSx |
| shock_1 | | ACGCTTGTAATCCCAGCACTTTGG | | |
| | | GAGGTCGAGACGGGTGGATCACC | | |
| | | TCAGGTCAGAGTTCAAGAGCAGCC | | |
| | | CCGCCATCAAGACAAAACCTCCTC | | |
| | | TCTACTAAAAATGCAAAATATAGCTA | | |
| | | GGCGTGGTGGTACACACCTGTAGT | | |
| | | CTCAGCTACGTGGGAGGCGGCAG | | |
| | | CAGGAGAATCTCTTGAACCCGTGA | | |
| | | GAAGGAGATTGCAAGGGCGAATTC | | |
| | | SEQ ID NO: 43 | | |
| Heat | GAATTCGCCCTTGCANTCTCCTT | GAATTCGCCCTTCCGGGTGCGGTG | 217 | AluSx |
| shock_2 | CTCACGGGTTCAAGAGATTCTCC | GCACACGCTTGTAATCCCAGCACT | | |
| | TGCTGCCGCCTCCCACGTAGCT | TTGGGAGGTCGAGACGGGTGGATC | | |
| | GAGACTACAGGTGTGTACTACCA | ACCTCAGGTCAGAGTTCAAGAGCA | | |
| | CGCCTAGCTATATTTTGCATTTTTA | GCCCCGCCATCAAGACAAAACCT | | |
| | GTAGAGAGGAGGTTTTGTCTTGA | CCTCTCTACTAAAAATGCAAAATATA | | |
| | TGGCGGGGCTGCTCTTGAACTC | GCTAGGCGTGGTAGTACACACCTG | | |
| | TGACCTGAGGTGATCCACCCGT | TAGTCTCAGCTACGTGGGAGGCGG | | |
| | CTCGACCTCCCAAAGTGCTGGG | CAGCAGGAGAATCTCTTGAACCCG | | |
| | ATTACAAGCGTGTGCCACCGCA | TGAGAAGGAGATTGCAAGGGCGAA | | |
| | CCCGGAAGGGCGAATTC | TTC | | |
| | SEQ ID NO: 44 | SEQ ID NO: 45 | | |
| Heat | GAATTCGCCCTTGCATNCTCCTT | GAATTCGCCCTTGGTGCGGTGGCA | 218 | AluSx |
| shock_3 | CTCACGGGTTCAAGAGATTCTCC | CACGCTTGTAATCCCAGCACTTTGG | | |
| | TGCTGCCGCCTCCCACGTAGTC | GAGGTCGAGACGGGTGGATCACCT | | |
| | TGAGACTACAGGTGTGTACCAC | CAGGTCAGAGTTCAAGAGCAGCCC | | |
| | CACGCCTAGCTATATTTTGCATTT | CGCCATCAAGATAAAACCTCCTCT | | |
| | TTAGTAGAGAGGAGGTTTTATCTT | CTACTAAAAATGCAAAATATAGCTA | | |
| | GATGGCGGGGCTGCTCTTGAAC | GGCGTGGTGGTACACACCTGTAGT | | |
| | TCTGACCTGAGGTGATCCACCC | CTCAGCTACGTGGGAGGCGGCAG | | |
| | GTCTCGACCTCCCAAAGTGCTG | CAGGAGAATCTCTTGAACCCGTGA | | |
| | GGATTACAAGCGTGTGCCACCG | GAAGGAGATTGCAAGGGCGAATTC | | |
| | CACCAAGGGCGAATTC | SEQ ID NO: 47 | | |
| | SEQ ID NO: 46 | | | |
| Heat | GATTCCCTTGCAATNCTCCTTCT | GAATTCGCCCTTCCGGGTGCGGTG | 221 | AluSx |
| shock_4 | CACGGGTTCAAGAGATTCTCCTG | GCACACGCTTGTAATCCCAGCACT | | |
| | CTGCCGCCTCCCACGCAGCTG | TTGGGAGGTCGAGACGGGCGGAT | | |
| | AGACTACAGGTGTGTACCACCA | CACCTCAGGTCAGAGTTCAAGAGC | | |
| | CGCCTAGCTATATTTTGCATTTTTA | AGCCCCGCCATCAAGACAAAACCT | | |
| | GTAGAGAGGAGGTTTTGTCTTGA | CCTCTCTACTAAAAATGCAAAATATA | | |
| | TGGCGGGGCTGCTCTTGAACTC | GCTAGGCGTGGTGGTACACACCTG | | |
| | TGACCTGAGGTGATCCGCCCGC | TAGTCTCAGCTACGTGGGAGGCGG | | |
| | CTCGACCTCCCAAGCTGCTGTT | CAGCAGGAGAATCTCTTGAACCCG | | |
| | ATTACGATCCCGTGCCACCGCC | TGAGAAGGAGATTGCAAGGGCGAA | | |
| | ATAGTGAGGACGACTCCGCCTC | TTC | | |
| | AACCTTCAATTC | SEQ ID NO: 49 | | |
| | SEQ ID NO: 48 | | | |
| Heat | Bad sequencing | GAATTCGCCCTTGGGTGCGGTGGC | 214 | AluSx3 |
| shock_5 | | ACACGCTTGTAATCCCAGCACTITG | | |
| | | GGAGGCCAAGGCGGGCAGATCAT | | |
| | | GAGGTCAGGAAATTGAGACCAGCC | | |
| | | TGGCTAACATGGTGAAACTCTGTCT | | |
| | | CTACTAAAAATACAGAAAATTAGCT | | |
| | | GGGCATGGTGGCCAGCGCCTGTA | | |
| | | GTCCCAGGTACTTGGAGGCTGAGG | | |
| | | CAGGAGAATTGCTTGAACCCGTGA | | |
| | | GAAGGAGATTGCAAGGGCGAATTC | | |
| | | SEQ ID NO: 50 | | |
| Heat | GAATTCGCCCTTGCANTCTCCTT | GAATTCGCCCTTCCGGGTGCGGTG | 73 | AluSx |
| shock_6 | CTCACGGGTTCAAGCGATTCTC | GCACACGCTTGTGGTCCCAGCTAT | | |
| | CTGCCTCAGCCTCCTGAATAGC | TCAGGAGGCTGAGGCCAGGAGAA | | |
| | TGGGACCACAAGCGTGTGCCAC | TCGCTTGAACCCGTGAGAAGGAGA | | |
| | CGCACCC GGAAGGGCGAATTC | TTG CAAGGGCGAATTC | | |
| | SEQ ID NO: 51 | SEQ ID NO: 52 | | |
| Heat | GAATTCGCCCTTGCANTCTCCTT | GAATTCGCCCTTCCGGGTGCGGTG | 203 | AluSc |
| shock_7 | CTCACGGGTTCAAGCGATTCTC | GCACACGCTTGTAATCCCAGCACT | | |
| | CTGCCTCAGCCTCCCAACTAAC | TTGGGAGGTCGAGGCCGGGCGGA | | |
| | TGGGACTACAAGCGCGCGCCA | TCACGAGGTCAGGAGATCGAGACC | | |
| | CCACGCCCAGCTAATTTTTGTATT | ATCCTGGCCAACATGGTGAAGCCC | | |
| | TTTAGTAGAGACGGGGCTTCACC | CGTCTCTACTAAAAATACAAAAATTA | | |
| | ATGTTGGCCAGGATGGTCTCGAT | GCTGGGCGTGGTGGCGCGCGCTT | | |
| | CTCCTGACCTCGTGATCCGCCC | GTAGTCCCAGTTAGTTGGGAGGCT | | |
| | GCCTCGACCTCCCAAAGTGCTG | GAGGCAGGAGAATCGCTTGAACCC | | |
| | GGATTACAAGCGTGTGCCACCG | GTGAGAAGGAGATTGCAAGGGCGA | | |
| | CACCCGGAAGGGCGAATTC | ATTC | | |
| | SEQ ID NO: 53 | SEQ ID NO: 54 | | |
| Heat | GAATTCGCCCTTGCANTCTCCTT | GAATTCGCCCTTCCGGGTGCGGTG | 221 | AluSx |
| shock_8 | CTCACGGGTTCAAGAGATCCTC | GCACACGCTTGTAATCCCAGCACT | | |
| | CTGCTGCCGCCTCCCACGTAGC | TTGGGAGGTCGAGACGGGTGGATC | | |
| | TGAGACTACAGGTGTGTACCAC | ACCTCAGGTCAGAGTCCAAGAGCA | | |
| | CACGCCTAGCTATATTTTGCATTT | GCCCCGCCATCAAGACAAAACCTC | | |
| | TTAGTAGAGAGGAGGTTTTGTCTT | CTCTCTACTAAAAATGCAAAATATAG | | |
| | GATGGCGGGGCTGCTCTTGGAC | CTAGGCGTGGTGGTACACACCTGT | | |
| | TCTGACCTGAGGTGATCCACCC | AGTCTCAGCTACGTGGGAGGCGG | | |
| | GTCTCGACCTCCCAAAGTGCTG | CAGCAGGAGGATCTCTTGAACCCG | | |
| | GGATTACAAGCGTGTGCCACCG | TGAGAAGGAGATTGCAAGGGCGAA | | |
| | CACCCGGAAGGGCGAATTC | TTC | | |
| | SEQ ID NO: 55 | SEQ ID NO: 56 | | |
| Heat | GAATTCGCCCTTCGTGCGGTGG | GAATTCGCCCTTGCANTCTCCTTCT | 217 | AluSx |
| shock_9 | CACACGCTTGTAATCCCAGCAC | CACGGGTTCAAGAGATTCTCCTGCT | | |
| | TTTGGGAGGTCGAGACGGGTGG | GCCGCCTCCCACGTAGTCTGAGA | | |
| | ATCGCCTCAGGTCAGAGTTCAA | CTACAGGTGTGAACCACCACGCCT | | |
| | GAGCAGCCCCGCCATCAAGAC | AGCTATATTTTGCATTTTTAGTAGAGA | | |
| | AAAACCTCCTCTCTACTAAAAAT | GGAGGTTTTGTCTTGATGGCGGGG | | |
| | GCAAAATATAGCTAGGCGTGGTG | CTGCTCTTGAACTCTGACCTGAGG | | |
| | GTTCACACCTGTAGTCTCAGCTA | CGATCCACCCGTCTCGACCTCCCA | | |
| | CGTGGGAGGCGGCAGCAGGAG | AAGTGCTGGGATTACAAGCGTGTG | | |
| | AATCTCTTGAACCCGTGAGAAGG | CCACCGCACGAAGGGCGAATTC | | |
| | AGATTGCAAGGGCGAA | SEQ ID NO: 58 | | |
| | TTC | | | |
| | SEQ ID NO: 57 | | | |
| Heat | GAATTCGCCCTTGGGTGCGGTG | AATTCGCCCTTGCANTCTCCTTCTC | 173 | AluSx4 |
| shock_10 | GCACACGCTTGTAATACCAGCA | ACGGGTTCAAGCTATTCTCCTGCCT | | |
| | CTTTGGGAGGCCAAGGTGGGCG | CAGCCTCCCGAGTAGCTGGGACT | | |
| | GATCACCTGAGGTCAGGAGTTC | ACAAGGGATCTGCCACCACGCCC | | |
| | AAGACCAGCCTGGCCAATATGG | GGCTAATTTTTGTAATTTTAGTAGAG | | |
| | TGAAAGTCCGTCTCTACTAAAATT | ACGGACTTTCACCATATTGGCCAG | | |
| | ACAAAAATTAGCCGGGCGTGGT | GCTGGTCTTGAACTCCTGACCTCA | | |
| | GGCAGATCCCTTGTAGTCCCAG | GGTGATCCGCCCACCTTGGCCTC | | |
| | CTACTCGGGAGGCTGAGGCAG | CCAAAGTGCTGGTATTACAAGCGT | | |
| | GAGAATAGCTTGAACCCGTGAG | GTGCCACCGCACCAAGGGCGAAT | | |
| | AAGGAGATTGCAAGGGC | TC | | |
| | GAATTC | SEQ ID NO: 60 | | |
| | SEQ ID NO: 59 | | | |
| Heat | GAATTCGCCCTTGCAATCTCCTT | GAATTCGCCCTTCCGGGTGCGGTG | 221 | AluSx |
| shock_11 | CTCACGGGTTCAAGAGACTCTC | GCACACGCTTGTAATCCCAGCACT | | |
| | CTGCTGCCGCCTCCCACGTAGC | TTGGGAGGTCGAGACGGGTGGATC | | |
| | TGAGACTACAGGTGTGTACCAC | ACCTCAGGTCAGAGTTCAAGAGCA | | |
| | CACGCCTAGCTATATTTTGCATTT | GCCCCGCCATCAAGACAAAACCT | | |
| | TTAGTAGAGAGGAGGTTTTGTCTT | CCTCTCTACTAAAAATGCAAAATATA | | |
| | GATGGCGGGGCTGCTCTTGAAC | GCTAGGCGTGGTGGTACACACCTG | | |
| | TCTGACCTGAGGTGATCCACCC | TAGTCTCAGCTACGTGGGAGGCGG | | |
| | GTCTCGACCTCCCAAAGTGCTG | CAGCAGGAGAGTCTCTTGAACCCG | | |
| | GGATTACAAGCGTGTGCCACCG | TGAGAAGGAGATTGCAAGGGCGAA | | |
| | CACCCGGAAGGGCGA | TTC | | |
| | ATTC | SEQ ID NO: 62 | | |
| | SEQ ID NO: 61 | | | |
| Heat | GAATTCGCCCTTCCGGGTGCGG | ATTCGCCCTTGCANTCTCCTTCTCA | 221 | AluSx |
| shock_12 | TGGCACACGCTTGTAATCCCAG | CGGGTTCAAGAGATTCTCCTGCTG | | |
| | CACTTTGGGAGGTCGAGACGGG | CCGCCTCCCACGTAGCTGAGACTA | | |
| | TGGATCACCTCAGGTCAGAGTTC | CAGGTGTGTACCACCACGCCTAGC | | |
| | AAGAGCAGCCCCGCCATCAAGA | TATATTTTGCATTTTTAGTAGAGAGGA | | |
| | CAAAACCTCCTCTCTACTAAAAA | GGTTTTGTCTTGATGGCGGGGCTG | | |
| | TGCAAAATATAGCTAGGCGTGGT | CTCTTGAACTCTGACCTGAGGTGAT | | |
| | GGTACACACCTGTAGTCTCAGCT | CCACCCGTCTCGACCTCCCAAAGT | | |
| | ACGTGGGAGGCGGCAGCAGGA | GCTGGGATTACAAGCGTGTGCCAC | | |
| | GAATCTCTTGAACCCGTGAGAAG | CGCACCCGGAAGGGCGAATTC | | |
| | GAGATTGCAAGGGC | SEQ ID NO: 64 | | |
| | GAATTC | | | |
| | SEQ ID NO: 63 | | | |
| Heat | Bad sequencing | GAATTCGCCCTTGCAATCTCCTTCT | 374 | AluSx |
| shock_13 | | CACGGGTTCAAGCGATTCTCCTGC | | |
| | | CTCAGCCTCCTGAGTAGCTGGGAT | | |
| | | TACAAGCGTGTGCCACCGCACCC | | |
| | | GGAAGGGCGAATTC | | |
| | | SEQ ID NO: 65 | | |
| Heat | GAATTCGCCCTTGCAATCTCCTT | GAATTCGCCCTTCCGGGTGCGGTG | 221 | AluSx |
| shock_14 | CTCACGGGTTCAAGAGATTCTCC | GCACACGCTTGTAATCCCAGCACT | | |
| | TGCTGCCGCCTCCCACGTAGCT | TTGGGAGGTCGAGACGGGTGGATC | | |
| | GAGACTACAGGTGTGTACCACC | ACCTCAGGTCAGAGTTCGAGAGCA | | |
| | ACGCCTAGCTATATTTTGCATTTTT | GCCCCGCCATCAAGACAAAACCTC | | |
| | AGTAGAGAGGAGGTTTTGTCTTG | CTCTCTACTAAAAATGCAAAATATAG | | |
| | ATGGCGGGGCTGCTCTCGAACT | CTAGGCGTGGTGGTACACACCTGT | | |
| | CTGACCTGAGGTGATCCACCCG | AGTCTCAGCTACGTGGGAGGCGG | | |
| | TCTCGACCTCCCAAAGTGCTGG | CAGCAGGAGAATCTCTTGAACCCG | | |
| | GATTACAAGCGTGTGCCACCGC | TGAGAAGGAGATTGCAAGGGCGAA | | |
| | ACCCGGAAGGGCGAATTC | TTC | | |
| | SEQ ID NO: 66 | SEQ ID NO: 67 | | |
| Heat | GAATTCGCCCTTGCAATCTCCTT | ATTCGCCCTTCCGGGTGCGGTGGC | 221 | AluSx |
| shock_15 | CTCACGGGTTCAAGAGATTCTCC | ACACGCTTGTAATCCCAGCACTTTG | | |
| | TGCTGCCGCCTCCCACGTAGCT | GGAGGCCGAGACGGGTGGATCAC | | |
| | GAGACTACAGGTGTGTACCACC | CTCAGGTCAGAGTTCAAGAGCAGC | | |
| | ACGCCTAGCTATATTTTGCATTTTT | CCCGCCATCAAGACAAAACCTCCT | | |
| | AGTAGAGAGGAGGTTTTGTCTTG | CTCTACTAAAAATGCAAAATATAGCT | | |
| | ATGGCGGGGCTGCTCTTGAACT | AGGCGTGGTGGTACACACCTGTAG | | |
| | CTGACCTGAGGTGATCCACCCG | TCTCAGCTACGTGGGAGGCGGCA | | |
| | TCTCGGCCTCCCAAAGTGCTGG | GCAGGAGAATCTCTTGAACCCGTG | | |
| | GATTACAAGCGTGTGCCACCGC | AGAAGGAGATTGCAAGGGCGAATT | | |
| | ACCCGGAAGGGCGAATTC | C | | |
| | SEQ ID NO: 68 | SEQ ID NO: 69 | | |
| Heat | GAATTCGCCCTTGGGTGCGGTG | ATTCGCCCTTGCAATCTCCTTCTCA | 219 | AluSx1 |
| shock_16 | GCACACGCTTGTAATCCCAGCA | CGGGTTCAAGCGATTCTCCTGCCT | | |
| | CTTTGGGAGGCCGAGGTGAGCG | CAGTCTCCCGAGTAGCTGGTATTA | | |
| | GATCATGAGGTCAGGAGTTTGAG | CAGGCGCCTGCCACCATGCCCAG | | |
| | ACCAGCCTGGCCAACACAGTGA | CTAATTTTTGTACTTTTAGTAGACGG | | |
| | AAACCCGTCTACTAAAAGTACAA | GTTTTCACTGTGTTGGCCAGGCTGG | | |
| | AAATTAGCTGGGCATGGTGGCAG | TCTCAAACTCCTGACCTCATGATCC | | |
| | GCGCCTGTAATACCAGCTACTC | GCTCACCTCGGCCTCCCAAAGTG | | |
| | GGGAGACTGAGGCAGGAGAATC | CTGGGATTACAAGCGTGTGCCACC | | |
| | GCTTGAACCCGTGAGAAGGAGA | GCACCAAGGGCGAATTC | | |
| | TTGCAAGGGCGAATTC | SEQ ID NO: 71 | | |
| | SEQ ID NO: 70 | | | |
| Heat | GAATTCGCCCTTGCANTCTCCTT | ATTCGCCCTTGGGTGCGGTGGCAC | 198 | AluSc |
| shock_17 | CTCACGGGTTCAAGCGATTCTCC | ACGCTTGTAATCCCAGCACTTTGG | | |
| | TGCCTCAGCCTCCCAACTAACTG | GAGGTCGAGGCGGGCGGATCACG | | |
| | GGACTACAAGCGCGCGCCACC | AGGTCAGGAGATCGAGACCATCCT | | |
| | ACGCCCAGCTAATTTTTGTATTTTT | GGCCAACATGGTGAAGCCCCGTCT | | |
| | AGTAGAGACGGGGCTTCACCAT | CTACTAAAAATACAAAAATTAGCTG | | |
| | GTTGGCCAGGATGGTCTCGATCT | GGCGTGGTGGCGCGCGCTTGTAG | | |
| | CCTGACCTCGTGATCCGCCCGC | TCCCAGTTAGTTGGGAGGCTGAGG | | |
| | CTCGACCTCCCAAAGTGCTGGG | CAGGAGAATCGCTTGAACCCGTGA | | |
| | ATTACAAGCGTGTGCCACCGCA | GAAGGAGATTGCAAGGGCGAATTC | | |
| | CCCAAGGGCGAATTC | SEQ ID NO: 73 | | |
| | SEQ ID NO: 72 | | | |
| Heat | GAATTCGCCCTTGCAATCTCCTT | GAATTCGCCCTTGGGTGCGGTGGC | 219 | AluSx |
| shock_18 | CTCACGGGTTCAAGAGATTCTCC | ACACGCTTGTAATCCCAGCACTTTG | | |
| | TGCTGCCGCCTCCCACGTAGCT | GGAGGTCGAGACGGGTGGATCAC | | |
| | GAGACTACAGGTGTGTACCACC | CTCAGGTCAGAGTTCAAGAGCAGC | | |
| | ACGCCTAGCTATATTTTGCATTTTT | CCCGCCATCAAGACAAAACCTCCT | | |
| | AGTAGAGAGGAGGTTTTGTCTTG | CTCTACTAAAAATGCAAAATATAGCT | | |
| | ATGGCGGGGCTGCTCTTGAACT | AGGCGTGGTGGTACACACCTGTAG | | |
| | CTGACCTGAGGTGATCCACCCG | TCTCAGCTACGTGGGAGGCGGCA | | |
| | TCTCGACCTCCCAAAGTGCTGG | GCAGGAGAATCTCTTGAACCCGTG | | |
| | GATTACAAGCGTGTGCCACCGC | AGAAGGAGATTGCAAGGGCGAATT | | |
| | ACCAAGGGCGAATTC | C | | |
| | SEQ ID NO: 74 | SEQ ID NO: 75 | | |
| Heat | GAATTCGCCCTTGCANTCTCCTT | GAATTCGCCCTTGGGTGCGGTGGC | 219 | AluSx |
| shock_19 | CTCACGGGTTCAAGAGATTCTCC | ACACGCTTGTAATCCCAGCACTTTG | | |
| | TGCTGCCGCCTCCCACGTAGCT | GGAGGTCGAGACGGGTGGATCAC | | |
| | GAGACTACAGGTGTGTACCACC | CTCAGGTCAGAGTTCAAGAGCAGC | | |
| | ACGCCTAGCTATATTTTGCATTTTT | CCCGCCATCAAGACAAAACCTCCT | | |
| | AGTGGAGAGGAGGTTTTGTCTTG | CTCCACTAAAAATGCAAAATATAGC | | |
| | ATGGCGGGGCTGCTCTTGAACT | TAGGCGTGGTGGTACACACCTGTA | | |
| | CTGACCTGAGGTGATCCACCCG | GTCTCAGCTACGTGGGAGGCGGC | | |
| | TCTCGACCTCCCAAAGTGCTGG | AGCAGGAGAATCTCTTGAACCCGT | | |
| | GATTACAAGCGTGTGCCACCGC | GAGAAGGAGATTGCAAGGGCGAAT | | |
| | ACCAAGGGCGAATTC | TC | | |
| | SEQ ID NO: 76 | SEQ ID NO: 77 | | |
| Heat | GAATTCGCCCTTGGGTGCGGTG | ATTCGCCCTTGCAATCTCCTTCTCA | 219 | AluSx |
| shock_20 | GCACACGCTTGTAATCCCAGCA | CGGGTTCAAGAGATTCTCCTGCTG | | |
| | CTTTGGGAGGTCGAGACGGGTG | CCGCCTCCCACGTAGCTGAGACTA | | |
| | GATCACCTCAGGTCAGAGTTCAA | CAGGTGTGTACCACCACGCCTAGC | | |
| | GAGCAGCCCCGCCATCAAGAC | TATATTTTGCATTTTTAGTAGAGAGG | | |
| | AAAACCTCCTCTCTACTAAAAATG | AGGTTTTGTCTTGATGGCGGGGCTG | | |
| | CAAAATATAGCTAGGCGTGGTGG | CTCTTGAACTCTGACCTGAGGTGAT | | |
| | TACACACCTGTAGTCTCAGCTAC | CCACCCGTCTCGACCTCCCAAAGT | | |
| | GTGGGAGGCGGCAGCAGGAGA | GCTGGGATTACAAGCGTGTGCCAC | | |
| | ATCTCTTGAACCCGTGAGAAGGA | CGCACCCAAGGGCGAATTC | | |
| | GATTGCAAGGGCGAATTC | SEQ ID NO: 79 | | |
| | SEQ ID NO: 78 | | | |

TABLE 6
Oligonucleotides
Oligonucleotides used in this paper

| Oligo | | |
|---|---|---|
| # | Single guide RNA | |
| 1 | 759 sgRNA | 5′-CAAATCCTAGGCCTGCCCTG SEQ ID NO: 4 |
| 2 | 620 sgRNA | 5′-GTGAGCTGATGATCGCTCCA SEQ ID NO: 9 |
| 3 | 125.3 sgRNA | 5′-CAAAGAGAGGAAGAAGAATC SEQ ID NO: 10 |

Primers for in vitro transcription of C19MC Alu RNa

| | | 5′-Forward primer | 5′-Reverse primer |
|---|---|---|---|
| 4 | C19MC | CATCAGGACTGTGTGTTTCTGTG | TATATTTAACCATGAGAATTGAGC |
| | fragment | SEQ ID NO: 5 | SEQ ID NO: 6 |
| | chr19: | | |
| | 53,678,369- | | |
| | 53,681,590 | | |
| | (GRCh38/ | | |
| | hg38) | | |
| 5 | AluJb | TAATACGACTCACTATAGGGTGG | ACGGAGAATACTCTTGTATCACTTTGGG |
| | forward (F) | GGCGTGGTGGCTCAC | TA |
| | | SEQ ID NO: 11 | SEQ ID NO: 12 |
| 6 | AluSx | TAATACGACTCACTATAGGGCC | AGACCTCTTAGGCTAGTTGGTC |
| | forward (F) | GAGCAGGGTGGCTC | SEQ ID NO: 14 |
| | | SEQ ID NO: 13 | |
| 7 | AluSz | TAATACGACTCACTATAGGGCC | AAAGCAAGCACTTGTGTGTAGGAA |
| | forward (F) | GGGTGCGGTGGC | SEQ ID NO: 16 |
| | | SEQ ID NO: 15 | |
| 8 | AluJb | GGGCGTGGTGGCTCAC | TAATACGACTCACTATAGGGACGGAGAA |
| | reverse (R) | SEQ ID NO: 17 | TACTCTTGTATCACTTTGGGTA |
| | | | SEQ ID NO: 18 |
| 9 | AluSx | CCGAGCAGGGTGGCTC | TAATACGACTCACTATAGGGAGACCTCT |
| | reverse (R) | SEQ ID NO: 19 | TAGGCTAGTTGGTC |
| | | | SEQ ID NO: 20 |
| 10 | AluSz | CCGGGTGCGGTGGC | TAATACGACTCACTATAGGGAAAGCAAG |
| | reverse (R) | SEQ ID NO: 21 | CACTTGTGTGTAGGAA |
| | | | SEQ ID NO: 22 |

Primers for in vitro transcription of C2MC B1 RNA

| | | 5′-Forward primer | 5′-Reverse primer |
|---|---|---|---|
| 11 | C2MC B1 | GAGTCAGGAGGATCAGGAGTT | GGGGCATCACATGAATCCCA |
| | | SEQ ID NO: 23 | SEQ ID NO: 24 |
| 12 | C2MC B1 | TAATACGACTCACTATAGGGGG | GGGGCATCACATGAATCCCA |
| | forward (F) | AGTCAGGAGGATCAGGAGTT | SEQ ID NO: 8 |
| | | SEQ ID NO: 7 | |

Primers for in vitro transcription of control GFP mRNA

| | | 5′-Forward primer | 5′-Reverse primer |
|---|---|---|---|
| 13 | β- | ATTAgaattcggatccttaattaagcatgcG | ATTAgaattcGCAATGAAAATAAATGTTTTT |
| | globin3′UTR | CTCGCTTTCTTGCTGTCCAATTT | TATTAGGCAGAATCCAGAT |
| | (restriction | CTA | SEQ ID NO: 26 |
| | sites are | SEQ ID NO: 25 | |
| | shown in | | |
| | lowercase, | | |
| | and β- | | |
| | globin3′UTR | | |
| | in | | |
| | italic) | | |
| 14 | GFP IVT | CTTATGTCAATAATACGACTCAC | ATTAgaattcGCAATGAAAATAAATGTTTTT |
| | PCR | TATAGGGacatttgcttctgacacaactgt | TATTAGGCAGAATCCAGAT |
| | template | gttcactagcaacctcaaacagaccaccAT | SEQ ID NO: 28 |
| | (T7 | GGTGAGCAAGGGCGAGGAGCT | |
| | promoter | GTT | |
| | shown italic | SEQ ID NO: 27 | |
| | and 5′ UTR | | |
| | of human β- | | |
| | globin in | | |

Pimers used fo SYBR green RT-qPCR or RT-PCR

| | | 5′-Forward primer | 5′-Reverse primer |
|---|---|---|---|
| 15 | VSV | TGCAAGGAAAGCATTGAACAA | GAGGAGTCACCTGGACAATCACT |
| | | SEQ ID NO: 29 | SEQ ID NO: 30 |
| 16 | ZIKV | TGCCCAACACAAGGTGAAGC | CTCTGTCCACTAAYGTTCTTTTGC |
| | | SEQ ID NO: 31 | SEQ ID NO: 32 |
| 17 | RSV | CATCTAGCAAATACACCATCCA | TTCTGCACATCATAATTAGGAGTATCA |
| | | SEQ ID NO: 33 | SEQ ID NO: 34 |
| 18 | SARS-CoV- | GACCCCAAAATCAGCGAAAT | TCTGGTTACTGCCAGTTGAATCTG |
| | 2 | SEQ ID NO: 35 | SEQ ID NO: 36 |
| 19 | GAPDH | CTGACTTCAACAGCGACACC | TAGCCAAATTCGTTGTCATACC |
| | | SEQ ID NO: 37 | SEQ ID NO: 38 |
| 20 | Polr2a | ATCAACAATCAGCTGCGGCG | GCCAGACTTCTGCATGGCAC |
| | | SEQ ID NO: 39 | SEQ ID NO: 40 |
| 21 | Alu | CCGGGTGCGGTGGCACACGCT | GCAATCTCCTTCTCACGGGTT |
| | | SEQ ID NO: 1 | SEQ ID NO: 2 |
| 22 | IFNL2/3 | GGGGACTGCATGCCAGTGCT | CTGGCAACACAATTCAGGTCTCGC |
| | | SEQ ID NO: 41 | SEQ ID NO: 42 |

### REFERENCES

1. Ander, S. E., Diamond, M. S., and Coyne, C. B. (2019). Immune responses at the maternal-fetal interface. Sci. Immunol. 4. https://doi.org/10.1126/sciimmunol.aat6114.
2. Soares, M. J., Varberg, K. M., and Iqbal, K. (2018). Hemochorial placentation: development, function, and adaptations. Biol. Reprod. 99, 196-211.
3. Megli, C. J., and Coyne, C. B. (2022). Infections at the maternal-fetal interface: an overview of pathogenesis and defence. Nat. Rev. Microbiol. 20, 67-82.
4. Goubau, D., Deddouche, S., and Reis e Sousa, C. (2013). Cytosolic sensing of viruses. Immunity 38, 855-869.
5. Rehwinkel, J., and Gack, M. U. (2020). RIG-I-like receptors: their regulation and roles in RNA sensing. Nat. Rev. Immunol. 20, 537-551.
6. Li, D., and Wu, M. (2021). Pattern recognition receptors in health and diseases. Signal Transduct. Target. Ther. 6, 291.
7. Lazear, H. M., Schoggins, J. W., and Diamond, M. S. (2019). Shared and distinct functions of type I and type III interferons. Immunity 50, 907-923.
8. Schoggins, J. W. (2019). Interferon-stimulated genes: what do they all do? Annu. Rev. Virol. 6, 567-584.
9. Bayer, A., Lennemann, N. J., Ouyang, Y., Bramley, J. C., Morosky, S., Marques, E. T., Jr., Cherry, S., Sadovsky, Y., and Coyne, C. B. (2016). Type III interferons produced by human placental trophoblasts confer protection against zika virus infection. Cell Host Microbe 19, 705-712.
10. Corry, J., Arora, N., Good, C. A., Sadovsky, Y., and Coyne, C. B. (2017). Organotypic models of type III interferon-mediated protection from Zika virus infections at the maternal-fetal interface. Proc. Natl. Acad. Sci. USA 114, 9433-9438.
11. Jagger, B. W., Miner, J. J., Cao, B., Arora, N., Smith, A. M., Kovacs, A., Mysorekar, I. U., Coyne, C. B., and Diamond, M. S. (2017). Gestational stage and IFN-lambda signaling regulate ZIKV infection in utero. Cell Host Microbe 22, 366-376.e3.
12. Yockey, L. J., Jurado, K. A., Arora, N., Millet, A., Rakib, T., Milano, K. M., Hastings, A. K., Fikrig, E., Kong, Y., Horvath, T. L., et al. (2018). Type I interferons instigate fetal demise after Zika virus infection. Sci. Immunol. 3.
13. Yockey, L. J., and Iwasaki, A. (2018). Interferons and proinflammatory cytokines in pregnancy and fetal development. Immunity 49, 397-412.
14. Malnou, E. C., Umlauf, D., Mouysset, M., and Cavaille′, J. (2018). Imprinted microRNA gene clusters in the evolution, development, and functions of mammalian placenta. Front. Genet. 9, 706.
15. Williams, Z., Ben-Dov, I. Z., Elias, R., Mihailovic, A., Brown, M., Rosenwaks, Z., and Tuschl, T. (2013). Comprehensive profiling of circulating microRNA via small RNA sequencing of cDNA libraries reveals biomarker potential and limitations. Proc. Natl. Acad. Sci. USA 110, 4255-4260.
16. Lehnert, S., Van Loo, P., Thilakarathne, P. J., Marynen, P., Verbeke, G., and Schuit, F. C. (2009). Evidence for co-evolution between human microRNAs and Alu-repeats. PLOS One 4, e4456
17. Inoue, K., Hirose, M., Inoue, H., Hatanaka, Y., Honda, A., Hasegawa, A., Mochida, K., and Ogura, A. (2017). The rodent-specific microRNA cluster within the Sfmbt2 gene is imprinted and essential for placental development. Cell Rep. 19, 949-956.
18. Lehnert, S., Kapitonov, V., Thilakarathne, P. J., and Schuit, F. C. (2011). Modeling the asymmetric evolution of a mouse and rat-specific microRNA gene cluster intron 10 of the Sfmbt2 gene. BMC Genomics 12, 257.
19. Zhang, R., Wang, Y. Q., and Su, B. (2008). Molecular evolution of a primatespecific microRNA family. Mol. Biol. Evol. 25, 1493-1502.
20. Bortolin-Cavaille′, M. L., Dance, M., Weber, M., and Cavaille′, J. (2009). C19MC microRNAs are processed from introns of large Pol-II, non-protein-coding transcripts. Nucleic Acids Res. 37, 3464-3473.
21. Mong, E. F., Yang, Y., Akat, K. M., Canfield, J., VanWye, J., Lockhart, J., Tsibris, J. C. M., Schatz, F., Lockwood, C. J., Tuschl, T., et al. (2020). Chromosome 19 microRNA cluster enhances cell reprogramming by inhibiting epithelial-to-mesenchymal transition. Sci. Rep. 10, 3029.
22. Delorme-Axford, E., Donker, R. B., Mouillet, J. F., Chu, T., Bayer, A., Ouyang, Y., Wang, T., Stolz, D. B., Sarkar, S. N., Morelli, A. E., et al. (2013). Human placental trophoblasts confer viral resistance to recipient cells. Proc. Natl. Acad. Sci. USA 110, 12048-12053.
23. Bayer, A., Lennemann, N. J., Ouyang, Y., Sadovsky, E., Sheridan, M. A., Roberts, R. M., Coyne, C. B., and Sadovsky, Y. (2018). Chromosome 19 microRNAs exert antiviral activity independent from type III interferon signaling. Placenta 61, 33-38.
24. Chen, L. L., and Yang, L. (2017). ALUternative regulation for gene expression. Trends Cell Biol. 27, 480-490.
25. Bellemer, C., Bortolin-Cavaille′, M. L., Schmidt, U., Jensen, S. M., Kjems, J., Bertrand, E., and Cavaille′, J. (2012). Microprocessor dynamics and interactions at endogenous imprinted C19MC microRNA genes. J. Cell Sci. 125, 2709-2720.
26. Liu, W. M., Chu, W. M., Choudary, P. V., and Schmid, C. W. (1995). Cell stress and translational inhibitors transiently increase the abundance of mammalian SINE transcripts. Nucleic Acids Res. 23, 1758-1765.
27. Dhir, A., Dhir, S., Borowski, L. S., Jimenez, L., Teitell, M., Rötig, A., Crow, Y. J., Rice, G. I., Duffy, D., Tamby, C., et al. (2018). Mitochondrial doublestranded RNA triggers antiviral signalling in humans. Nature 560, 238-242.
28. Kariko′, K., Muramatsu, H., Welsh, F. A., Ludwig, J., Kato, H., Akira, S., and Weissman, D. (2008). Incorporation of pseudouridine into mRNA yields superior nonimmunogenic vector with increased translational capacity and biological stability. Mol. Ther. 16, 1833-1840.
29. Megli, C., Morosky, S., Rajasundaram, D., and Coyne, C. B. (2021). Inflammasome signaling in human placental trophoblasts regulates immune defense against Listeria monocytogenes infection. J. Exp. Med. 218.
30. Goh, X. L., Low, Y. F., Ng, C. H., Amin, Z., and Ng, Y. P. M. (2021). Incidence of SARS-COV-2 vertical transmission: a meta-analysis. Arch. Dis. Child. Fetal Neonatal Ed. 106, 112-113.
31. Wu, X., Dao Thi, V. L., Huang, Y., Billerbeck, E., Saha, D., Hoffmann, H. H., Wang, Y., Silva, L. A. V., Sarbanes, S., Sun, T., et al. (2018). Intrinsic immunity shapes viral resistance of stem cells. Cell 172. 423-438.e25 e425.
32. Lander, E. S., Linton, L. M., Birren, B., Nusbaum, C., Zody, M. C., Baldwin, J., Devon, K., Dewar, K., Doyle, M., FitzHugh, W., et al. (2001). Initial sequencing and analysis of the human genome. Nature 409, 860-921.
33. Burns, K. H. (2022). Repetitive DNA in disease. Science 376, 353-354.
34. Slotkin, R. K., and Martienssen, R. (2007). Transposable elements and the epigenetic regulation of the genome. Nat. Rev. Genet. 8, 272-285.
35. Panning, B., and Smiley, J. R. (1993). Activation of RNA polymerase III transcription of human Alu repetitive elements by adenovirus type 5: requirement for the E1b 58-kilodalton protein and the products of E4 open reading frames 3 and 6. Mol. Cell. Biol. 13, 3231-3244.
36. Panning, B., and Smiley, J. R. (1994). Activation of RNA polymerase III transcription of human Alu elements by herpes simplex virus. Virology 202, 408-417.
37. Yu, W., Hu, X., and Cao, B. (2022). Viral infections during pregnancy: the big challenge threatening maternal and fetal health. Matern. Fetal Med. 4, 72-86.
38. Bogerd, H. P., Whisnant, A. W., Kennedy, E. M., Flores, O., and Cullen, B. R. (2014). Derivation and characterization of Dicer- and microRNA-deficient human cells. RNA 20, 923-937.
39. Park, H. H., Triboulet, R., Bentler, M., Guda, S., Du, P., Xu, H., Gregory, R. I., Brendel, C., and Williams, D. A. (2018). DROSHA knockout leads to enhancement of viral titers for vectors encoding miRNA-adapted shRNAs. Mol. Ther. Nucleic Acids 12, 591-599.
40. Hwang, K. Y., and Choi, Y. B. (2016). Modulation of mitochondrial antiviral signaling by human herpesvirus 8 interferon regulatory Factor 1. J. Virol. 90, 506-520.
41. Löseke, S., Grage-Griebenow, E., Wagner, A., Gehlhar, K., and Bufe, A. (2003). Differential expression of IFN-alpha subtypes in human PBMC: evaluation of novel real-time PCR assays. J. Immunol. Methods 276, 207-222.
42. Hu, J., Lou, D., Carow, B., Winerdal, M. E., Rottenberg, M., Wikström, A. C., Norstedt, G., and Winqvist, O. (2012). LPS regulates SOCS2 transcription in a type I interferon dependent autocrine-paracrine loop. PLOS One 7, e30166.
43. Izaguirre, A., Barnes, B. J., Amrute, S., Yeow, W. S., Megjugorac, N., Dai, J., Feng, D., Chung, E., Pitha, P. M., and Fitzgerald-Bocarsly, P. (2003). Comparative analysis of IRF and IFN-alpha expression in human plasmacytoid and monocyte-derived dendritic cells. J. Leukoc. Biol. 74, 1125-1138.
44. Konermann, S., Brigham, M. D., Trevino, A. E., Joung, J., Abudayyeh, O. O., Barcena, C., Hsu, P. D., Habib, N., Gootenberg, J. S., Nishimasu, H., et al. (2015). Genome-scale transcriptional activation by an engineered CRISPR-Cas9 complex. Nature 517, 583-588.
45. Daniloski, Z., Jordan, T. X., Wessels, H. H., Hoagland, D. A., Kasela, S., Legut, M., Maniatis, S., Mimitou, E. P., Lu, L., Geller, E., et al. (2021). Identification of required Host Factors for SARS-COV-2 Infection in Human Cells. Cell 184, 92-105.e16.
46. Rubinson, D. A., Dillon, C. P., Kwiatkowski, A. V., Sievers, C., Yang, L., Kopinja, J., Rooney, D. L., Zhang, M., Ihrig, M. M., McManus, M. T., et al. (2003). A lentivirus-based system to functionally silence genes in primary mammalian cells, stem cells and transgenic mice by RNA interference. Nat. Genet. 33, 401-406.
47. Love, M. I., Huber, W., and Anders, S. (2014). Moderated estimation of fold change and dispersion for RNA-seq data with DESeq2. Genome Biol. 15, 550.
48. Widmann, J., Stombaugh, J., McDonald, D., Chocholousova, J., Gardner, P., Iyer, M. K., Liu, Z., Lozupone, C. A., Quinn, J., Smit, S., et al. (2012). RNASTAR: an RNA STructural Alignment Repository that provides insight into the evolution of natural and artificial RNAs. RNA 18, 1319-1327.
49. Liao, Y., Smyth, G. K., and Shi, W. (2014). featureCounts: an efficient general purpose program for assigning sequence reads to genomic features. Bioinformatics 30, 923-930.
50. Robinson, M. D., McCarthy, D. J., and Smyth, G. K. (2010). edgeR: a Bioconductor package for differential expression analysis of digital gene expression data. Bioinformatics 26, 139-140.
51. Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc. Natl. Acad. Sci. USA 102, 15545-15550.
52. Schneider, C. A., Rasband, W. S., and Eliceiri, K. W. (2012). NIH Image to ImageJ: 25 years of image analysis. Nat. Methods 9, 671-675.
53. Ohinata, Y., and Tsukiyama, T. (2014). Establishment of trophoblast stem cells under defined culture conditions in mice. PLOS One 9, e107308.
54. Lockhart, J., Canfield, J., Mong, E. F., VanWye, J., and Totary-Jain, H. (2019). Nucleotide modification alters microRNA-dependent silencing of microRNA switches. Mol. Ther. Nucleic Acids 14, 339-350.
55. Lockhart, J. H., VanWye, J., Banerjee, R., Wickline, S. A., Pan, H., and Totary-Jain, H. (2021). Self-assembled miRNA-switch nanoparticles target denuded regions and prevent restenosis. Mol. Ther. 29, 1744-1757.
56. Canfield, J., Arlier, S., Mong, E. F., Lockhart, J., VanWye, J., Guzeloglu-Kayisli, O., Schatz, F., Magness, R. R., Lockwood, C. J., Tsibris, J. C. M., et al. (2019). Decreased LIN28B in preeclampsia impairs human trophoblast differentiation and migration. FASEB J. 33, 2759-2769.
57. Livak, K. J., and Schmittgen, T. D. (2001). Analysis of relative gene expression data using real-time quantitative PCR and the 2 (−Delta Delta C(T)) method. Methods 25, 402-408.
58. Ozmen, A., Guzeloglu-Kayisli, O., Tabak, S., Guo, X., Semerci, N., Nwabuobi, C., Larsen, K., Wells, A., Uyar, A., Arlier, S., et al. (2022). Preeclampsia is associated with reduced ISG15 levels impairing extravillous trophoblast invasion. Front. Cell Dev. Biol. 10, 898088.
59. Mong, E. F., Akat, K. M., Canfield, J., Lockhart, J., VanWye, J., Matar, A., Tsibris, J. C. M., Wu, J. K., Tuschl, T., and Totary-Jain, H. (2018). Modulation of LIN28B/Let-7 signaling by propranolol contributes to infantile hemangioma involution. Arterioscler. Thromb. Vasc. Biol. 38, 1321-1332.
60. Means, R. E., Roy, S. G., and Katz, S. G. (2020). The propagation and quantification of two emerging oncolytic viruses: vesicular stomatitis (VSV) and Zika (ZIKV). Methods Mol. Biol. 2097, 253-263. 61. Agbulos, D. S., Barelli, L., Giordano, B. V., and Hunter, F. F. (2016). Zika virus: quantification, propagation, detection, and storage. Curr. Protoc. Microbiol. 43, 15D.4.1-15D.4.16. https://doi.org/10.1002/cpmc.19.
62. Jenkins, T., Wang, R., Harder, O., Xue, M., Chen, P., Corry, J., Walker, C., Teng, M., Mejias, A., Ramilo, O., et al. (2021). A novel live attenuated respiratory syncytial virus vaccine candidate with mutations in the L protein SAM Binding Site and the G protein cleavage site is protective in cotton rats and a rhesus macaque. J. Virol. 95.
63. Case, J. B., Bailey, A. L., Kim, A. S., Chen, R. E., and Diamond, M. S. (2020). Growth, detection, quantification, and inactivation of SARS-COV-2. Virology 548, 39-48.
64. McKimm-Breschkin, J. L. (2004). A simplified plaque assay for respiratory syncytial virus-direct visualization of plaques without immunostaining. J. Virol. Methods 120, 113-117.
65. Mendoza, E. J., Manguiat, K., Wood, H., and Drebot, M. (2020). Two detailed plaque assay protocols for the quantification of infectious SARS-COV-2. Curr. Protoc. Microbiol. 57, ecpmc105.
66. Farazi, T. A., Brown, M., Morozov, P., Ten Hoeve, J. J., Ben-Dov, I. Z., Hovestadt, V., Hafner, M., Renwick, N., Mihailovi_c, A., Wessels, L. F., et al. (2012). Bioinformatic analysis of barcoded cDNA libraries for small RNA profiling by next-generation sequencing. Methods 58, 171-187.
67. Max, K. E. A., Wang, V. R., Chang, M. S., Liau, J., Weiss, Z. R., Morgan, S., Li, J., Bogardus, K. A., Morozov, P., Suryawanshi, H., et al. (2021). Plasma microRNA interindividual variability in healthy individuals, pregnant women, and an individual with a stably altered neuroendocrine phenotype.
68. U.S. Pat. Nos. 11,357,789 and 11,844,809 to Hana Totary-Jain

1. A method for reducing the likelihood of amniotic fluid infection and/or fetal infection, or treating amniotic fluid infection and/or fetal infection, the method comprising: vaginally administering to a pregnant female a composition comprising an in vitro transcribed (IVT) RNA comprising short interspersed nuclear elements (SINEs), or fragments thereof, wherein the SINEs or fragments thereof comprise Alu repeats.

2. The method of claim 1, wherein the administration is topical.

3. (canceled)

4. The method of claim 1, wherein the infection is diagnosed as viral, bacterial, or fungal.

5-6. (canceled)

7. A method for preparing a cell, tissue, or an organ for transplant in a recipient, the method comprising: contacting the cell, tissue, or organ with a composition comprising an in vitro transcribed (IVT) RNA comprising short interspersed nuclear elements (SINEs), or fragments thereof, wherein the SINEs or fragments thereof comprise Alu repeats.

8. The method of claim 7, wherein the recipient is administered IVT RNA comprising SINEs, or fragments thereof, prior to transplantation.

9. The method of claim 8, wherein administration of the IVT RNA comprising SINEs, or fragments thereof, enhances immunotolerance in the recipient.

10. A method of treating wounded tissue in a subject in need thereof, the method comprising: administering to the wounded tissue a composition comprising an in vitro transcribed (IVT) RNA comprising short interspersed nuclear elements (SINEs), or fragments thereof, wherein the SINEs or fragments thereof comprise Alu repeats.

11. The method of claim 10, wherein
(a) the wound is a skin wound, and the composition is administered topically; or
(b) the wound is in lung tissue, and the composition is administered via inhalation; or
(c) the wound is an eye wound, and the composition is administered topically to the wounded eye.

12-13. (canceled)

14. The method of claim 1, wherein the IVT RNA comprises at least one modified nucleotide, wherein the at least one modified nucleotide is selected from the group consisting of pseudouridine, Nl-methylpseudouridine, 5-methylcytosine (m5C), S-methyluridine (m5U), 2′-O-methyluridine (Um or m2′OU), 2-thiouridine (s2U), or N6-methyladenosine (m6A), and combinations thereof.

15-19. (canceled)

20. The method of claim 1 comprising, prior to administration, determining the level of Alu RNA in a biological sample from the subject.

21. The method of claim 20, wherein detecting the level of Alu RNA comprises measuring the level of a small cytoplasmic Alu (sc-Alu) RNA in the sample and/or measuring the level of full length Alu (fl-Alu) RNA in the sample.

22. The method of claim 21, further comprising comparing the amount of sc-Alu RNA to fl-Alu RNA.

23. (canceled)

24. The method of claim 20, wherein the biological sample is from a tissue or organ biopsy, placenta, blood, serum, plasma, vaginal discharge, urine, lymphatic fluids or amniotic fluid.

25. The method of claim 7, wherein the IVT RNA comprises at least one modified nucleotide, wherein the at least one modified nucleotide is selected from the group consisting of pseudouridine, NI-methylpseudouridine, 5-methylcytosine (m5C), 5-methyluridine (m5U), 2′-O-methyluridine (Um or m2′OU), 2-thiouridine (s2U), or N6-methyladenosine (m6A), and combinations thereof.

26. The method of claim 7 comprising, prior to administration, determining the level of Alu RNA in a biological sample from the subject.

27. The method of claim 7, wherein detecting the level of Alu RNA comprises measuring the level of a small cytoplasmic Alu (sc-Alu) RNA in the sample and/or measuring the level of full length Alu (fl-Alu) RNA in the sample.

28. The method of claim 9, wherein the recipient is immunocompromised.

29. The method of claim 28 wherein the recipient has immunodeficiency disorder.


SEQUENCE LISTING (82 sequences)

SEQ ID NO: 1 - DNA, 21 bp, synthetic construct
  source 1..21: mol_type = other DNA
ccgggtgcggtggcacacgct

SEQ ID NO: 2 - DNA, 21 bp, synthetic construct
  source 1..21: mol_type = other DNA
gcaatctccttctcacgggtt

SEQ ID NO: 3 - DNA, 17 bp, synthetic construct
  source 1..17: mol_type = other DNA
cactgcactccagcctg

SEQ ID NO: 4 - DNA, 20 bp, synthetic construct
  source 1..20: mol_type = other DNA
caaatcctaggcctgccctg

SEQ ID NO: 5 - DNA, 23 bp, synthetic construct
  source 1..23: mol_type = other DNA
catcaggactgtgtgtttctgtg

SEQ ID NO: 6 - DNA, 24 bp, synthetic construct
  source 1..24: mol_type = other DNA
tatatttaaccatgagaattgagc

SEQ ID NO: 7 - DNA, 42 bp, synthetic construct
  source 1..42: mol_type = other DNA
taatacgactcactatagggggagtcaggaggatcaggagtt

SEQ ID NO: 8 - DNA, 20 bp, synthetic construct
  source 1..20: mol_type = other DNA
ggggcatcacatgaatccca

SEQ ID NO: 9 - DNA, 20 bp, synthetic construct
  source 1..20: mol_type = other DNA
gtgagctgatgatcgctcca

SEQ ID NO: 10 - DNA, 20 bp, synthetic construct
  source 1..20: mol_type = other DNA
caaagagaggaagaagaatc

SEQ ID NO: 11 - DNA, 38 bp, synthetic construct
  source 1..38: mol_type = other DNA
taatacgactcactatagggtggggcgtggtggctcac

SEQ ID NO: 12 - DNA, 30 bp, synthetic construct
  source 1..30: mol_type = other DNA
acggagaatactcttgtatcactttgggta

SEQ ID NO: 13 - DNA, 36 bp, synthetic construct
  source 1..36: mol_type = other DNA
taatacgactcactatagggccgagcagggtggctc

SEQ ID NO: 14 - DNA, 22 bp, synthetic construct
  source 1..22: mol_type = other DNA
agacctcttaggctagttggtc

SEQ ID NO: 15 - DNA, 34 bp, synthetic construct
  source 1..34: mol_type = other DNA
taatacgactcactatagggccgggtgcggtggc

SEQ ID NO: 16 - DNA, 24 bp, synthetic construct
  source 1..24: mol_type = other DNA
aaagcaagcacttgtgtgtaggaa

SEQ ID NO: 17 - DNA, 16 bp, synthetic construct
  source 1..16: mol_type = other DNA
gggcgtggtggctcac

SEQ ID NO: 18 - DNA, 50 bp, synthetic construct
  source 1..50: mol_type = other DNA
taatacgactcactatagggacggagaatactcttgtatcactttgggta

SEQ ID NO: 19 - DNA, 16 bp, synthetic construct
  source 1..16: mol_type = other DNA
ccgagcagggtggctc

SEQ ID NO: 20 - DNA, 42 bp, synthetic construct
  source 1..42: mol_type = other DNA
taatacgactcactatagggagacctcttaggctagttggtc

SEQ ID NO: 21 - DNA, 14 bp, synthetic construct
  source 1..14: mol_type = other DNA
ccgggtgcggtggc

SEQ ID NO: 22 - DNA, 44 bp, synthetic construct
  source 1..44: mol_type = other DNA
taatacgactcactatagggaaagcaagcacttgtgtgtaggaa

SEQ ID NO: 23 - DNA, 21 bp, synthetic construct
  source 1..21: mol_type = other DNA
gagtcaggaggatcaggagtt

SEQ ID NO: 24 - DNA, 20 bp, synthetic construct
  source 1..20: mol_type = other DNA
ggggcatcacatgaatccca

SEQ ID NO: 25 - DNA, 57 bp, synthetic construct
  source 1..57: mol_type = other DNA
attagaattcggatccttaattaagcatgcgctcgctttcttgctgtccaatttcta

SEQ ID NO: 26 - DNA, 50 bp, synthetic construct
  source 1..50: mol_type = other DNA
attagaattcgcaatgaaaataaatgttttttattaggcagaatccagat

SEQ ID NO: 27 - DNA, 107 bp, synthetic construct
  source 1..107: mol_type = other DNA
cttatgtcaataatacgactcactatagggacatttgcttctgacacaactgtgttcactagcaacctcaaacagaccaccatggtgagcaagggcgaggagctgtt

SEQ ID NO: 28 - DNA, 50 bp, synthetic construct; identical to SEQ ID NO: 26
  source 1..50: mol_type = other DNA

SEQ ID NO: 29 - DNA, 21 bp, synthetic construct
  source 1..21: mol_type = other DNA
tgcaaggaaagcattgaacaa

SEQ ID NO: 30 - DNA, 23 bp, synthetic construct
  source 1..23: mol_type = other DNA
gaggagtcacctggacaatcact

SEQ ID NO: 31 - DNA, 20 bp, synthetic construct
  source 1..20: mol_type = other DNA
tgcccaacacaaggtgaagc

SEQ ID NO: 32 - DNA, 24 bp, synthetic construct
  source 1..24: mol_type = other DNA
ctctgtccactaaygttcttttgc

SEQ ID NO: 33 - DNA, 22 bp, synthetic construct
  source 1..22: mol_type = other DNA
catctagcaaatacaccatcca

SEQ ID NO: 34 - DNA, 27 bp, synthetic construct
  source 1..27: mol_type = other DNA
ttctgcacatcataattaggagtatca

SEQ ID NO: 35 - DNA, 20 bp, synthetic construct
  source 1..20: mol_type = other DNA
gaccccaaaatcagcgaaat

SEQ ID NO: 36 - DNA, 24 bp, synthetic construct
  source 1..24: mol_type = other DNA
tctggttactgccagttgaatctg

SEQ ID NO: 37 - DNA, 20 bp, synthetic construct
  source 1..20: mol_type = other DNA
ctgacttcaacagcgacacc

SEQ ID NO: 38 - DNA, 22 bp, synthetic construct
  source 1..22: mol_type = other DNA
tagccaaattcgttgtcatacc

SEQ ID NO: 39 - DNA, 20 bp, synthetic construct
  source 1..20: mol_type = other DNA
atcaacaatcagctgcggcg

SEQ ID NO: 40 - DNA, 20 bp, synthetic construct
  source 1..20: mol_type = other DNA
gccagacttctgcatggcac

SEQ ID NO: 41 - DNA, 20 bp, synthetic construct
  source 1..20: mol_type = other DNA
ggggactgcatgccagtgct

SEQ ID NO: 42 - DNA, 24 bp, synthetic construct
  source 1..24: mol_type = other DNA
ctggcaacacaattcaggtctcgc

SEQ ID NO: 43 - DNA, 240 bp, synthetic construct
  source 1..240: mol_type = other DNA
gaattcgcccttgtgcggtggcacacgcttgtaatcccagcactttgggaggtcgagacgggtggatcacctcaggtcagagttcaagagcagccccgccatcaagacaaaacctcctctctactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 44 - DNA, 244 bp, synthetic construct
  source 1..244: mol_type = other DNA
gaattcgcccttgcantctccttctcacgggttcaagagattctcctgctgccgcctcccacgtagctgagactacaggtgtgtactaccacgcctagctatattttgcatttttagtagagaggaggttttgtcttgatggcggggctgctcttgaactctgacctgaggtgatccacccgtctcgacctcccaaagtgctgggattacaagcgtgtgccaccgcacccggaagggcgaattc

SEQ ID NO: 45 - DNA, 244 bp, synthetic construct
  source 1..244: mol_type = other DNA
gaattcgcccttccgggtgcggtggcacacgcttgtaatcccagcactttgggaggtcgagacgggtggatcacctcaggtcagagttcaagagcagccccgccatcaagacaaaacctcctctctactaaaaatgcaaaatatagctaggcgtggtagtacacacctgtagtctcagctacgtgggaggcggcagcaggagaatctcttgaacccgtgagaaggagattgcaagggcgaattc

SEQ ID NO: 46 - DNA, 242 bp, synthetic construct
  source 1..242: mol_type = other DNA
gaattcgcccttgcatnctccttctcacgggttcaagagattctcctgctgccgcctcccacgtagtctgagactacaggtgtgtaccaccacgcctagctatattttgcatttttagtagagaggaggttttatcttgatggcggggctgctcttgaactctgacctgaggtgatccacccgtctcgacctcccaaagtgctgggattacaagcgtgtgccaccgcaccaagggcgaattc

SEQ ID NO: 47 - DNA, 241 bp, synthetic construct
  source 1..241: mol_type = other DNA
gaattcgcccttggtgcggtggcacacgcttgtaatcccagcactttgggaggtcgagacgggtggatcacctcaggtcagagttcaagagcagccccgccatcaagataaaacctcctctctactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 48 - DNA, 259 bp, synthetic construct
  source 1..259: mol_type = other DNA
gattcccttgcaatnctccttctcacgggttcaagagattctcctgctgccgcctcccacgcagctgagactacaggtgtgtaccaccacgcctagctatattttgcatttttagtagagaggaggttttgtcttgatggcggggctgctcttgaactctgacctgaggtgatccgcccgcctcgacctcccaagctgctgttattacgatcccgtgccaccgccatagtgaggacgactccgcctcaaccttcaattc

SEQ ID NO: 49 - DNA, 244 bp, synthetic construct
  source 1..244: mol_type = other DNA
gaattcgcccttccgggtgcggtggcacacgcttgtaatcccagcactttgggaggtcgagacgggcggatcacctcaggtcagagttcaagagcagccccgccatcaagacaaaacctcctctctactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 50 - DNA, 241 bp, synthetic construct
  source 1..241: mol_type = other DNA
gaattcgcccttgggtgcggtggcacacgcttgtaatcccagcactttgggaggccaaggcgggcagatcatgaggtcaggaaattgagaccagcctggctaacatggtgaaactctgtctctactaaaaatacagaaaattagctgggcatggtggccagcgcctgtagtcccaggtacttggaggctgaggcaggagaattgcttgaacccgtgagaaggagattgcaagggcgaattc

SEQ ID NO: 51 - DNA, 110 bp, synthetic construct
  source 1..110: mol_type = other DNA
gaattcgcccttgcantctccttctcacgggttcaagcgattctcctgcctcagcctcctgaatagctgggaccacaagcgtgtgccaccgcacccggaagggcgaattc

SEQ ID NO: 52 - DNA, 110 bp, synthetic construct; identical to SEQ ID NO: 51
  source 1..110: mol_type = other DNA

SEQ ID NO: 53 - DNA, 243 bp, synthetic construct
  source 1..243: mol_type = other DNA
gaattcgcccttgcantctccttctcacgggttcaagcgattctcctgcctcagcctcccaactaactgggactacaagcgcgcgccaccacgcccagctaatttttgtatttttagtagagacggggcttcaccatgttggccaggatggtctcgatctcctgacctcgtgatccgcccgcctcgacctcccaaagtgctgggattacaagcgtgtgccaccgcacccggaagggcgaattc

SEQ ID NO: 54 - DNA, 244 bp, synthetic construct
  source 1..244: mol_type = other DNA
gaattcgcccttccgggtgcggtggcacacgcttgtaatcccagcactttgggaggtcgaggccgggcggatcacgaggtcaggagatcgagaccatcctggccaacatggtgaagccccgtctctactaaaaatacaaaaattagctgggcgtggtggcgcgcgcttgtagtcccagttagttgggaggctgaggcaggagaatcgcttgaacccgtgagaaggagattgcaagggcgaattc

SEQ ID NO: 55 - DNA, 244 bp, synthetic construct
  source 1..244: mol_type = other DNA
gaattcgcccttgcantctccttctcacgggttcaagagatcctcctgctgccgcctcccacgtagctgagactacaggtgtgtaccaccacgcctagctatattttgcatttttagtagagaggaggttttgtcttgatggcggggctgctcttggactctgacctgaggtgatccacccgtctcgacctcccaaagtgctgggattacaagcgtgtgccaccgcacccggaagggcgaattc

SEQ ID NO: 56 - DNA, 244 bp, synthetic construct
  source 1..244: mol_type = other DNA
gaattcgcccttccgggtgcggtggcacacgcttgtaatcccagcactttgggaggtcgagacgggtggatcacctcaggtcagagtccaagagcagccccgccatcaagacaaaacctcctctctactaaaaatgcaaaatatagctaggcgtggtggtacacacctgtagtctcagctacgtgggaggcggcagcaggaggatctcttgaacccgtgagaaggagattgcaagggcgaattc

SEQ ID NO: 57 - DNA, 241 bp, synthetic construct
  source 1..241: mol_type = other DNA
gaattcgcccttcgtgcggtggcacacgcttgtaatcccagcactttgggaggtcgagacgggtggatcgcctcaggtcagagttcaagagcagccccgccatcaagacaaaacctcctctctactaaaaatgcaaaatatagctaggcgtggtggttcacacctgtagtctcagctacgtgggaggcggcagcaggagaatctcttgaacccgtgagaaggagattgcaagggcgaattc

SEQ ID NO: 58 - DNA, 242 bp, synthetic construct
  source 1..242: mol_type = other DNA
gaattcgcccttgcantctccttctcacgggttcaagagattctcctgctgccgcctcccacgtagtctgagactacaggtgtgaaccaccacgcctagctatattttgcatttttagtagagaggaggttttgtcttgatggcggggctgctcttgaactctgacctgaggcgatccacccgtctcgacctcccaaagtgctgggattacaagcgtgtgccaccgcacgaagggcgaattc

SEQ ID NO: 59 - DNA, 244 bp, synthetic construct
  source 1..244: mol_type = other DNA
gaattcgcccttgggtgcggtggcacacgcttgtaataccagcactttgggaggccaaggtgggcggatcacctgaggtcaggagttcaagaccagcctggccaatatggtgaaagtccgtctctactaaaattacaaaaattagccgggcgtggtggcagatcccttgtagtcccagctactcgggaggctgaggcaggagaatagcttgaacccgtgagaaggagattgcaagggcgaattc

SEQ ID NO: 60 - DNA, 242 bp, synthetic construct
  source 1..242: mol_type = other DNA
aattcgcccttgcantctccttctcacgggttcaagctattctcctgcctcagcctcccgagtagctgggactacaagggatctgccaccacgcccggctaatttttgtaattttagtagagacggactttcaccatattggccaggctggtcttgaactcctgacctcaggtgatccgcccaccttggcctcccaaagtgctggtattacaagcgtgtgccaccgcaccaagggcgaattc

SEQ ID NO: 61 - DNA, 240 bp, synthetic construct
  source 1..240: mol_type = other DNA
gaattcgcccttgcaatctccttctcacgggttcaagagactctcctgctgccgcctcccacgtagctgagactacaggtgtgtaccaccacgcctagctatattttgcatttttagtagagaggaggttttgtcttgatggcggggctgctcttgaactctgacctgaggtgatccacccgtctcgacctcccaaagtgctgggattacaagcgtgtgccaccgcacccggaagggcga

SEQ ID NO: 62 - DNA, 244 bp, synthetic construct
  source 1..244: mol_type = other DNA
gaattcgcccttccgggtgcggtggcacacgcttgtaatcccagcactttgggaggtcgagacgggtggatcacctcaggtcagagttcaagagcagccccgccatcaagacaaaacctcctctctactaaaaatgcaaaatatagctaggcgtggtggtacacacctgtagtctcagctacgtgggaggcggcagcaggagagtctcttgaacccgtgagaaggagattgcaagggcgaattc

SEQ ID NO: 63 - DNA, 244 bp, synthetic construct
  source 1..244: mol_type = other DNA
gaattcgcccttccgggtgcggtggcacacgcttgtaatcccagcactttgggaggtcgagacgggtggatcacctcaggtcagagttcaagagcagccccgccatcaagacaaaacctcctctctactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 64 - DNA, 242 bp, synthetic construct
  source 1..242: mol_type = other DNA
attcgcccttgcantctccttctcacgggttcaagagattctcctgctgccgcctcccacgtagctgagactacaggtgtgtaccaccacgcctagctatattttgcatttttagtagagaggaggttttgtcttgatggcggggctgctcttgaactctgacctgaggtgatccacccgtctcgacctcccaaagtgctgggattacaagcgtgtgccaccgcacccggaagggcgaattc

SEQ ID NO: 65 - DNA, 110 bp, synthetic construct
  source 1..110: mol_type = other DNA
gaattcgcccttgcaatctccttctcacgggttcaagcgattctcctgcctcagcctcctgagtagctgggattacaagcgtgtgccaccgcacccggaagggcgaattc

SEQ ID NO: 66 - DNA, 244 bp, synthetic construct
  source 1..244: mol_type = other DNA
gaattcgcccttgcaatctccttctcacgggttcaagagattctcctgctgccgcctcccacgtagctgagactacaggtgtgtaccaccacgcctagctatattttgcatttttagtagagaggaggttttgtcttgatggcggggctgctctcgaactctgacctgaggtgatccacccgtctcgacctcccaaagtgctgggattacaagcgtgtgccaccgcacccggaagggcgaattc

SEQ ID NO: 67 - DNA, 244 bp, synthetic construct
  source 1..244: mol_type = other DNA
gaattcgcccttccgggtgcggtggcacacgcttgtaatcccagcactttgggaggtcgagacgggtggatcacctcaggtcagagttcgagagcagccccgccatcaagacaaaacctcctctctactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 68 - DNA, 244 bp, synthetic construct
  source 1..244: mol_type = other DNA
gaattcgcccttgcaatctccttctcacgggttcaagagattctcctgctgccgcctcccacgtagctgagactacaggtgtgtaccaccacgcctagctatattttgcatttttagtagagaggaggttttgtcttgatggcggggctgctcttgaactctgacctgaggtgatccacccgtctcggcctcccaaagtgctgggattacaagcgtgtgccaccgcacccggaagggcgaattc

SEQ ID NO: 69 - DNA, 242 bp, synthetic construct
  source 1..242: mol_type = other DNA
attcgcccttccgggtgcggtggcacacgcttgtaatcccagcactttgggaggccgagacgggtggatcacctcaggtcagagttcaagagcagccccgccatcaagacaaaacctcctctctactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 70 - DNA, 239 bp, synthetic construct
  source 1..239: mol_type = other DNA
gaattcgcccttgggtgcggtggcacacgcttgtaatcccagcactttgggaggccgaggtgagcggatcatgaggtcaggagtttgagaccagcctggccaacacagtgaaaacccgtctactaaaagtacaaaaattagctgggcatggtggcaggcgcctgtaataccagctactcgggagactgaggcaggagaatcgcttgaacccgtgagaaggagattgcaagggcgaattc

SEQ ID NO: 71 - DNA, 236 bp, synthetic construct
  source 1..236: mol_type = other DNA
attcgcccttgcaatctccttctcacgggttcaagcgattctcctgcctcagtctcccgagtagctggtattacaggcgcctgccaccatgcccagctaatttttgtacttttagtagacgggttttcactgtgttggccaggctggtctcaaactcctgacctcatgatccgctcacctcggcctcccaaagtgctgggattacaagcgtgtgccaccgcaccaagggcgaattc

SEQ ID NO: 72 - DNA, 241 bp, synthetic construct
  source 1..241: mol_type = other DNA
gaattcgcccttgcantctccttctcacgggttcaagcgattctcctgcctcagcctcccaactaactgggactacaagcgcgcgccaccacgcccagctaatttttgtatttttagtagagacggggcttcaccatgttggccaggatggtctcgatctcctgacctcgtgatccgcccgcctcgacctcccaaagtgctgggattacaagcgtgtgccaccgcacccaagggcgaattc

SEQ ID NO: 73 - DNA, 239 bp, synthetic construct
  source 1..239: mol_type = other DNA
attcgcccttgggtgcggtggcacacgcttgtaatcccagcactttgggaggtcgaggcgggcggatcacgaggtcaggagatcgagaccatcctggccaacatggtgaagccccgtctctactaaaaatacaaaaattagctgggcgtggtggcgcgcgcttgtagtcccagttagttgggaggctgaggcaggagaatcgcttgaacccgtgagaaggagattgcaagggcgaattc

SEQ ID NO: 74 - DNA, 241 bp, synthetic construct
  source 1..241: mol_type = other DNA
gaattcgcccttgcaatctccttctcacgggttcaagagattctcctgctgccgcctcccacgtagctgagactacaggtgtgtaccaccacgcctagctatattttgcatttttagtagagaggaggttttgtcttgatggcggggctgctcttgaactctgacctgaggtgatccacccgtctcgacctcccaaagtgctgggattacaagcgtgtgccaccgcaccaagggcgaattc

SEQ ID NO: 75 - DNA, 242 bp, synthetic construct
  source 1..242: mol_type = other DNA
gaattcgcccttgggtgcggtggcacacgcttgtaatcccagcactttgggaggtcgagacgggtggatcacctcaggtcagagttcaagagcagccccgccatcaagacaaaacctcctctctactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 76 - DNA, 241 bp, synthetic construct
  source 1..241: mol_type = other DNA
gaattcgcccttgcantctccttctcacgggttcaagagattctcctgctgccgcctcccacgtagctgagactacaggtgtgtaccaccacgcctagctatattttgcatttttagtggagaggaggttttgtcttgatggcggggctgctcttgaactctgacctgaggtgatccacccgtctcgacctcccaaagtgctgggattacaagcgtgtgccaccgcaccaagggcgaattc

SEQ ID NO: 77 - DNA, 242 bp, synthetic construct
  source 1..242: mol_type = other DNA
gaattcgcccttgggtgcggtggcacacgcttgtaatcccagcactttgggaggtcgagacgggtggatcacctcaggtcagagttcaagagcagccccgccatcaagacaaaacctcctctccactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 78 - DNA, 242 bp, synthetic construct; identical to SEQ ID NO: 75
  source 1..242: mol_type = other DNA

SEQ ID NO: 79 - DNA, 240 bp, synthetic construct
  source 1..240: mol_type = other DNA
attcgcccttgcaatctccttctcacgggttcaagagattctcctgctgccgcctcccacgtagctgagactacaggtgtgtaccaccacgcctagctatattttgcatttttagtagagaggaggttttgtcttgatggcggggctgctcttgaactctgacctgaggtgatccacccgtctcgacctcccaaagtgctgggattacaagcgtgtgccaccgcacccaagggcgaattc

SEQ ID NO: 80 - DNA, 25 bp, synthetic construct
  source 1..25: mol_type = other DNA
cttgaaggacagacatgactttgga

SEQ ID NO: 81 - DNA, 21 bp, synthetic construct
  source 1..21: mol_type = other DNA
ggatggtttcagccttttgga

SEQ ID NO: 82 - DNA, 24 bp, synthetic construct
  source 1..24: mol_type = other DNA
ttccccaggaggagtttggcaacc